(19) United States
(12) Patent Application Publication
(10) Pub. No.: US 2022/0281930 A1
(43) Pub. Date: Sep. 8, 2022

(54) COMPOSITIONS AND METHODS FOR THE TREATMENT OF PATHOLOGICAL PAIN AND ITCH

(71) Applicant: DUKE UNIVERSITY, Durham, NC (US)

(72) Inventor: Wolfgang Liedtke, Durham, NC (US)

(73) Assignee: DUKE UNIVERSITY, Durham, NC (US)

(21) Appl. No.: 17/637,760

(22) PCT Filed: Aug. 24, 2020
(86) PCT No.: PCT/US2020/047655

§ 371 Date: Feb. 23, 2022

Related U.S. Application Data
(60) Provisional application No. 62/890,710, filed on Aug. 23, 2019.

(51) Int. Cl. C07K 14/47; C07K 14/465; C07K 14/005; A61K 48/00; A61K 31/55; A61K 31/335; A61K 31/167; A61K 31/426; A61K 33/24; A61K 31/506; A61K 31/404; A61K 31/517; A61K 31/497; A61K 31/5377; A61K 31/433; A61K 31/5517; A61K 45/06; A61K 9/00; A61P 25/04; C12N 7/00; C12N 15/86

(52) U.S. Cl. CPC C07K 14/4702; C07K 14/465; C07K 14/005; A61K 48/0058; A61K 31/55; A61K 31/335; A61K 31/167; A61K 31/426; A61K 33/24; A61K 31/506; A61K 31/404; A61K 31/517; A61K 31/497; A61K 31/5377; A61K 31/433; A61K 31/5517; A61K 45/06; A61K 9/0019; A61P 25/04; C12N 7/00; C12N 15/86; A61K 38/00

(57) ABSTRACT

The present disclosure provides, in part, composition and methods for the treatment of pathological pain and itch.

### CROSS REFERENCE TO RELATED APPLICATIONS

This application claims priority to U.S. Provisional Patent Application Ser. No. 62/890,710, filed Aug. 23, 2019, the contents of which is hereby incorporated by reference in its entirety.

### FEDERAL FUNDING LEGEND

This invention was made with government support under Grant Nos. NS066307, DE11794, and DE022793, all from the National Institutes of Health (NIH). The Federal Government has certain rights to this invention.

### INCORPORATION BY REFERENCE OF SEQUENCE LISTING PROVIDED ELECTRONICALLY

This application contains a Sequence Listing submitted as an electronic text file named “05_20-1246-WO_Sequence-Listing_ST25.txt”, having a size in bytes of 114 kb, and created on Aug. 24, 2020. The information contained in this electronic file is hereby incorporated by reference in its entirety.

### BACKGROUND

### Field

The present disclosure provides compositions and methods for the treatment of pathological pain and itch.

### Description of the Related Art

Inhibitory GABA-ergic neurotransmission is of fundamental relevance for the adult vertebrate central nervous system and requires low chloride ion concentration in neurons. In the mature vertebrate central nervous system (CNS), γ-aminobutyric acid (GABA) acts primarily as an inhibitory neurotransmitter that is critical for normal CNS functioning. In chronic pain and itch, GABA-ergic transmission is compromised, causing circuit malfunction and disrupting inhibitory networks. Therefore, the need arises to discover new approaches to restore physiologic GABA-ergic transmission. This would increase the basic understanding of these sensory disorders to address the unmet medical need of chronic pain and itch, with safer and more effective alternatives to opioids for chronic pain.

In the adult vertebrate CNS, the K⁺/Cl⁻ cotransporter (KCC2) is expressed in neurons, continuously extruding chloride ions, thus ensuring that intracellular levels of chloride ions remain low, which is essential for inhibitory GABA-ergic neurotransmission. In chronic pathologic pain and other neuro-psychiatric illnesses, KCC2 expression is attenuated in the primary sensory gate in spinal cord dorsal horn neurons. This process is one key pathophysiological mechanism that contributes to an excitation/inhibition imbalance, more specifically it corrupts inhibitory neurotransmission and causes inhibitory circuit malfunction. Notably, there is no “back-up” protein that can rescue the KCC2 expression deficit.

Thus, there is a need for novel therapies and methods to treat chronic pain by rescuing the KCC2 expression deficit.

### BRIEF SUMMARY OF THE DISCLOSURE

The present disclosure is based, in part, on the discovery by the inventor that renormalizing inhibitory neurotransmission can be achieved by upregulating neuronal chloride-extruding transporter, KCC2 (SLC12A5), via GSK3ß inhibition and/or by use of a gene-therapeutic approach leveraging a novel signaling mechanism of delta-2 catenin.

One aspect of the present disclosure provides a recombinant transgene that includes a polynucleotide that encodes human-delta-catenin protein (e.g., SEQ ID NO:20), or a human-delta-catenin protein variant, including human-delta-catenin (S276A) protein (SEQ ID NO:32) or a fragment, isoform, or homologue thereof.

An aspect of the disclosure provides an expression cassette including a nucleotide sequence encoding the human-delta-catenin protein (e.g., SEQ ID NO:20), or a human-delta-catenin protein variant, including human-delta-catenin (S276A) protein (SEQ ID NO:32) or a fragment, isoform, or homologue thereof.

In some embodiments, the expression cassette further includes a nucleotide sequence that is codon-optimized to reduce CpG methylation sites and mammalian expression encoding the human-delta-catenin transgene. In some embodiments, the expression cassette includes a human-delta-catenin transgene sequence operably linked to a promoter and a polyadenylation sequence, which, in some embodiments, includes synapsin 1, calcium/calmodulin-dependent protein kinase II, tubulin alpha 1, neuron-specific enolase, human KCC2 promoter, or platelet-derived growth factor beta chain promoters.

In some embodiments, the expression cassette includes a constitutively active promoter, which, in some embodiments, includes human β-actin, human elongation factor-1α, chicken β-actin combined with cytomegalovirus early enhancer, cytomegalovirus (CMV), simian virus 40, and herpes simplex virus thymidine kinase. In some embodiments, the expression cassette includes a neuro-specific promoter.

In some embodiments, the expression cassette further includes a transcriptional termination signal, which signal can include bovine growth hormone polyadenylation signal (BGHpA), Simian virus 40 polyadenylation signal (SV40pA), and a synthetic polyadenylation signal.

An aspect of the disclosure provides a recombinant viral vector including a nucleotide sequence encoding the gene for human-delta-catenin (e.g., SEQ ID NO:20), or a human-delta-catenin protein variant, including human-delta-catenin (S276A) protein (SEQ ID NO:32), or a fragment, isoform, or homologue thereof. In some embodiments, the recombinant viral vector includes an expression cassette as described above and herein. In some embodiments, the recombinant viral vector further includes one or more of the following elements: (a) an inverted terminal repeat sequence (ITR); (b) a promoter; (c) an intron; (d) transcription terminator; and (e) a flanking inverted terminal repeat sequence (ITR).

In some embodiments of the recombinant viral vector that include a promoter, the promoter can include synapsin 1, calcium/calmodulin-dependent protein kinase II, tubulin alpha 1, neuron-specific enolase, human KCC2 promoter, or platelet-derived growth factor beta chain promoters. In some embodiments, the promoter is a constitutively active promoter, which, in some embodiments, can include human β-actin, human elongation factor-1a, chicken 3-actin combined with cytomegalovirus early enhancer, cytomegalovirus (CMV), simian virus 40, and herpes simplex virus thymidine kinase promoters. In some embodiments, the recombinant viral vector includes a neuro-specific promoter.

In some embodiments, the recombinant viral vector further includes a transcriptional termination signal, which can include bovine growth hormone polyadenylation signal (BGHpA), Simian virus 40 polyadenylation signal (SV40pA), and a synthetic polyadenylation signal.

In some embodiments, the recombinant viral vector can include adenoviruses, Adeno-associated viruses (AAV), Herpes simplex viruses (e.g., Herpes Simplex Virus Type 1), Retroviruses, lentiviruses, alphaviruses, flaviviruses, rhabdoviruses, measles virus, Newcastle disease virus, poxviruses, and picornaviruses. In some embodiments, the recombinant viral vector is an adeno-associated virus (AAV).

In some embodiments, the recombinant AAV vector can include a serotype of AAV1, AAV2, AAV4, AAV5, AAV6, AAV7, AAVrh74, AAV8, AAV9, AAV10, AAV11, AAV12, or AAV13.

In another aspect the disclosure provides compositions including a transgene as described above and herein, an expression cassette as described above and herein, or a recombinant viral vector as described above and herein. In some embodiments, the compositions are pharmaceutical compositions that include a pharmaceutically acceptable carrier and/or excipient.

In another aspect, the disclosure provides methods related to the transgenes, expression cassettes, recombinant viral vectors, recombinant AAV vectors, compositions and pharmaceutical compositions described above and herein. In some embodiments, methods of treating pain in a subject in need thereof are provided, including administering a therapeutically effective amount of a composition as described above and herein such that the pain is treated in the subject. In some embodiments, the pain is neuropathic pain or pathological pain. In some embodiments, methods of treating itch in a subject are provided, including administering to the subject a therapeutically effective amount of a composition described above and herein such that the itch is treated in the subject. In some embodiments, the itch is neuropathic or pathological itch.

In some embodiments, methods of increasing KCC2 mRNA levels in a subject are provided, including administering to the subject a therapeutically effective amount of a composition as described above and herein so that the KCC2 mRNA levels are increased in the subject compared to a pre-treatment baseline.

In some embodiments, methods of reducing intracellular chloride ion levels ([Cl—]i) in a central nervous system cell in a subject are provided, including administering to the subject a therapeutically effective amount of a composition as described above and herein so that the [Cl—]i in a central nervous system cell is reduced in the subject compared to a pre-treatment baseline.

In some embodiments, methods of increasing chloride ion efflux in a central nervous system cell in a subject are provided, including administering to the subject a therapeutically effective amount of a composition as described above and herein so that the chloride ion efflux is increased in the subject.

In some embodiments, methods of increasing synaptophysin expression in a central nervous system cell in a subject are provided, including administering to the subject a therapeutically effective amount of a composition as described above and herein so that the synaptophysin expression is increased in the subject as compared to a pre-treatment baseline.

In some embodiments, methods of increasing KCC2 expression in a subject are provided, including administering to the subject a therapeutically effective amount of a composition as described above and herein so that KCC2 expression is increased in the subject compared to pre-treatment baseline.

In some embodiments of the methods described above and herein, the methods further include administering to the subject a therapeutically effective amount of at least one additional compound, which compound, in some embodiments, includes a GSK3β inhibitor, an anti-analgesic, a muscle relaxant, an anti-anxiety drug, an antidepressant, an anticonvulsant, and combinations thereof. In some embodiments, the at least one additional compound includes a GSK3β inhibitor a corticosteroid, a counterirritant, an antihistamine, and a local anesthetic and combinations thereof.

In another aspect, the disclosure provides methods of treating pain and/or treating itch in a subject in need thereof, including administering a therapeutically effective amount of a GSK3β inhibitor, or a pharmaceutical composition including a GSK3β inhibitor and a pharmaceutically acceptable carrier and/or excipient, such that the pain is treated in the subject. In some embodiments, the pain and/or itch is neuropathic or pathological in nature.

In some embodiments, methods of increasing KCC2 mRNA levels in a subject are provided, including administering to the subject a therapeutically effective amount of a GSK3β inhibitor, or a pharmaceutical composition including a GSK3β inhibitor and a pharmaceutically acceptable carrier and/or excipient, so that the KCC2 mRNA levels are increased in the subject.

In some embodiments, methods of reducing intracellular chloride ion levels ([Cl—]i) in a central nervous system cell in a subject are provided, including administering to the subject a therapeutically effective amount of a GSK3β inhibitor, or a pharmaceutical composition including a GSK3β inhibitor and a pharmaceutically acceptable carrier and/or excipient, so that the [Cl—]i in a central nervous system cell is reduced in the subject.

In some embodiments, methods of increasing chloride ion efflux in a central nervous system cell in a subject are provided, including administering to the subject a therapeutically effective amount of a GSK3β inhibitor, or a pharmaceutical composition including a GSK3β inhibitor and a pharmaceutically acceptable carrier and/or excipient, so that the chloride ion efflux is increased in the subject.

In some embodiments, methods of increasing synaptophysin expression in a central nervous system cell in a subject in need thereof, comprising administering to the subject a therapeutically effective amount of a GSK3β inhibitor, or a pharmaceutical composition comprising a GSK3β inhibitor and a pharmaceutically acceptable carrier and/or excipient, such that the synaptophysin expression is increased in the subject.

In some embodiments, methods of increasing KCC2 expression in a subject are provided, including administering to the subject a therapeutically effective amount of a GSK3β inhibitor, or a pharmaceutical composition including a GSK3β inhibitor and a pharmaceutically acceptable carrier and/or excipient, so that KCC2 expression is increased in the subject.

In some embodiments of the methods described above, the methods further include administering to the subject a therapeutically effective amount of at least one additional compound, which compound, in some embodiments, includes compositions including a transgene, an expression cassette, or a recombinant viral vector as described above and herein (each, in some embodiments, pharmaceutical compositions that include a pharmaceutically acceptable carrier and/or excipient), an anti-analgesic, a muscle relaxant, an anti-anxiety drug, an antidepressant, an anticonvulsant, and combinations thereof. In some embodiments, the additional compound can include compositions including a transgene, an expression cassette, or a recombinant viral vector as described above and herein (each, in some embodiments, pharmaceutical compositions that include a pharmaceutically acceptable carrier and/or excipient), a corticosteroid, a counterirritant, an antihistamine, and a local anesthetic and combinations thereof.

In the methods described in all relevant aspects and embodiments of the disclosure, the central nervous system cell is a neuron.

In the methods described in all relevant aspects and embodiments of the disclosure, the composition can be administered by any administration route, including intrathecally, intra-cerebroventricularly, intra-cerebrally, perispinally, intra-spinally, intravenously and others.

### BRIEF DESCRIPTION OF THE DRAWINGS

FIG. 1 is a schematic of the compound screening for enhancers of KCC2 expression in primary cortical neurons that yielded kenpaullone. Screening paradigm using three rounds of primary screen based on luciferase (LUC) activity followed by secondary screen including KCC2 RT-qPCR and Clomeleon chloride imaging. Bottom panel: Four compound “winners” including kenpaullone (KP); screening conducted in primary mouse neurons.

FIGS. 2A-2F shows that kenpaullone enhances KCC2 gene expression and function in rat and human primary cortical neurons. FIG. 2A is a graph showing dose-dependent increase in KCC2 mRNA expression after KP treatment in primary rat cortical neurons. Results represent the average mRNA expression of multiple independent neuronal cultures, n=12 (control), n=4 (0.1 μM KP), n=3 (0.2 μM KP), n=10 (0.5 μM KP). *p<0.05, **p<0.01, ****p<0.0001, one-way ANOVA. FIG. 2B is a graph showing neuronal [Cl—]i, measured with ratiometric chloride indicator in primary rat cortical neurons, clomeleon, is robustly and significantly reduced after KP treatment. n≥75 neurons/3 independent cultures; **** p<0.0001, t-test. FIG. 2C is a graph showing that add-on treatment with KCC2-transport blocker, VU0240551 (2.5 μM), leads to a [Cl—]i≥120 mM, for both, vehicle-treated and KP-treated, indicating that KP's chloride lowering effect relies on KCC2 chloride extruding transport function. n≥75 neurons/3 independent cultures. FIG. 2D is a graph showing that kenpaullone does not enhance KCC2 chloride transporter mediated chloride efflux. Rat primary cortical neurons, n≥40 neurons per group. No difference was observed in neuronal chloride levels, measured with clomeleon fluorescent indicator after KP treatment vs vehicle at the 10 min and 20 min time-points. FIG. 2E is a graph showing KCC2 mRNA increases in a dose-dependent manner upon treatment with KP in primary human cortical neurons. Results represent the average mRNA expression of 3 independent neuronal cultures. *p<0.05, one-way ANOVA. FIG. 2F is a graph showing morphometry of immuno-cytochemistry of primary human cortical neurons shows increased KCC2 (62%; left-hand) and synaptophsin (54%; right-hand) expression after KP treatment, relative to vehicle. n=27-28 neurons analyzed for KCC2 ***p<0.001; n=17-18 neurons analyzed for synaptophysin, *p<0.01, t-test.

FIG. 3 shows that kenpaullone has an analgesic effect in inflammatory pain, evoked by peripheral tissue injection of CFA. Top Panel: Timeline for behavior assay. Middle Panel: Sensitized withdrawal thresholds in response to mechanical cues became significantly less sensitive. No significant difference in KP effects between low and high doses 10 mg/kg and 30 mg/kg. Bottom Panel: Thermal Pain threshold becomes significantly less sensitive only at day 7 after injury, for both low and high dose KP. *p<0.05 KP-treated vs vehicle treated, one-way ANOVA.

FIGS. 4A-4F shows that kenpaullone is analgesic in mouse nerve injury pain. FIG. 4A shows that systemic KP is analgesic for nerve injury pain. Top schematic: Overview of timeline of systemic injection and behavioral metrics. Mice were injected intraperitoneally (i.p) with either 10 mg/kg or 30 mg/kg KP daily after nerve constriction injury. Bar graphs: Analgesic effects of KP for sensitized mechanical withdrawal were dose-dependent, namely less accentuated at 10 mg/kg and more pronounced at 30 mg/kg. *p<0.05, **p<0.01, one-way ANOVA. FIG. 4B shows that intrathecal (i.t) KP is analgesic for nerve injury pain. Top schmatic: Overview of timeline of i.t injection and behavioral metrics. Bar graphs: analgesic effects of KP for sensitized mechanical withdrawal were observed upon daily i.t injection, note increasing sensitization in vehicle injected controls. Withdrawal behavior was measured at two times after i.t injection, +3 h, +24 h. *p<0.05, **p<0.01, one-way ANOVA. FIG. 4C shows that i.t co-application of KP and specific KCC2 transport inhibitor compound, VU0240551, eliminates the central analgesic effects of KP. Behavior assays were conducted at 3 h, 4 h and 5 h on day 7 after i.t injection. *p<0.05, **p<0.01, one-way ANOVA. FIG. 4D shows that kenpaullone is anti-pruritic. Top schematic: Timeline for DNFB contact sensitization and behavioral assays. Bottom, bar graph: In a DNFB chronic contact dermatitis model, daily intraperitoneal injections of KP (30 mg/kg) significantly reduced robust scratching behavior of sensitized sites. n=8-10 mice per group. * p<0.01 KP-treated vs vehicle, t-test. FIG. 4E is a graph showing the effect of KP on motor function and coordination of mice—rotarod assay. Bar diagram showing the mean of elapsed time on the rotarod. KP does not affect motor stamina and coordination in mice, with the one-time exception of high-dose KP on day 7. n=3-4 mice per group. FIG. 4F shows that KP does not evoke conditioned place preference (CPP). The timeline for the CPP assay is shown on top. Bottom graph: No significant difference in CPP scores between KP and vehicle (t-test, p>0.05, n=5 mice per group).

FIGS. 5A-5D shows that kenpaullone re-normalizes E_{GABA} in spinal cord dorsal horn by increasing KCC2 expression/function. FIG. 5A top left is a schematic of the Lamina-I/II area of spinal cord dorsal horn (SCDH) is highlighted. Top right: image representation of lamina-I/II area before and after laser capture microdissection. Bottom, bar graphs showing KP (10 mg/kg) rescues significantly attenuated KCC2 mRNA expression in the SCDH after nerve constriction injury (PSNL) vs vehicle treated mice; n=4 mice/group, **** p<0.0001, one-way ANOVA. FIG. 5B top panels show representative KCC2 immuno-staining of the SCDH in nerve constriction injury and CFA-mediated inflammatory injury. Bottom, bar graphs show KP (10 mg/kg) increases KCC2 protein expression in SCDH compared to vehicle control in nerve constriction injury and inflammatory pain models. n=5 mice/group, #p<0.01 vs IFA or sham PSNL; **p<0.01 KP vs vehicle; one-way ANOVA. FIG. 5C is a graph showing potent behavioral sensitization of juvenile mice after peripheral nerve constriction injury (PSNL) and its almost complete behavioral recovery after treatment with KP (30 mg/kg). n=3 mice sham n=4 mice PSNL+vehicle, n=3 mice PSNL+KP. Note accentuated responses for sensitization and rescue in younger mice vs adult mice (compare with FIG. 4A). FIG. 5D shows that spinal cord slices were isolated from the same juvenile mice as shown in FIG. 5C, and lamina-II neurons were examined for E_{GABA} using the perforated patch method, as illustrated by the schematic, 1-3 neurons per mouse. The left-hand panel shows a representative I-V plot. The right-hand, bar graph shows quantification of E_{GABA} indicating a significant depolarizing shift in sham vs PSNL with vehicle treatment (“PSNL”). Note significant hyperpolarization in response to KP treatment in PSNL mice. Of note, PSNL plus KP was not different from sham injury. n=4 neurons (sham), n=9 neurons (PSNL), n=6 neurons (PSNL+KP); ****p<0.0001, one-way ANOVA.

FIGS. 6A-6B shows electrophysiological recordings from spinal cord dorsal horn. FIG. 6A are current responses of layer-II neurons to a voltage ramp from +8 to −92 mV in control (grey traces, obtained before GABA puff), or at the end of a puff of GABA (black trace) in sham, PSNL+vehicle and PSNL+KP groups. Reversal potential of the GABA-evoked current is at the voltage where the grey and black traces intersect (arrow). FIG. 6B is a voltage ramp from +8 to −92 mV.

FIG. 7 is a graph showing kenpaullone increases KCC2 expression in central neurons by inhibiting GSK3ß, not CDKs. GSK3-inhibitors increase KCC2 mRNA expression, measured by RT-qPCR, in a dose-dependent manner, whereas several CDK-inhibitors do not increase KCC2 mRNA expression, or even reduce it. Rat primary cortical neurons. Results represent the average mRNA expression of 3-6 independent neuronal cultures. **p<0.01, ****p<0.0001, compound vs vehicle, one-way ANOVA.

FIGS. 8A-8B shows the cellular mechanism of action of kenpaullone in central neurons. FIG. 8A is a schematic showing DARTS methodology to identify proteins that bind to KP in rat primary cortical neurons. The chart shows KP binding to GSK3β is independent of detergent treatment of the protein sample preparation from the neuronal culture. Binding to GSK3ß was documented whereas binding to CDKs was not. FIG. 8B is a chart showing that phosphoproteomics assays reveal S259 phosphorylation target in δ-cat protein after KP treatment of rat primary cortical neurons, significant de-phosphorylation resulted after 1 h treatment and was sustained at 24 h. The peptide sequence represents amino acids 298-315 of rat δ-catenin (F1M787; SEQ ID NO:19). The schematic is a representation of structure of human δ-cat (CTNND2) showing functional domains. Human residue S276 matches rat S259. The Armadillo domain region plays a key role in transcription factor Kaiso binding to δ-cat. FIG. 8C is a graph showing that KP significantly increases δ-cat nuclear translocation. n=33-34 neurons/group,****p<0.001 KP vs vehicle, t-test. FIG. 8D is a graph showing KP significantly enhances nuclear transfer of δ-cat when transfected with δ-cat(WT). Mutation δ-cat(S276A) increases nuclear transfer, but treatment with KP had no effect. n=25-30 neurons/group ****p<0.0001 vs vehicle, mixed effects statistics.

FIG. 9 is a graph showing that KP significantly increases S-catenin translocation into the nucleus; n=18 neurons/group.

FIG. 10 is a graph showing that inhibiting δ-catenin/TCF mediated transcription attenuates KCC2 expression. Rat primary cortical neurons, 4 independent cultures were used. Catenin-DNA interaction inhibitor ICG-001 significantly decreases KCC2 mRNA expression in a dose-dependent manner. Note opposite effect of KP which significantly increases KCC2 mRNA expression. #p<0.05 increase, *p<0.05 decrease vs vehicle, one-way ANOVA.

FIGS. 11A-11D shows that kenpaullone regulates KCC2 promoter activity via S-cat and two Kaiso sites. FIG. 11A shows a schematic on the top panel of the structure of mouse KCC2 gene encompassing 2.5 kb surrounding the transcription start site (TSS; +1). Location of DNA binding sites: Kaiso1 (−1456 to −1449), Kaiso2 (+83 to +90) and TCF (−1845 to −1838) relative to TSS, all three sites can bind delta-cat via Kaiso (Kaiso1, 2 sites) and beta-cat (TCF). Bottom, bar graph: Chromatin immuno-precipitation (ChIP) using anti-delta-cat antibody in rat primary cortical neurons reveals binding of delta-cat to all three sites. KP treatment significantly increases binding of delta-cat to the KCC2 promoter on the Kaiso2 binding site, significantly reduced binding to Kaiso1, and non-significant increase at TCF. n=6 independent neuronal cultures were subjected to ChIP; *p<0.05 KP-treatment vs vehicle, t-test. FIG. 11B shows that KP increases beta-cat binding to the TCF site in the KCC2 promoter. Upper panel: Schematic of the structure of mouse-KCC2 gene encompassing 2.5 kb surrounding the TSS (+1), as in FIG. 11A. Bottom, bar graph: Chromatin immuno-precipitation using anti-beta-cat antibody in primary rat cortical neurons reveals binding of beta-cat to TCF, to minor degree to Kaiso1, 2. KP treatment significantly increases binding of beta-cat to the KCC2 promoter on the TCF binding site. n=6 independent rat primary neuron cultures were subjected to ChIP, **p<0.01 t-test, KP-treated vs vehicle. FIG. 11C shows the Relevance of TCF DNA binding site on regulation of KCC2 [symbol] LUC. N2a differentiated cells were used. Top panel is a schematic of the mouse KCC2 promoter constructs, as in FIG. 11D. Bottom, bar graph: KP did not increase promoter activity for any of the constructs. Triple-deletion of TCF and both Kaiso sites rendered the KCC2 promoter very low in activity, compared with WT (without KP), at <⅓ of its activity, which is less than the ΔΔKaiso1,2 deletion construct (FIG. 11D; <½). n=3 independent cultures, **** p<0.0001 WT vs triple-deletion construct, 1-way ANOVA. FIG. 11D shows a schematic on the top panel of the mouse KCC2 promoter constructs, Kaiso1, -2 were deleted and a ΔK1/K2 construct was built devoid of both sites. Dual-RE1 sites and TCF site shown for orientation, also TSS at +1. Bottom, bar graph: Luciferase (LUC) activity of KCC2 promoter constructs in N2a cells with neuronal differentiation. Left-hand: ΔK1 significantly increased over WT, ΔK2 and ΔK1/K2 significantly decreased vs WT, n=3 independent culture per transfection, *p<0.05, 1-way ANOVA. Right-hand: repressive Kaiso1 binding site deletion with enhanced activity of the KCC2 promoter, significantly enhanced further in response to KP. n=3 independent cultures per transfection; ***p<0.001 KP-treatment vs vehicle for the respective construct, t-test.

FIGS. 12A-12D shows that δ-cat spinal transgenesis is analgesic in nerve constriction injury. FIG. 12A is a graph δ-cat(WT) transgene significantly increases KCC2 mRNA expression in differentiated N2a cells, and expression level was slightly elevated when transfecting δ-cat(S276A), both significantly increased over WT. n=3 independent cultures were subjected to transfection with the δ-cat constructs; **p<0.01 δ-cat construct vs control transfection, one-way ANOVA. FIG. 12B is a schematic showing a timeline for behavioral testing after constriction nerve injury and subsequent i.t injection of AAV9 transgenesis vectors. FIG. 12C is a graph showing mechanical withdrawal thresholds after nerve constriction injury (PSNL). Significant improvement of sensitization for both S-cat(WT) and δ-cat(S276A) constructs with sustained benefit over 2 weeks. Note more potent analgesia by δ-cat(S276A) based on the data on d7, d14. n=5-6 mice/tdTomato ctrl, n=7 mice/δ-cat transgenes; *p<0.05 **p<0.01, mixed effects statistics. FIG. 12D is a graph showing KCC2 mRNA in microdissected SCDH was significantly increased in δ-cat(S276A) as assessed by RT-qPCR. For S-cat(WT), KCC2 abundance was elevated but not to significant levels likely because of viral transduction of only a fraction of sensory relay neurons in the SCDH. n=3 mice/group, * p<0.05 δ-cat(S276A) vs tdTomato control, one-way ANOVA.

FIG. 13 is a schematic showing the analgesic mechanism of action of kenpaullone. Left panel: Nerve injury facilitates activation of GSK3β kinase in pain relay neurons in layer-II of the SCDH. GSK3ß phosphorylates δ-cat in the cytoplasm at S259/S276. Phospho-S-cat is unstable, undergoes ubiquitination and subsequent degradation. In the nucleus, Kaiso recruits repressive transcription factors on the KCC2 gene promoter which leads to overall repressed KCC2 transcription. Right panel: Treatment with KP inactivates GSK3β in SCDH layer-II pain relay neurons. In turn, this leads to de-phosphorylation and stabilization of δ-cat which enters the nucleus. In the nucleus, δ-cat binds to Kaiso transcription factor complex and displaces repressive transcription factors. This leads to a net enhancement of KCC2 expression.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “nucleic acid” refers to isolated, purified, natural, recombinant, synthetic deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the reference sequence explicitly indicated.

As used herein, “nucleic acid therapy” or “gene therapy” refers to the transfer or insertion of nucleic acid molecules into certain cells, which may be referred to as target cells, to produce specific gene products that are involved in correcting or modulating diseases or disorders and/or promote beneficial biological processes. The nucleic acid is introduced into the selected target cells in a manner such that the nucleic acid is expressed and a product encoded thereby is produced. Alternatively, the nucleic acid may in some manner mediate the expression of nucleic acid that encodes a therapeutic product. This product may be a therapeutic compound, which is produced in therapeutically effective amounts or at a therapeutically useful time. It may also encode a product, such as a peptide or RNA, that in some manner mediates, directly or indirectly, expression of a therapeutic product. Expression of the nucleic acid by the target cells within an organism afflicted with a disease or disorder thereby provides a way to modulate the disease or disorder or beneficial biological processes. The nucleic acid encoding the therapeutic product may be modified prior to introduction into the target cell in order to enhance or otherwise alter the product or expression thereof. Nucleic acid therapy also refers to administration or in situ generation of a nucleic acid or a derivative thereof which specifically hybridizes (e.g., binds) under cellular conditions with the cellular mRNA and/or genomic DNA encoding one of a target polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

For use in nucleic acid therapy, cells can be transfected in vitro via methods of the disclosure, followed by introduction of the transfected cells into the body of a subject. This is often referred to as ex vivo nucleic acid therapy. Alternatively, the cells can be transfected directly in vivo within the body of a subject.

As used herein, “heterologous” or “foreign” with reference to nucleic acids, DNA and RNA are used interchangeably and refer to nucleic acid, DNA or RNA that does not occur naturally as part of the genome in which it is present or which is found in a location(s) or in an amount in the genome that differs from that in which it occurs in nature. It is nucleic acid that has been exogenously introduced into the cell. Thus, heterologous nucleic acid is nucleic acid not normally found in the host genome in an identical context. Examples of heterologous nucleic acids include, but are not limited to, DNA that encodes a gene product or gene product(s) of interest, introduced for purposes of gene therapy or for production of an encoded protein. Other examples of heterologous DNA include, but are not limited to, DNA that encodes a selectable marker, DNA that encodes therapeutically effective substances, such as anti-pain or anti-itch agents, enzymes and hormones, and DNA that encodes other types of proteins, such as antibodies.

As used herein, the term “promoter” refers to a DNA regulatory region capable of binding RNA polymerase in a mammalian cell and initiating transcription of a downstream (3′ direction) coding sequence operably linked thereto. For purposes of the present disclosure, a promoter sequence includes the minimum number of bases or elements necessary to initiate transcription of a gene of interest at levels detectable above background. Within the promoter sequence may be a transcription initiation site, as well as protein binding domains (consensus sequences) responsible for the binding of RNA polymerase. Eukaryotic promoters will often, but not always, contain “TATA” boxes and “CAT” boxes. Promoters include those that are naturally contiguous to a nucleic acid molecule and those that are not naturally contiguous to a nucleic acid molecule. Additionally, the term “promoter” includes inducible promoters, conditionally active promoters such as a cre-lox promoter, constitutive promoters, and tissue specific promoters. Examples of suitable promoter include, but are not limited to, synapsin 1, calcium/calmodulin-dependent protein kinase II, tubulin alpha 1, neuron-specific enolase, human KCC2 promoter, platelet-derived growth factor beta chain promoters and the like.

As used herein, “transformation” or “transfection” or “transfected” refers to the process by which nucleic acids are introduced into cells with or without the use of one or more accompanying facilitating agents such as lipofectamine. Transfection refers to the taking up of exogenous nucleic acid, by a host cell whether or not any coding sequences are in fact expressed. Methods and compositions of the disclosure are effective for transformation or transfection. Successful transfection is generally recognized by detection of the presence of the heterologous nucleic acid within the transfected cell, such as, for example, any visualization of the heterologous nucleic acid or any indication of the operation of a such nucleic acid within the host cell. Methods for transfection that are known in the art include, e.g., calcium phosphate transfection, DEAE dextran transfection, protoplast fusion, electroporation, and lipofection.

As used herein, the term “expression” refers to the conversion of the information contained in the nucleic acid molecule into a gene product. The gene product can be the direct transcriptional product of a gene (e.g., mRNA, tRNA, rRNA, antisense RNA, ribozyme, structural RNA, or any other type of RNA) or a peptide or polypeptide produced by translation of an mRNA. Gene products also include RNAs that are modified by processes such as capping, polyadenylation, methylation, and editing; and proteins modified by, for example, methylation, acetylation, phosphorylation, ubiquitination, ADP-ribosylation, myristilation, and glycosylation.

As used herein, the term “host cell” refers to an individual cell or a cell culture that can be or has been a recipient of any recombinant vector(s) or isolated polynucleotide(s). Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell that comprises a recombinant vector of the invention may be called a “recombinant host cell.”

As used herein, the term “recombinant,” with respect to a nucleic acid molecule, means a polynucleotide of genomic, cDNA, viral, semisynthetic, and/or synthetic origin which, by virtue of its origin or manipulation, is not associated with all or a portion of the polynucleotide with which it is associated in nature. The term “recombinant”, as used with respect to a protein or polypeptide, means a polypeptide produced by expression of a recombinant polynucleotide. The term “recombinant” as used with respect to a host cell means a host cell into which a recombinant polynucleotide has been introduced.

As used herein, the phrase “recombinant virus” and “recombinant viral vector” are used interchangeable and refer to a virus or viral vector that is genetically modified by the hand of man. The phrase covers any virus known in the art. In some embodiments, the recombinant viral vector is selected from the group consisting of adenoviruses, Adeno-associated viruses (AAV), Herpes simplex viruses (e.g., Herpes Simplex Virus Type 1), Retroviruses, lentiviruses, alphaviruses, flaviviruses, rhabdoviruses, measles virus, Newcastle disease virus, poxviruses, and picornaviruses. As used herein, the term “vector” refers to an agent (e.g., a plasmid or virus) used to transmit genetic material to a host cell or organism. A vector may be composed of either DNA or RNA. In some embodiments, the recombinant viral vector comprises a recombinant AAV vector. In other embodiments, recombinant AAV vector comprises a serotype selected from the group consisting of AAV1, AAV2, AAV4, AAV5, AAV6, AAV7, AAVrh74, AAV8, AAV9, AAV10, AAV 11, AAV12, or AAV13. In some embodiments, the recombinant AAV vector is selected from the group consisting of AAV1, AAV8, and AAV9. In certain embodiments, the recombinant AAV vector comprises AAV9. In other embodiments, the virus comprises a lentivirus.

As used herein, the term “biologically active” entity, or an entity having “biological activity,” is one having structural, regulatory, or biochemical functions of a naturally occurring molecule or any function related to or associated with a metabolic or physiological process. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, such as hybridization, when it has therapeutic value in alleviating a disease condition, when it has prophylactic value in inducing an immune response, when it has diagnostic and/or prognostic value in determining the presence of a molecule, such as a biologically active fragment of a polynucleotide that can, for example, be detected as unique for the polynucleotide molecule, or that can be used as a primer in a polymerase chain reaction. A biologically active polypeptide or fragment thereof includes one that can participate in a biological reaction.

As used herein, the terms “polypeptide” and “protein” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, a “polypeptide” may refer to a protein which includes modifications, such as deletions, additions, and substitutions (generally conservative in nature), to the native sequence, as long as the protein maintains the desired activity. These modifications may be deliberate or may be accidental.

As described herein, “sequence identity” is related to sequence homology. Homology comparisons may be conducted by eye or using sequence comparison programs. These commercially available computer programs may calculate percent (%) homology between two or more sequences and may also calculate the sequence identity shared by two or more amino acid or nucleic acid sequences. Sequence homologies may be generated by any of a number of computer programs known in the art, for example BLAST or FASTA.

Percentage (%) sequence identify can be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in percent homology when a global alignment is performed. Therefore, most sequence comparison methods are designed to produce optimal alignments that take into consideration possible insertions and deletions without unduly penalizing the overall homology or identity score. This is achieved by inserting “gaps” in the sequence alignment to try to maximize local homology or identity.

As used herein, “administration” of a disclosed compound encompasses the delivery to a subject of a compound as described herein, or a prodrug or other pharmaceutically acceptable derivative thereof, using any suitable formulation or route of administration, as discussed herein.

As used herein, the terms “effective amount” or “therapeutically effective amount” refer to that amount of a compound, transgene, and any pharmaceutical compositions thereof and described herein that is sufficient to effect the intended application including, but not limited to, disease treatment, as illustrated below. In some embodiments, the amount is that effective for detectable reduction of pain or itch. In some embodiments, the amount is that effective for alleviating, reducing or eliminating a pathologic pain or itch condition.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

For a recombinant viral vector, e.g., rAAV, an effective amount is also an amount sufficient to infect a sufficient number of cells of a target tissue in a subject. In some cases, an effective amount of a rAAV may be an amount sufficient to produce a stable somatic transgenic animal model. As an example regarding the effects of route of administration, targeting a CNS tissue by intravascular injection may require different (e.g., higher) doses, in some cases, than targeting CNS tissue by intrathecal or intracerebral injection. In some cases, multiple doses of a rAAV may be administered.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human. In other embodiments, the subject comprises a human suffering from pain and/or itch.

As used herein, the term “disease” and “disorder” are used interchangeably and refer to any disorder or structure or function is a human, animal, or plant, especially one that produces signs or symptoms or that affects a specific location and is not simply a direct result of physical injury. Examples include, but are not limited to, neurological disorders, pain, itch, metabolic disorders, cancer, genetic disorders, and the like.

As used herein, the term “pain” refers to the basic bodily sensation induced by a noxious stimulus, received by naked nerve endings, characterized by physical discomfort (e.g., pricking, throbbing, aching, etc.) and typically leading to an evasive action by the individual. As used herein, the term pain also includes chronic and acute neuropathic pain as well as pathologic pain. The terms “neuropathic pain” or “neurogenic pain” can be used interchangeable and refer to pain that arises from direct stimulation of nervous tissue itself, central or peripheral and can persist in the absence of stimulus. The sensations that characterize neuropathic pain vary and are often multiple and include burning, gnawing, aching, and shooting. (See Rooper and Brown, (2005) Adams and Victor's Principles of Neurology, 8th ed., NY, McGraw-Hill). These damaged nerve fibers send incorrect signals to other pain centers. The impact of nerve fiber injury includes a change in nerve function both at the site of injury and areas around the injury. Chronic neuropathic pain often seems to have no obvious cause; however, some common causes may include, but are not limited to, alcoholism, amputation, back, leg and hip problems, chemotherapy, diabetes, facial nerve problems, HIV infection or AIDS, multiple sclerosis, shingles, and spine surgery. For example, one example of neuropathic pain is phantom limb syndrome, which occurs when an arm or leg has been removed because of illness or injury, but the brain still gets pain messages from the nerves that originally carried impulses from the missing limb. The term “pathologic pain” refers to that pain that is characterized by an amplified response to normally innocuous stimuli, and an amplified response to acute pain. Pain as used herein may also refer to both chronic and acute pain. Suitable examples of pain include, but are not limited to, pain of osteoarthritis, cancer pain, chronic low back pain, low back pain of osteoporosis, pain of bone fracture, pain of rheumatoid arthritis, neuropathic pain, postherpetic pain, pain of diabetic neuropathy, fibromyalgia, pain of pancreatitis, pain of interstitial cystitis, pain of endometriosis, pain of irritable bowel syndrome, migraine, postoperative pain, pain of pulpitis and the like.

As used herein, the terms “itch,” “pruritus” and its alternative spelling “pruritis” are used interchangeably and refer to those irritating skin sensations that provoke a desire to scratch. Pruritus may range from mildly unpleasant and temporary to acute and persistent sensations. A number of skin (e.g., fungal infections, and skin conditions such as atopic dermatitis) and systemic conditions (e.g., renal failure, liver damage, liver disease (e.g., cirrhosis), acquired immune deficiency syndrome (AIDS), polycythemia vera, diabetes, hyperthyroidism, and cancer (e.g., Hodgkin's lymphoma, non-Hodgkin's lymphoma, and Kaposi's sarcoma) may be associated with acute and/or chronic pruritus, which can significantly reduce quality of life. Other causes of pruritus may include induction by cytokines or treatments such as chemotherapy and kidney dialysis. Suitable examples of pruritus may include, but are not limited to, systemic cutaneous pruritus, localized cutaneous pruritus, senile cutaneous pruritus, gestational pruritus, pruritus ani, vulvar pruritus and the like.

As used herein, the term “expression cassette” refers to a component of vector DNA consisting of a gene and regulatory sequence to be expressed by a transfected cell. In each successful transformation, the expression cassette directs the cell's machinery to make RNA and protein(s).

As used herein, the term “central nervous system cell” refers to neurons.

Unless otherwise defined, all technical terms used herein have the same meaning as commonly understood by one of ordinary skill in the art to which this disclosure belongs.

Compositions

The present disclosure is based, in part, on the discovery by the inventor that renormalizing inhibitory neurotransmission can be achieved by upregulating neuronal chloride-extruding transporter, KCC2 (SLC12A5), via GSK3ß inhibition and/or by use of a gene-therapeutic approach leveraging a novel signaling mechanism of delta-2 catenin.

Accordingly, one aspect of the disclosure provides a recombinant transgene that includes a polynucleotide that encodes human-delta-catenin protein, or a variant thereof. In some embodiments, the polynucleotide can comprise SEQ ID NO:20, SEQ ID NO:22, SEQ ID NO:24, SEQ ID NO:26, SEQ ID NO:28, or SEQ ID NO:30, or a sequence having at least 50%, 60%, 65%, 70%, 75%, 80%, 85%, 90%, 95%, 96%, 97%, 98% or 99% sequence identity to the sequence set forth in SEQ ID NO:20, SEQ ID NO:22, SEQ ID NO:24, SEQ ID NO:26, SEQ ID NO:28, or SEQ ID NO:30. In some embodiments, the polynucleotide encodes human-delta-catenin (S276A), or a variant thereof including polynucleotides that code for conservative substitutions of alanine, such as glycine (S276G), valine (S276V), leucine (S276L) or isoleucine (S276I). In some embodiments, the polynucleotide comprises SEQ ID NO:32, and in some embodiments, the polynucleotide include any that codes for the polypeptide of SEQ ID NO:33, or a sequence having at least 50%, 60%, 65%, 70%, 75%, 80%, 85%, 90%, 95%, 96%, 97%, 98% or 99% sequence identity to the sequence set forth in SEQ ID NO:32 and SEQ ID NO:33.

Another aspect of the disclosure provides a nucleic acid expression cassette that includes a nucleic acid sequence encoding the human delta-catenin protein, or a variant thereof. In some embodiments, the nucleic acid sequence in the expression cassette includes SEQ ID NO:20, SEQ ID NO:22, SEQ ID NO:24, SEQ ID NO:26, SEQ ID NO:28, or SEQ ID NO:30, or a sequence having at least 50%, 60%, 65%, 70%, 75%, 80%, 85%, 90%, 95%, 96%, 97%, 98% or 99% sequence identity to the sequence set forth in SEQ ID NO:20, SEQ ID NO:22, SEQ ID NO:24, SEQ ID NO:26, SEQ ID NO:28, or SEQ ID NO:30. In some embodiments, the nucleic acid sequence encodes human-delta-catenin (S276A), or a variant thereof including polynucleotides that code for conservative substitutions of alanine, such as glycine (S276G), valine (S276V), leucine (S276L) or isoleucine (S276I). In some embodiments, the polynucleotide includes SEQ ID NO:32 or a sequence having at least 50%, 60%, 65%, 70%, 75%, 80%, 85%, 90%, 95%, 96%, 97%, 98% or 99% sequence identity to the sequence set forth in SEQ ID NO:32.

In some embodiments, the expression cassette further includes a nucleotide sequence that is codon-optimized to reduce CpG methylation sites and mammalian expression encoding the human-delta-catenin transgene.

In some embodiments, the cassette comprises the human delta-catenin protein operably linked to a promoter and a polyadenylation sequence. Any promoter suitable for the expression of human-delta-catenin protein may be used. Examples include, but are not limited to, synapsin 1, calcium/calmodulin-dependent protein kinase II, tubulin alpha 1, neuron-specific enolase, human KCC2 promoter, platelet-derived growth factor beta chain promoters, and the like.

In some embodiments, the promoter includes a constitutively active promoter. In some embodiments, the constitutively active promoter can be, for example, human β-actin, human elongation factor-1a, chicken 3-actin combined with cytomegalovirus early enhancer, cytomegalovirus (CMV), simian virus 40, and herpes simplex virus thymidine kinase. In some embodiments, the expression cassette includes a neuro-specific promoter.

In some embodiments, the nucleic acid expression cassette further includes a transcriptional termination signal selected from the group consisting of bovine growth hormone polyadenylation signal (BGHpA), Simian virus 40 polyadenylation signal (SV40pA), and a synthetic polyadenylation signal.

Some embodiments of recombinant transgenes, expression cassettes, recombinant viral vectors and recombinant AAV vectors of the disclosure further include a transcriptional termination signal selected from the group consisting of bovine growth hormone polyadenylation signal (BGHpA), Simian virus 40 polyadenylation signal (SV40pA), and a synthetic polyadenylation signal.

Another aspect of the present disclosure provides compositions including a recombinant transgene that includes a polynucleotide that encodes human-delta-catenin protein, or a variant thereof as described herein. In some embodiments, the polynucleotide can comprise SEQ ID NO:20, SEQ ID NO:22, SEQ ID NO:24, SEQ ID NO:26, SEQ ID NO:28, or SEQ ID NO:30, or a sequence having at least 50%, 60%, 65%, 70%, 75%, 80%, 85%, 90%, 95%, 96%, 97%, 98% or 99% sequence identity to the sequence set forth in SEQ ID NO:20, SEQ ID NO:22, SEQ ID NO:24, SEQ ID NO:26, SEQ ID NO:28, or SEQ ID NO:30. In some embodiments, the nucleic acid sequence encodes human-delta-catenin (S276A), or a variant thereof including polynucleotides that code for conservative substitutions of alanine, such as glycine (S276G), valine (S276V), leucine (S276L) or isoleucine (S276I). In some embodiments, the polynucleotide includes SEQ ID NO:32 or a sequence having at least 50%, 60%, 65%, 70%, 75%, 80%, 85%, 90%, 95%, 96%, 97%, 98% or 99% sequence identity to the sequence set forth in SEQ ID NO:32.

In another aspect, the disclosure provides compositions that include an expression cassette that includes a nucleic acid sequence encoding the human delta-catenin protein, or a variant thereof, as described herein. In some embodiments, the nucleic acid sequence in the expression cassette includes SEQ ID NO:20, SEQ ID NO:22, SEQ ID NO:24, SEQ ID NO:26, SEQ ID NO:28, or SEQ ID NO:30, or a sequence having at least 50%, 60%, 65%, 70%, 75%, 80%, 85%, 90%, 95%, 96%, 97%, 98% or 99% sequence identity to the sequence set forth in SEQ ID NO:20, SEQ ID NO:22, SEQ ID NO:24, SEQ ID NO:26, SEQ ID NO:28, or SEQ ID NO:30. In some embodiments, the nucleic acid sequence encodes human-delta-catenin (S276A), or a variant thereof including polynucleotides that code for conservative substitutions of alanine, such as glycine (S276G), valine (S276V), leucine (S276L) or isoleucine (S276I). In some embodiments, the polynucleotide includes SEQ ID NO:32 or a sequence having at least 50%, 60%, 65%, 70%, 75%, 80%, 85%, 90%, 95%, 96%, 97%, 98% or 99% sequence identity to the sequence set forth in SEQ ID NO:32.

In another aspect, the disclosure provides compositions that include a recombinant viral vector as described below and herein. In some embodiments, the recombinant viral vector is an Adeno-associated virus as described below and herein.

Another aspect of the present disclosure provides a pharmaceutical composition including a composition according to the present disclosure and a pharmaceutically acceptable carrier and/or excipient.

Recombinant Viral Vectors

The recombinant transgenes and/or nucleic acid expression cassettes (e.g., human-delta-catenin and variants thereof) provided herein may be incorporated into recombinant viral vectors/viruses. Any suitable recombinant viral vector suitable for gene therapy is suitable for use in the compositions and methods according to the present disclosure. In some embodiments, the recombinant viral vector can include adenoviruses, Adeno-associated viruses (AAV), Herpes simplex viruses (e.g., Herpes Simplex Virus Type 1), Retroviruses, lentiviruses, alphaviruses, flaviviruses, rhabdoviruses, measles virus, Newcastle disease virus, poxviruses, or picornaviruses. In some embodiments, the recombinant viral vector includes a recombinant Adeno-Associated Viruses (AAV). In other embodiments, the recombinant viral vector comprises a recombinant lentiviral vector.

In some embodiments, the recombinant viral vectors according to the disclosure can include one or more of the following elements: an Inverted Terminal Repeat sequence (ITR), a promoter (e.g., a neuron-specific promoter), an intron, a (trans)gene (e.g., a transgene encoding human-delta-catenin, a variant thereof, a fragment thereof, an isoform thereof, or a homologue thereof), a transcription terminator, e.g., a polyadenylation signal, a flanking Inverted Terminal Repeat sequence (ITR).

In some embodiments, the promoter can include a synapsin 1 promoter, calcium/calmodulin-dependent protein kinase II promoter, tubulin alpha 1 promoter, neuron-specific enolase promoter, platelet-derived growth factor beta chain promoters, human KCC2 promoter, and the like. In one embodiment, the promoter comprises the human synapsin promoter. In another embodiment, the promoter comprises the human KCC2 promoter.

In some embodiments, the recombinant viral vector includes a constitutively active promoter. In some embodiments, the constitutively active promoter can be, for example, human β-actin, human elongation factor-1α, chicken β-actin combined with cytomegalovirus early enhancer, cytomegalovirus (CMV), simian virus 40, and herpes simplex virus thymidine kinase. In some embodiments, the recombinant viral vector includes a neuro-specific promoter.

In some embodiments, the recombinant viral vector further includes a transcriptional termination signal that can be, for example, bovine growth hormone polyadenylation signal (BGHpA), Simian virus 40 polyadenylation signal (SV40pA), and a synthetic polyadenylation signal.

In some embodiments the recombinant viral vector comprises Adeno-associated virus.

Recombinant AAV genomes of the present disclosure comprise nucleic acid molecule of the present disclosure (e.g., human-delta-catenin and variants thereof) and one or more AAV ITRs flanking a nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12 and AAV-13. In some embodiments, the AAV serotype used comprises AAV1, AAV8 and AAV9. In certain embodiments, the recombinant AAV comprises AAV9, and in certain embodiments, AAV-1. Production of pseudotyped rAAV are disclosed in, for example, WO 01/83692. Other types of rAAV variants, for example rAAV with capsid mutations, are also contemplated. (See, for example, Marsic et al., Molecular Therapy, 22(11): 1900-1909 (2014). It is understood that the nucleotide sequences of the genomes of various AAV serotypes are known in the art.

In some embodiments, the DNA plasmids of the present disclosure comprise rAAV genomes of the present disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety. In some embodiments, the recombinant AAV vector is selected from the group consisting of AAV1, AAV8, and AAV9 and expressing a nucleotide sequence encoding the human-delta-catenin protein and variants thereof.

A method of generating a packaging cell is to create a cell line that stably expresses all the necessary components for viral (e.g., AAV) particle production. For example, in one embodiment, a plasmid (or multiple plasmids) comprising a viral rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The present disclosure further provides packaging cells that produce infectious recombinant viral vectors (e.g. rAAV). In one embodiment, packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In embodiments comprising a recombinant AAV, the recombinant AAV (i.e., infectious encapsidated rAAV particles) of the present disclosure comprises a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

The recombinant viral vectors (e.g., rAAV) may be purified by methods standard in the art such as by column chromatography or cesium chloride gradients. Methods for purifying recombinant viral vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

Pharmaceutical Compositions

In another aspect, the present disclosure provides pharmaceutical compositions comprising a recombinant viral vector comprising the human-delta-catenin transgene and/or nucleic acid expression cassettes comprising the human-delta-catenin protein as described herein. Other pharmaceutical compositions contemplated in the present disclosure include those comprising GSK3β inhibitors. In some embodiments, compositions of the present disclosure include a recombinant viral vector and/or a pharmaceutically acceptable carrier and/or excipient. In other embodiments, compositions of the present disclosure include a GSK3β inhibitor as provided herein and/or a pharmaceutically acceptable carrier and/or excipient.

Pharmaceutically acceptable compositions are prepared in view of approvals for a regulatory agency or other agency prepared in accordance with generally recognized pharmacopeia for use in animals and in humans. The compounds can be formulated into any suitable pharmaceutical preparations for any of injectable, oral or topical administration such as solutions, suspensions, powders, or sustained release formulations. Typically, the compounds are formulated into pharmaceutical compositions using techniques and procedures well known in the art (see e.g., Ansel Introduction to Pharmaceutical Dosage Forms, Fourth Edition, 1985, 126. The formulation should suit the mode of administration.

In one example, pharmaceutical preparation can be in liquid form, for example, solutions, syrups or suspensions. If provided in liquid form, the pharmaceutical preparations can be provided as a concentrated preparation to be diluted to a therapeutically effective concentration before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In another example, pharmaceutical preparations can be presented in lyophilized form for reconstitution with water or other suitable vehicle before use.

Pharmaceutical compositions can include carriers such as a diluent, adjuvant, excipient, or vehicle with which the composition (e.g. GSK3beta inhibitor or recombinant transgene, recombinant viral vector, or recombinant AAV vector) are administered. Acceptable carriers, diluents and adjuvants are nontoxic to recipients and are preferably inert at the dosages and concentrations employed, and may include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

Pharmaceutically acceptable carriers used in parenteral preparations include, for example, aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEENs 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment. Further examples of suitable pharmaceutical carriers are described in “Remington's Pharmaceutical Sciences” by E. W. Martin.

Injectables can be prepared in conventional forms, either as liquid solutions or suspensions, solid forms suitable for solution or suspension in liquid prior to injection, or as emulsions. Preparations for intraprostatic administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, sterile emulsions. The solutions can be either aqueous or nonaqueous.

Titers of recombinant viral vectors to be administered according to the methods of the present disclosure will vary depending, for example, on the particular recombinant viral vector, the mode of administration, the treatment goal, the individual, and the cell type(s) being targeted, and may be determined by methods standard in the art. Titers of recombinant viral vector may range from about 1×10⁶, about 1×10⁷, about 1×10⁸, about 1×10⁹, about 1×10¹⁰, about 1×10¹¹, about 1×10¹², about 1×10¹³, about 1×10¹⁴, or to about 1×10¹⁵ or more DNase resistant particles (DRP) per ml. Dosages may also be expressed in units of viral genomes (vg).

Methods of transducing a target cell with a recombinant viral vector according to the present disclosure, in vivo or in vitro, are contemplated by the present disclosure. The in vivo methods comprise the step of administering an effective dose, or effective multiple doses, of a composition comprising a recombinant viral vector of the present disclosure to an animal (including a human being) in need thereof. If the dose is administered prior to development of a disorder/disease, the administration is prophylactic. If the dose is administered after the development of a disorder/disease, the administration is therapeutic. In embodiments of the present disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the present disclosure is pain and/itch.

Combination therapies are also contemplated by the present disclosure. Combination as used herein includes both simultaneous treatment and sequential treatments. Combinations of methods of the present disclosure with standard medical treatments are specifically contemplated, as are combinations with novel therapies. Combination therapies may include the administration of a recombinant viral vector according to the present disclosure along with a GSK3β inhibitor. Alternatively, a recombinant viral vector and/or GSK3β inhibitor according to the present disclosure may be administered with another therapeutic compound such as, but not limited to, anti-analgesics (e.g., NSAIDS, corticosteroids, acetaminophen, narcotic analgesics (e.g., opioids), muscle relaxants, anti-anxiety drugs, antidepressants, anticonvulsants, and the like) and/or anti-pruritics, such as corticosteroids (e.g., hydrocortisone), counterirritants (e.g., mint oil, menthol, camphor), antihistamines, local anesthetics (e.g., lidocaine, pramoxine, benzocaine) and the like and/or GSK3 inhibitors as provided herein.). In some embodiments, a composition comprises 1, 2, 3, 4, 5, 6, 7, 8, 9, 10, or more different recombinant viral vectors, e.g., rAAVs, each having one or more different transgenes.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of viral (e.g., AAV) components of the recombinant viral vector (e.g., rAAV, and in particular, the AAV ITRs and capsid protein) of the present disclosure may be chosen and/or matched by those skilled in the art taking into account the disease state being treated and the target cells/tissue(s) that are to express the human-delta-catenin protein.

The present disclosure further provides for local administration and systemic administration of an effective dose of a composition (e.g. recombinant viral vector and/or a GSK3beta inhibitor) and compositions of the present disclosure including combination therapy as provided herein. For example, systemic administration is administration into the circulatory system so that the entire body is affected. Systemic administration includes enteral administration such as absorption through the gastrointestinal tract and parenteral administration through injection, infusion or implantation.

In particular, actual administration of a composition according to the present disclosure may be accomplished by using any physical method that will transport the composition into the target tissue of the subject. Administration according to the present disclosure includes, but is not limited to, injection into the ventricles, cisterna magna, spinal theca, muscle, the bloodstream and/or directly into the brain. Simply resuspending the composition in phosphate buffered saline (PBS) has been demonstrated to be sufficient to provide a vehicle useful for muscle tissue expression, and there are no known restrictions on the carriers or other components that can be co-administered with the composition. In cases where the composition comprises a recombinant viral vector (e.g., rAAV), compositions that degrade DNA should be avoided in the normal manner with rAAV. In those cases where the recombinant viral vector comprises rAAV, the capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the subject by transdermal transport. Numerous formulations for both intramuscular injection and transdermal transport have been previously developed and can be used in the practice of the invention. The recombinant viral vector can be used with any pharmaceutically acceptable carrier and/or excipient for ease of administration and handling.

In those embodiments where the composition comprises a recombinant viral vector (including a recombinant AAV vector), the dose to be administered in methods disclosed herein will vary depending, for example, on the particular recombinant viral vector, the mode of administration, the treatment goal, the individual, and the cell type(s) being targeted, and may be determined by methods standard in the art. Titers of each recombinant viral vector (e.g., rAAV) administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1×10¹⁵ vg/kg respectively). Methods for determining titer of viral vectors such as AAV are described in Clark et al., Hum. Gene Ther., 10: 1031-1039 (1999).

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of a composition according to the present disclosure as a free acid (DNA contains acidic phosphate groups) or a pharmacologically acceptable salt can be prepared in water suitably mixed with a surfactant such as hydroxpropylcellulose. A dispersion of composition according to the present disclosure can also be prepared in glycerol, liquid polyethylene glycols and mixtures thereof and in oils. Under ordinary conditions of storage and use, these preparations may contain one or more preservatives of chemical stabilizers to prevent the growth of microorganisms and prolong product life. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. Sterile aqueous media is typically employed.

In some embodiments, recombinant viral vector, e.g., rAAV, compositions are formulated to reduce aggregation of AAV particles in the composition, particularly where high rAAV concentrations are present (e.g., ^{˜}10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference).

rAAV compositions disclosed herein may also be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy use in a syringe is possible. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some embodiments, sterile injectable solutions are prepared by incorporating the composition(s) according to the present disclosure in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Transduction with a recombinant viral vector may also be carried out in vitro. In one embodiment, desired target cells are removed from the subject, transduced with recombinant viral vector and reintroduced into the subject. Alternatively, syngeneic or xenogeneic target cells can be used where those cells will not generate an inappropriate immune response in the subject.

Suitable methods for the transduction and reintroduction of transduced cells into a subject are known in the art. In one embodiment, cells can be transduced in vitro by combining the recombinant viral vector with target cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with recombinant viral vector(s) of the present disclosure can result in sustained expression of human-delta-catenin. The present disclosure thus provides methods of administering/delivering a recombinant viral vector which expresses human-delta-catenin to a subject, preferably a human being. These methods include transducing tissues (including, but not limited to, tissues such as muscle, organs such as liver and brain, and glands such as salivary glands) with one or more recombinant viral vector of the present disclosure. Transduction may be carried out with gene cassettes comprising tissue specific control elements as described herein. The term “transduction” is used to refer to the administration/delivery of the human-delta-catenin gene to a recipient cell either in vivo or in vitro, via a replication-deficient recombinant viral vector of the present disclosure thereby resulting in expression of human-delta-catenin by the recipient cell. Thus, the present disclosure provides methods of administering an effective dose (or doses, administered essentially simultaneously or doses given at intervals) of a recombinant viral vector that encodes human-delta-catenin to a subject in need thereof.

Targeting CNS Tissue

Methods for delivering a transgene to central nervous system (CNS) tissue in a subject are provided herein. A method for delivering a transgene to CNS tissue in a subject may comprise administering a rAAV by a single route or by multiple routes. For example, delivering a transgene to CNS tissue in a subject may comprise administering to the subject, by intravenous administration, an effective amount of a rAAV that crosses the blood-brain-barrier. Delivering a transgene to CNS tissue in a subject may comprise administering to the subject an effective amount of a rAAV by intrathecal administration or intracerebral administration, e.g., by intraventricular injection. A method for delivering a transgene to CNS tissue in a subject may comprise co-administering of an effective amount of a rAAV by two different administration routes, e.g., by intrathecal administration and by intracerebral to administration. Co-administration may be performed at approximately the same time, or different times.

The CNS tissue to be targeted may be selected from cortex, hippocampus, thalamus, hypothalamus, cerebellum, brain stem, cervical spinal cord, thoracic spinal cord, and lumbar spinal cord, for example. The administration route for targeting CNS tissue typically depends on the AAV serotype. For example, in certain instances where the AAV serotype is selected from AAV1, AAV6, AAV6.2, AAV7, AAV8, AAV9, rh.10, rh.39, rh.43 and CSp3, the administration route may be intravascular injection. In some instances, for example where the AAV serotype is selected from AAV1, AAV2, AAV5, AAV6, AAV6.2, AAV7, AAV8, AAV9, rh.10, rh.39, rh.43 and CSp3, the administration route may be intrathecal and/or intracerebral injection. The disclosure describes the use of one or more serotypes, including AAV1, AAV2, AAV4, AAV5, AAV6, AAV7, AAVrh74, AAV8, AAV9, AAV10, AAV 11, AAV12, rh.10, rh.39, rh.43 and CSp3 or AAV13

Intravascular Administration

As used herein the term “intravascular administration” refers to the administration of an agent, e.g., a composition comprising a recombinant viral vector, e.g., rAAV, into the vasculature of a subject, including the venous and arterial circulatory systems of the subject. rAAVs that cross the blood-brain-barrier may be delivered by intravascular administration for targeting CNS tissue. In some cases, intravascular (e.g., intravenous) administration facilitates the use of larger volumes than other forms of administration (e.g., intrathecal, intracerebral). Thus, large doses of rAAVs (e.g., up to 10¹⁵ GC/subject) can be delivered at one time by intravascular (e.g., intravenous) administration. Methods for intravascular administration are well known in the art and include for example, use of a hypodermic needle, peripheral cannula, central venous line, etc.

Intrathecal and/or Intracerebral Administration

As used herein the term “intrathecal administration” refers to the administration of an agent, e.g., a composition comprising a rAAV, into the spinal canal. For example, intrathecal administration may comprise injection in the cervical region of the spinal canal, in the thoracic region of the spinal canal, or in the lumbar region of the spinal canal. Typically, intrathecal administration is performed by injecting an agent, e.g., a composition comprising a rAAV, into the subarachnoid cavity (subarachnoid space) of the spinal canal, which is the region between the arachnoid membrane and pia mater of the spinal canal. The subarchnoid to space is occupied by spongy tissue consisting of trabecula (delicate connective tissue filaments that extend from the arachnoid mater and blend into the pia mater) and intercommunicating channels in which the cerebrospinal fluid is contained. In some embodiments, intrathecal administration is not administration into the spinal vasculature.

As used herein, the term “intracerebral administration” refers to administration of an agent into and/or around the brain. Intracerebral administration includes, but is not limited to, administration of an agent into the cerebrum, medulla, pons, cerebellum, intracranial cavity, and meninges surrounding the brain. Intracerebral administration may include administration into the dura mater, arachnoid mater, and pia mater of the brain. Intracerebral administration may include, in some embodiments, administration of an agent into the cerebrospinal fluid (CSF) of the subarachnoid space surrounding the brain. Intracerebral administration may include, in some embodiments, administration of an agent into ventricles of the brain, e.g., the right lateral ventricle, the left lateral ventricle, the third ventricle, the fourth ventricle. In some embodiments, intracerebral administration is not administration into the brain vasculature.

Intracerebral administration may involve direct injection into and/or around the brain. In some embodiments, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some embodiments, a microinjection pump is used to deliver a composition comprising a recombinant viral vector, e.g., rAAV. In some embodiments, the infusion rate of the composition is in a range of 1 μl/minute to 100 μl/minute. As will be appreciated by the skilled artisan, infusion rates will depend on a variety of factors, including, for example, species of the subject, age of the subject, weight/size of the subject, serotype of the AAV, dosage required, intracerebral region targeted, etc. Thus, other infusion rates may be deemed by a skilled artisan to be appropriate in certain circumstances.

The disclosure also provides that delivery vehicles, such as liposomes, nanocapsules, microparticles, microspheres, lipid particles, vesicles, and the like, may be used for the introduction of the compositions of the present invention into suitable host cells, e.g., neurons. The recombinant viral vector-delivered (e.g., rAAV-delivered) transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Angstrom, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (ie., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

Methods of Use

Another aspect of the disclosure provides methods of treating pain and/or itch in a subject in need thereof, the method comprising, consisting of, or consisting essentially of administering to the subject a therapeutically effective amount of a transgene, nucleic acid cassette, a recombinant virus genetically modified to express human-delta-catenin protein, or a variant thereof, and any pharmaceutical compositions thereof such that the pain and/or itch is treated in the subject.

In some embodiments, the method further comprises administering to the subject a therapeutically effective amount of at least one additional compound. In some embodiments, the compound is selected from the group consisting of a GSK3 inhibitor, anti-analgesics (e.g., NSAIDS, corticosteroids, acetaminophen, narcotic analgesics (e.g., opioids), muscle relaxants, anti-anxiety drugs, antidepressants, anticonvulsants, and the like) and/or anti-pruritics, such as corticosteroids (e.g., hydrocortisone), counterirritants (e.g., mint oil, menthol, camphor), antihistamines, local anesthetics (e.g., lidocaine, pramoxine, benzocaine) and the like.

In another aspect, the disclosure provides methods related to the transgenes, expression cassettes, recombinant viral vectors, recombinant AAV vectors, compositions and pharmaceutical compositions as described in detail above and herein, for example, in the Compositions, Recombinant Viral Vectors and Pharmaceutical Composition sections (collectively “composition” or “compositions”).

In some embodiments, methods of treating pain in a subject in need thereof are provided, including administering a therapeutically effective amount of a composition as described above and herein such that the pain is treated in the subject. In some embodiments, the pain is neuropathic pain or pathological pain. In some embodiments, methods of treating itch in a subject are provided, including administering to the subject a therapeutically effective amount of a composition described above and herein such that the itch is treated in the subject. In some embodiments, the itch is neuropathic or pathological itch.

In some embodiments, methods of increasing KCC2 mRNA levels in a subject are provided, including administering to the subject a therapeutically effective amount of a composition as described above and herein so that the KCC2 mRNA levels are increased in the subject compared to a pre-treatment baseline. In some embodiments, KCC2 mRNA levels are increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline, and in some embodiments, KCC2mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

In some embodiments, methods of reducing intracellular chloride ion levels ([Cl-]i) in a central nervous system cell in a subject are provided, including administering to the subject a therapeutically effective amount of a composition as described above and herein so that the [Cl—]i in a central nervous system cell is reduced in the subject compared to a pre-treatment baseline. In some embodiments, [Cl—]i levels are reduced at least about 10%, or at least about 20%, or at least about 30%, or at least about 40%, or at least about 50%, or at least about 60%, or at least about 70%, or at least about 80%, or at least about 90% compared to pre-treatment baseline.

In some embodiments, methods of increasing chloride ion efflux in a central nervous system cell in a subject are provided, including administering to the subject a therapeutically effective amount of a composition as described above and herein so that the chloride ion efflux is increased in the subject. In some embodiments, chloride ion efflux levels are increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline.

In some embodiments, methods of increasing synaptophysin expression in a central nervous system cell in a subject are provided, including administering to the subject a therapeutically effective amount of a composition as described above and herein so that the synaptophysin expression is increased in the subject as compared to a pre-treatment baseline. In some embodiments, synaptophysin expression is increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline, and in some embodiments, synaptophysin expression is increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

In some embodiments, methods of increasing KCC2 expression in a subject are provided, including administering to the subject a therapeutically effective amount of a composition as described above and herein so that KCC2 expression is increased in the subject compared to pre-treatment baseline. In some embodiments, KCC2 expression is increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline, and in some embodiments KCC2mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

In some embodiments of the methods described above and herein, the methods further include administering to the subject a therapeutically effective amount of at least one additional compound, which compound, in some embodiments, includes a GSK3β inhibitor, an anti-analgesic, a muscle relaxant, an anti-anxiety drug, an antidepressant, an anticonvulsant, and combinations thereof. In some embodiments, the at least one additional compound includes a GSK3β inhibitor a corticosteroid, a counterirritant, an antihistamine, and a local anesthetic and combinations thereof.

In some embodiments, the GSK3β inhibitor is selected from the group consisting of CHIR-99021 (CT99021) HCl, SB-216763, CHIR-98014, TWS119, Tideglusib, SB-415286, BIO (6-bromoindirubin-3′-oxime), kenpaullone, CHIR-99021 (CT99021), AZD2858, AZD1080, AR-A014418, TDZD-8, LY2090314, IM-12, BIO-acetoxime, Indirubin, 5-Bromoindole, 2-D08, Bilinin, 1-Azakenpaullone, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium and any combinations thereof. In some embodiments, the GSK3β inhibitor is selected from the group consisting of kenpaullone (9-bromo-7,12-dihydro-indolo [3,2-d] [1]benzazepin-6(5H)-one), NSC180515 (2-Acetyl-2,3,4,5-tetrahydrooxonine-6,9-dione), NSC79456 (n-(2,4-Dimethylphenyl)-2-hydroxy-3-nitrobenzamide), or NSC33006 (N-[4-(1,3-benzothiazol-2-yl)phenyl]acetamide), and in some embodiments, the GSK3β inhibitor is kenpaullone (9-bromo-7,12-dihydro-indolo [3,2-d][1]benzazepin-6(5H)-one).

Another aspect of the disclosure is based on the findings by the inventor that renormalizing inhibitory neurotransmission can be achieved by GSK3β inhibition.

Accordingly, another aspect of the present disclosure provides a method of treating pain and/or itch in a subject in need thereof, the method comprising, consisting of, or consisting essentially of administering to the subject a therapeutically effective amount of a GSK3β inhibitor, or a pharmaceutical composition comprising a GSK3β inhibitor and a pharmaceutically acceptable carrier and/or excipient, such that the pain and/or itch is treated in the subject.

As used herein, the term “GSK3β inhibitor” refers to any compound/molecule (e.g., antibodies, small molecules, oligonucleotides, siRNAs, miRNAs, etc.) that is able to inhibit or reduce the function of the GSK3β protein, or inhibit or reduce the expression of the GSK3 gene. Suitable inhibitors may include, but are not limited to, CHIR-99021 (CT99021) HCl, SB-216763, CHIR-98014, TWS119, Tideglusib, SB-415286, BIO (6-bromoindirubin-3′-oxime), kenpaullone, CHIR-99021 (CT99021), AZD2858, AZD1080, AR-A014418, TDZD-8, LY2090314, IM-12, BIO-acetoxime, Indirubin, 5-Bromoindole, 2-D08, Bilinin, 1-Azakenpaullone, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium and any pharmaceutical compositions thereof.

In some embodiments, the disclosure provides methods of treating pain and/or treating itch in a subject in need thereof, including administering a therapeutically effective amount of a GSK3β inhibitor, or a pharmaceutical composition including a GSK3β inhibitor and a pharmaceutically acceptable carrier and/or excipient, such that the pain is treated in the subject. In some embodiments, the pain and/or itch is neuropathic or pathological in nature.

In some embodiments, methods of increasing KCC2 mRNA levels in a subject are provided, including administering to the subject a therapeutically effective amount of a GSK3β inhibitor, or a pharmaceutical composition including a GSK3β inhibitor and a pharmaceutically acceptable carrier and/or excipient, so that the KCC2 mRNA levels are increased in the subject. In some embodiments, KCC2 mRNA levels are increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline, and in some embodiments, KCC2mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

In some embodiments, methods of reducing intracellular chloride ion levels ([Cl-]i) in a central nervous system cell in a subject are provided, including administering to the subject a therapeutically effective amount of a GSK3β inhibitor, or a pharmaceutical composition including a GSK3β inhibitor and a pharmaceutically acceptable carrier and/or excipient, so that the [Cl—]i in a central nervous system cell is reduced in the subject. In some embodiments, [Cl—]i levels are reduced at least about 10%, or at least about 20%, or at least about 30%, or at least about 40%, or at least about 50%, or at least about 60%, or at least about 70%, or at least about 80%, or at least about 90% compared to pre-treatment baseline.

In some embodiments, methods of increasing chloride ion efflux in a central nervous system cell in a subject are provided, including administering to the subject a therapeutically effective amount of a GSK3β inhibitor, or a pharmaceutical composition including a GSK3β inhibitor and a pharmaceutically acceptable carrier and/or excipient, so that the chloride ion efflux is increased in the subject. In some embodiments, chloride ion efflux levels are increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline.

In some embodiments, methods of increasing synaptophysin expression in a central nervous system cell in a subject in need thereof, comprising administering to the subject a therapeutically effective amount of a GSK3β inhibitor, or a pharmaceutical composition comprising a GSK3β inhibitor and a pharmaceutically acceptable carrier and/or excipient, such that the synaptophysin expression is increased in the subject. In some embodiments, synaptophysin expression is increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline, and in some embodiments, synaptophysin expression is increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

In some embodiments, methods of increasing KCC2 expression in a subject are provided, including administering to the subject a therapeutically effective amount of a GSK3β inhibitor, or a pharmaceutical composition including a GSK3β inhibitor and a pharmaceutically acceptable carrier and/or excipient, so that KCC2 expression is increased in the subject. In some embodiments, KCC2 expression is increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline, and in some embodiments KCC2mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

In some embodiments of the methods described above, the methods further include administering to the subject a therapeutically effective amount of at least one additional compound, which compound, in some embodiments, includes compositions including a transgene, an expression cassette, or a recombinant viral vector as described above and herein (each, in some embodiments, pharmaceutical compositions that include a pharmaceutically acceptable carrier and/or excipient), an anti-analgesic (e.g., NSAIDS, corticosteroids, acetaminophen), narcotic analgesics (e.g., opioids), a muscle relaxant, an anti-anxiety drug, an antidepressant, an anticonvulsant, and combinations thereof. In some embodiments, the additional compound can include compositions including a transgene, an expression cassette, or a recombinant viral vector as described above and herein (each, in some embodiments, pharmaceutical compositions that include a pharmaceutically acceptable carrier and/or excipient), and/or anti-pruritics, such as a corticosteroid (e.g., hydrocortisone), a counterirritant (e.g., mint oil, menthol, camphor), an antihistamine, and a local anesthetic (e.g., lidocaine, pramoxine, benzocaine) and combinations thereof.

In the methods described in all relevant aspects and embodiments of the disclosure, the central nervous system cell is a neuron.

In the methods described in all relevant aspects and embodiments of the disclosure, the composition can be administered by any administration route, including intrathecally, intra-cerebroventricularly, intra-cerebrally, perispinally, intra-spinally, intravenously and others.

In embodiments of this aspect related to methods of treating pain and/or treating itch in a subject in need thereof, including administering a therapeutically effective amount of a GSK3β inhibitor, or a pharmaceutical composition including a GSK3β inhibitor, the GSK3β inhibitor includes one or more of CHIR-99021 (CT99021) HCl, SB-216763, CHIR-98014, TWS119, Tideglusib, SB-415286, BIO (6-bromoindirubin-3′-oxime), kenpaullone, CHIR-99021 (CT99021), AZD2858, AZD1080, AR-A014418, TDZD-8, LY2090314, IM-12, BIO-acetoxime, Indirubin, 5-Bromoindole, 2-D08, Bilinin, 1-Azakenpaullone, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium and any combinations thereof.

In some embodiments, the GSK3β inhibitor includes one or more of kenpaullone (9-bromo-7,12-dihydro-indolo [3,2-d] [1]benzazepin-6(5H)-one), NSC180515 (2-Acetyl-2,3,4,5-tetrahydrooxonine-6,9-dione), NSC79456 (n-(2,4-Dimethylphenyl)-2-hydroxy-3-nitrobenzamide), or NSC33006 (N-[4-(1,3-benzothiazol-2-yl)phenyl]acetamide), and in some embodiments, the GSK3β inhibitor is kenpaullone (9-bromo-7,12-dihydro-indolo [3,2-d][1]benzazepin-6(5H)-one).

In some embodiments, the methods further comprising administering to the subject a therapeutically effective amount of at least one additional compound, which, in some embodiments, includes one or more compositions (e.g., transgenes, expression cassettes, recombinant viral vectors, recombinant AAV vectors, compositions and pharmaceutical compositions as described in detail above and herein, for example, in the Compositions, Recombinant Viral Vectors and Pharmaceutical Composition sections), an anti-analgesic, a muscle relaxant, an anti-anxiety drug, an antidepressant, an anticonvulsant, and combinations thereof. In some embodiments, the additional compound includes one or more compositions (as above), a corticosteroid, a counterirritant, an antihistamine, and a local anesthetic and combinations thereof.

In some embodiments, the central nervous system cell is selected from the group consisting of neurons, oligodendrocytes, astrocytes, brain parenchyma cells, and Purkinje cells, and in some embodiments, the central nervous system cell is a neuron.

In some embodiments of all aspects of the disclosure, compositions (in its broadest sense, e.g., one or more of transgenes, expression cassettes, recombinant viral vectors, recombinant AAV vectors, compositions and pharmaceutical compositions as described in detail above and herein, for example, in the Compositions, Recombinant Viral Vectors and Pharmaceutical Composition sections, GSK3β inhibitors, anti-analgesic, a muscle relaxant, an anti-anxiety drug, an antidepressant, an anticonvulsant, corticosteroid, a counterirritant, an antihistamine, and a local anesthetic as described herein) are administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually.

In some embodiments, the composition is administered intrathecally, intra-cerebroventricularly, intra-cerebrally, perispinally, intra-spinally, and in some embodiments, the composition is administered intrathecally.

In an aspect, the disclosure provides a method for delivering a transgene to central nervous system tissue in a subject, the method comprising administering an effective amount of a rAAV comprising a promoter operably linked with a transgene to central nervous system (CNS) tissue by intrathecal administration, wherein the rAAV infects cells of the CNS of the subject, wherein the transgene encodes a polypeptide of any one of SEQ ID NO:21, 23, 25, 27, 29, 31 or 33 (or a sequence having at least 50%, 60%, 65%, 70%, 75%, 80%, 85%, 90%, 95%, 96%, 97%, 98% or 99% sequence identity to the sequence set forth in SEQ ID NO: 21, 23, 25, 27, 29, 31 or 33), wherein the polypeptide reduces pathologic pain or itch.

In some embodiments, the intrathecal administration is in the lumbar region of the subject; in some embodiments the intrathecal administration is in the cervical region of the subject, and in some embodiments the intrathecal administration is in the thoracic region of the subject.

In some embodiments, the cells of central nervous system tissue include oligodendrocytes, astrocytes, neurons, brain parenchyma cells, and/or Purkinje cells, and in some embodiments, the cells of central nervous system tissue are neurons.

As described in the Examples herein, an unbiased screen was conducted of cell growth-regulating compounds with the insight that a sizable fraction of them function by interfering with epigenetic and transcriptional machinery. Since mature neurons do not divide, these compounds were attractive candidates to upregulate gene expression of KCC2 and thereby lower neuronal chloride levels. The inventor identified such compounds by rigorous iterations of primary and secondary screening. In particular, the inventor identified kenpaullone (KP), a glycogen synthase kinase-3 (GSK3)/cyclin-dependent kinase (CDK) inhibitor (Schultz et al., 1999, J Med Chem 42:2909-2919; Zaharevitz et al., 1999, Cancer research 59:2566-2569). KP functions as an analgesic and antipruritic in preclinical mouse models. The data described in the Examples herein suggest that a cellular mechanism of action of KP in neurons is based on its GSK3ß-inhibitory function which causes increased KCC2 gene expression, which in turn relies on nuclear transfer of the neuronal catenin, δ-catenin (referred to herein as δ-cat, Δ-cat, or delta-cat) (Arikkath et al., 2009, J Neurosci 29:5435-5442; Turner et al., 2015, Nature 520:51-56). In the nucleus, the inventor found that δ-cat enhances KCC2 gene expression via Kaiso transcription factors (Rodova et al., 2004, Mol Cell Biol 24:7188-7196). Increased KCC2 gene expression led to increased KCC2 chloride extrusion in neurons. The inventor also documented KCC2 expression enhancement in the spinal cord dorsal horn (SCDH) of mice with nerve injury, including more negative, thus electrically more stable GABA-evoked chloride reversal potential.

The following Examples are provided by way of illustration and not by way of limitation.

### EXAMPLES

Materials and Methods

Screening in Primary Cortical Neurons from KCC2-LUC Transgenic Mice

Transgenic mice that express red-shifted luciferase (LUC) under the control of the KCC2 promoter (−2052/+476, described in (Yeo et al., 2009, J Neurosci 29:14652-14662)), inserted into the Rosa26 locus, were previously described (Liedtke et al., 2013, Small 9:1066-1075; Yeo et al., 2013, Proc Natl Acad Sci USA 110:4315-4320). Primary cortical neuronal cultures were generated from newborn (p0) mice of this line as previously described (Liedtke et al., 2013, Small 9:1066-1075; Yeo et al., 2013, Proc Natl Acad Sci USA 110:4315-4320).

Cytosine arabinoside (2.5 m) was added to cultures on the second day after seeding [2 d in vitro (DIV)] to inhibit the proliferation of non-neuronal cells. Cell suspension was plated at a density of 1×10⁶ cells/ml onto 24-well tissue-culture dishes coated with poly-d-lysine. Cortical neuronal cultures prepared by this method yielded a majority population of neuronal cells, with negligible glia contamination, as evidenced by the absence of GFAP by Western blotting.

After a week in culture, neurons were treated with compounds (100 nM for 48 h). LUC activity was then determined for each compound. Culture supernatant was removed and cells were lysed with 150 μl lysis buffer (Targeting Systems CA, USA cat. CLR1). LUC activity was measured with a RedLuciferase Assay kit (Targeting Systems cat. FLAR) according to the manufacturer's instructions. A Veritas microplate luminometer was used to measure luminescence; for each treatment triplicates of 40 μl cell lysates (from each 24-well tissue-culture dish) were used and 25 μl substrate was injected per well. To evaluate the quality of screening methodology, Z′ factor (Zhang et al., 1999, Journal of biomolecular screening 4:67-73) was ascertained using 0.5% (v/v) DMSO as negative control and Trichostatin A as positive control. Relative Light units (RLU) for each treatment was derived from Light Units (compound treatment)/vehicle treatment (0.5% DMSO).

The primary screen encompassed three levels of LUC measurements. The first level of screening yielded a total of 137 compounds with cut-off RLU>125% LUC activity. These 137 compounds were subject to second round of screening, which was carried out in duplicate independent assays to yield the top 103 compounds sorted for highest RLU. The 103 compounds were then subjected to a third round of screening carried out in duplicate independent assays to yield the top 40 compounds which were again ranked. The best 22 of these 40 compounds (ranked for highest RLU activity) were subjected to secondary screening: Cultured neurons were treated with the 22 compounds; RT-qPCR and Clomeleon imaging methodologies (Yeo et al., 2009, J Neurosci 29:14652-14662) were used to determine effects of compounds on KCC2 mRNA expression and [Cl—]i, respectively. Each compound was ranked based on composite scores from primary and secondary screening.

Compound Libraries

NCI compound libraries Natural Products II and Mechanistic Diversity Set II were obtained from the National Cancer Institute. See also: https://dtp.cancer.gov/organization/dscb/obtaining/available_plates.htm.

Human Neuronal Cultures

The neuron-enriched cultures were established from fetal cortical specimens at 15-20 weeks of gestation. The protocols for tissue processing complied with all federal and institutional guidelines. The cultures were plated on PEI (polyethyleneimine solution) substrate and maintained in Neurobasal media supplemented with B27 as previously described (Pelsman et al., 2003, International journal of developmental neuroscience 21:117-124; Yeo et al., 2013, Proc Natl Acad Sci USA 110:4315-4320).

For RT-qPCR, the cell pellets were collected after treatments with vehicle (0.1% DMSO) or KP (50, 100, and 400 nM) for 48 h starting at day 6 in vitro.

For immunostaining, the cultures were treated for 2-4 days with vehicle or 400 nM KP, fixed with 4% PFA at day 10, and processed for double-staining with anti-KCC2 and anti-synaptophysin or anti-NeuN (see Table 1).

TABLE 1
Primary Antibodies

| Antibody | Source |
|---|---|
| anti-KCC2 | Millipore (07-432) (mouse spinal cord) |
| | ThermoFisher Scientific (PA5-78544) (human, mouse, rat ICC) |
| anti-β_{III} Tubulin | Abeam (mouse, ab78078; rabbit, ab229590) |
| anti-β-catenin | SigmaAldrich (PLA0230) |
| anti-δ-catenin | SigmaAldrich (MABN2254) |
| anti-Synaptophysin | ThermoFisher (MA1-213) |
| anti-FLAG | SigmaAldrich (F3165) |
| anti-NeuN | BioLegend (834501) |

The 0.34 micrometer confocal slices through entire cell layers at different optical fields (n=17-28 for each group) of the fixated cultures were acquired using a Zeiss LSM700 confocal microscope.

Image Z-stacks were analyzed using Imaris 9.2.1 software. Optical density intensity sum values for each stack/channel were divided by corresponding data volumes and the resulting values were normalized to the number of specifically labeled cells within each stack. There were no significant differences in the number of cells between vehicle and KP-treated groups.

δ-Catenin DNA Constructs, Transgenesis Vectors

Plasmid containing human δ-catenin (CTNND2, NM_001288717) open reading frame was obtained from GeneCopeia (EX-A4285-M02) and cloned into pCMV-ENTER vector (Origene PS100001). Site-directed mutagenesis using Phusion DNA Polymerase enzyme (Thermofisher F549L) in conjunction with complementary primers bearing the specific mutation were used to generate the S276A S-catenin mutation S276A. PCR was followed by Dpn1 enzyme digestion to remove parental plasmid DNA. All constructs were verified by sequencing. pCS-CMV-tdTomato plasmid was obtained from Addgene (cat. #30530).

Plasmid pAAV-hSyn-eNpHR 3.0-EYFP from Addgene (26972) was cut with AgeI and HindIII enzymes to excise the eNpHR 3.0-EYFP open reading frames. The control tdTomato open reading frame as well as the wild-type and mutant delta catenin open reading frames were generated with AgeI and HindIII ends by PCR and subsequently inserted into the AgeI/HindIII digested pAAV-hSyn plasmid. Orientation and sequence fidelity in the final constructs were verified by PCR and sequencing. AAV9 particles were packaged by the Duke University Viral Vector Core facility and were used at a titer of 10¹² viral genome copies per mL.

KCC2 Promoter Luciferase Reporter Assays

A fragment of the mouse KCC2 gene promoter (position −2052 kbp to +476 kbp) was amplified from genomic DNA prepared from cultured mouse primary glial cells. A 2.5 kb PCR fragment was cloned into the pGL4.17-Basic Vector (Promega) to generate the KCC2 promoter reporter construct. TCF and Kaiso binding sites were identified in this fragment. Using wild-type construct pGL4.17-KCC2 as a template, site-directed mutagenesis using Phusion DNA Polymerase enzyme (Thermo Fisher F549L) in conjunction with complementary primers bearing the specific mutation were used to mutate the Kaiso and TCF DNA-binding sites. PCR was followed by Dpn1 enzyme digestion to remove parental plasmid DNA. All constructs were verified by sequencing.

N2a cells were grown to 90% confluency in 24-well dishes in 0.4 mL of medium (DMEM, 2% Fetal Bovine Serum, 2 mM glutamine, 1% Non-essential amino acids, and 1% Penicillin/Streptomycin). Cells were transiently transfected using TurboFect reagent (Thermo Fisher R0531), with 500 ng of the pGL4.17-constructs plus 20 ng of the control Renilla plasmid (Promega, E2231) to normalize for transfection efficiency. Twenty-four hours after transfection, luminescence was measured using the Dual-Luciferase® Reporter Assay System (Promega) in a microplate luminometer (Veritas, Turner Biosystems). Mutant promoters were compared to WT, and the response of the respective promoter to KP (400 nM) was measured. Three independent transfection experiments were carried out and LUC assays were done in triplicates for each transfection. RLU is expressed as firefly luciferase activity relative to Renilla LUC activity.

Chemicals

kenpaullone compound was synthesized by the Duke Small Molecule Synthesis Facility to >98% purity, verified by LC/MS. CLP257, ICG-001, TWS119, CHIR99201, and VU0240551 were obtained from Tocris. GW801372X, GW778894X, GW300660X, GW779439X, and GW305178X were supplied by the Structural Genomics Consortium (SGC) at UNC-Chapel Hill.

Animals

C57bl/6J male mice (10-12 weeks old) were obtained from The Jackson Lab (Bar Harbor, Me.). KCC2-LUC mice were generated by the Liedtke Lab at Duke University and continued as a line within our mouse colony. All animal procedures were approved by The Duke University IACUC.

RT-qPCR

Total RNA was isolated from cultured cell samples using Directzol RNA miniprep kit (ZymoResearch). The protocol includes DNAse digestion to exclude genomic DNA from preparations. Total RNA (1 μg) was reverse transcribed using oligo primers (dT) and SuperScriptIII first-strand synthesis kit (Invitrogen). Gene expression was assessed by quantitative real-time PCR using 2×SYBR Green Master Mix (Qiagen) and a three-step cycling protocol (anneal at 60° C./elongate at 72° C., denature at 95° C.). Specificity of primers was verified by dissociation/melting curve for the amplicons when using SYBR Green as a detector. All reactions were performed in triplicates. The amount of target messenger RNA (mRNA) in the experimental group relative to that in the control was determined from the resulting fluorescence and threshold values (Ct) using the ΔΔCt method. β_{III}-tubulin was used as housekeeping gene.

RT-qPCR from Cultured Neuronal Cells and Microdissected Spinal Cord Dorsal Horn

oligodT-initiated reverse transcription of 1 μg total RNA, DNA-se treated, was subjected to RT-qPCR using primers specific for KCC2 for rat and KCC2 for human sequences, normalized for neuronal β_{III}-tubulin, as previously described (Yeo et al., 2009, J Neurosci 29:14652-14662). For cultured neurons, total RNA was extracted from pelleted cells, and for spinal cord tissue it was extracted from microdissected lumbar spinal cord dorsal horn.

Behavioral Assessments

For pain-related behavior, mechanical allodynia was assessed with von Frey filaments (Ugo Basile, Italy). The mice were placed on a 5×5-mm wire-mesh grid floor in individual compartments to avoid visual stimulation and allowed to adapt for 0.5 h prior to the von Frey test. The von Frey filament was then applied to the middle of the plantar surface of the hind paw with 5 g force. The withdrawal responses following the hind paw stimulation were measured at least three times, and the mechanical allodynia, which was defined as an increase in the number of withdrawal responses to the stimulation, was compared.

In addition, paws were stimulated with heat from underneath applied by an infrared beam (Hargreaves' test apparatus, IR level 40; Ugo Basile), and withdrawal latencies were recorded.

To assess scratching behavior as a behavioral correlate of itch, mice were shaved at the dorsal neck where topical application of 0.5% DNFB was applied (day 1). At days 5, 7, 9, and 11, mice received intraperitoneal (i.p.) injection of either KP or vehicle followed by 0.25% DNFB topical applications 4 hr later. On day 12, mice were allowed to acclimate to a Plexiglas chamber for at least 30 mins before performing itch behavior test. Scratching behavior was recorded by a Panasonic video camera for a 30-min observation period. Hind limb scratching behavior directed toward the shaved area at the nape of neck was observed. One scratch is defined as a lifting of the hind limb toward the injection site and then a replacing of the limb back to the floor, regardless of how many scratching strokes take place between those two movements. Behavioral analysis was conducted by observers blinded to treatment procedure. DNFB-induced scratching behavior was recorded on day 12 for 1 h. One scratch bout is defined as a lifting of the hind limb toward the injection site and then a replacing of the limb back to the floor, regardless of how many scratching strokes take place between those two movements. To determine the effect of KP on scratching behavior induced by DNFB, 30 mg/kg of KP was i.p. injected 20 min before each of DNFB challenge from day 5 to day 11.

For assessment in rotarod (RR), all animals received training prior to experiments; mice were placed on the RR apparatus set in an accelerating rotational speed mode (3-30 rpm, 300 s max) per trial. Following training, the average time to fall from the rotating cylinder over three trials was recorded as baseline latency (4-40 rpm, 300 s max/trial). Mice were injected daily with either vehicle or drug compounds before RR tests. Latency to fall was measured (4-40 rpm, 300 s max/trial (inter-trial interval is at least 15 min). The average latency to fall from the rod was recorded for each animal.

Conditioned place preference (CPP) was conducted using a CPP box, which consists of two conditioning chambers distinguished by visual and sensory cues, along with a small buffering chamber. All mice received a 3-day preconditioning habituation period with free access to both conditioning chambers and the time spent in each chamber was recorded for 15 min on day 3 after habituation. On conditioning days (day 4-10), mice first received the vehicle control (i.p. 5% DMSO, 5% Tween-80 in normal saline) paired with a randomly chosen chamber in the morning. After 4 hours, mice received KP (i.p. 30 mg/kg) or vehicle, paired with the other chamber. During the conditioning, mice were allowed to stay only in the paired chamber for 15 min without access to other chambers. On test day (d11), mice were placed in the buffering chamber with free access to both conditioning chambers and choice behavior was recorded for 15 min. The CPP scores were calculated as post-conditioning time minus preconditioning time spent in the paired chamber.

Chromatin Immunoprecipitation

ChIP assay was carried out as described previously (Yeo et al., 2009, J Neurosci 29:14652-14662; Yeo et al., 2013, Proc Natl Acad Sci USA 110:4315-4320). Primary cortical neurons (0.7×10⁶) were used for each ChIP experiment. Cells were crosslinked with 1% formaldehyde for 30 min, washed twice with cold PBS, resuspended in lysis buffer [1% SDS, 10 mm EDTA, and 50 mm Tris-HCl, pH 8.0, with protease inhibitor cocktail (Roche)], and sonicated for 15 s pulses. The lysates were clarified by centrifugation at 10,000 rpm for 10 min at 4° C. in a microcentrifuge. One-tenth of the total lysate was used as input control of genomic DNA. Supernatants were collected and diluted in buffer (1% Triton X-100, 2 mm EDTA, 150 mm NaCl, 20 mm Tris-HCl, pH 8.0, and protease inhibitor cocktail) followed by immunoclearing with 1 mg of salmon sperm DNA, 10 ml of rabbit IgG, and 20 ml of protein A/G-Sepharose (Santa Cruz Biotechnology) for 1 h at 4° C. Immunoprecipitation was performed overnight at 4° C. with 2 mg of each specific antibody. Precipitates were washed sequentially for 10 min each in TSE1 buffer (0.1% SDS, 1% Triton X-100, 2 mm EDTA, 150 mm NaCl, and 20 mm Tris-HCl, pH 8.0), TSE2 (TSE1 with 500 mm NaCl), and TSE3 (0.25 m LiCl, 1% NP-40, 1% deoxycholate, 1 mm EDTA, and 10 mm Tris-HCl, pH 8.0). Precipitates were then washed twice with 10 mm Tris/0.1 mm EDTA, pH 7.8 and extracted with 1% SDS containing 0.1 m NaHCO₃. Eluates were pooled and heated at 65° C. for 4 h to reverse formaldehyde crosslinking. DNA fragments were purified with Qiagen Qiaquick spin kit. For ChIP PCR, 1 l of a 25 l DNA extraction was used.

Immuno-Cytochemistry of Cultured Neurons

Immunocytochemistry labeling of cultured neuronal cells was carried out as previously described (Liedtke et al., 2013, Small 9, 1066-1075; Yeo et al., 2009, J Neurosci 29:14652-14662; Yeo et al., 2013, Proc Natl Acad Sci USA 110:4315-4320). Primary antibodies are shown in the antibody table. Anti-KCC2 primary antibodies were validated with developing rat primary cortical neurons; we observed an increase in staining pattern that tightly matched increase of KCC2 mRNA expression (Liedtke et al., 2013, Small 9:1066-1075; Yeo et al., 2009, J Neurosci 29:14652-14662.; Yeo et al., 2013, Proc Natl Acad Sci USA 110:4315-4320). Secondary antibodies used were goat anti-mouse IgG Alexa Fluor 594 (Invitrogen A11032) and goat anti-rabbit IgG Alexa Fluor 594 (Invitrogen A11012). DAPI stain was obtained from Sigma Aldrich (D9542). Stained cells were observed using an inverted confocal microscope (Zeiss LSM780).

We obtained stacks of images recorded at 0.35 μm intervals through separate channels with a 63× oil-immersion lens (NA, 1.40, refraction index, 1.45). Zen software (Zeiss) was used to construct composite images from each optical series by combining the images recorded through the different channels, and the same software was used to obtain Z projection images (image resolution: 1024×1024 pixels; pixel size: 0.11 μm). ImageJ was used for morphometry.

Spinal Cord Immuno-Histochemistry

All mice were deeply anaesthetized with isoflurane and then transcardially perfused with ice-cold 4% paraformaldehyde in 0.1 M phosphate buffer, pH 7.4 (4% PFA). Dissected spinal cord samples were then post-fixed overnight in 4% PFA at 4° C., cryoprotected in a 20% sucrose solution in PBS at 4° C., frozen in Tissue-Tek OCT (Sakura), and stored at −80° C. until sectioning. Samples were sectioned at 20 μm using a cryostat (Microm HM 505N). The sections were blocked with 2% bovine serum albumin (BSA) in PBS with 0.3% Triton X-100 (Blocking solution) at room temperature for 1 h. The sections were treated with primary antibody in blocking solution at 4° C. overnight. The sections were washed three times followed by secondary antibody treatment at 4° C. for 2 hours. Anti-KCC2 antibody was validated as described above for immuno-cytochemistry. The goat anti-rabbit IgG Alexa Fluor 488 was obtained from Invitrogen (A-11008). Morphometry was conducted using ImageJ with region-of-interest Rexed laminae I-II.

Chloride Imaging

Chloride imaging of primary cultured cortical neurons was conducted as previously described (Kuner and Augustine, 2000, Neuron 27:447-459; Liedtke et al., 2013, Small 9:1066-1075; Yeo et al., 2009, J Neurosci 29:14652-14662). A Clomeleon expression plasmid was transfected into primary cortical neurons by electroporation (Amaxa Nucleofector Device). Transfected neurons were verified by yellow fluorescent protein (YFP) fluorescence, and ratiometric images (excitation at λ=434 nm, dual emission at λ=485 and 535 nm; for resting chloride, six stable frames at a rate 12 of per minute were captured, which were averaged) were acquired using RATIOTOOL program. Calibration of Clomeleon signals (535 nm/485 nm emission ratio) was performed by using tributyltin-nigericin to establish a standard curve, which was then normalized for measured intraneuronal pH to take into account the pH sensitivity of Clomeleon.

Spinal Cord Dorsal Horn Electrophysiology

For spinal cord slice preparation, adult (5-7 weeks) male mice were anesthetized with urethane (1.5-2.0 g/kg, i.p.). The lumbosacral spinal cord was microsurgically removed and submerged into ice-cold dissection media which was saturated with 95% O₂ and 5% CO₂ at room temperature. After extraction and still under anesthesia, animals were euthanized. Transverse slices (300-400 m) were cut using a vibrating microslicer (VT1200s Leica). The slices were incubated at 32° C. for at least 30 min in regular artificial cerebrospinal fluid (aCSF), equilibrated with 95% O₂ and 5% CO₂.

The following solutions were used: Dissection solution: Sucrose 240 mM, NaHCO₃ 25 mM, KCl 2.5 mM, NaH₂PO₄ 1.25 mM, CaCl₂ 0.5 mM, MgCl₂ 3.5 mM (Cheng et al., 2017, Nature neuroscience 20, 804-814). Regular artificial cerebrospinal fluid (ACSF): NaCl 117 mM, KCl 3.6 mM, MgCl₂ 1.2 mM, CaCl₂ 2.5 mM, NaHCO₃ 25 mM, NaH₂PO₄ 1.2 mM, glucose 11 mM. The pH value of ACSF or dissection solution was adjusted to 7.4 when saturated with the gas. Normal intrapipette solution (pH 7.2 and 310 mOsm): K-methylsulfate 115 mM, KCl 25 mM, MgCl₂ 2 mM, HEPES 10 mM, GTP-Na 0.4 mM and Mg-ATP 5 mM.

Electrophysiological recordings were conducted as follows. A slice was placed in the recording chamber and completely submerged and superfused at a rate of 2-4 ml/min with aCSF saturated with 95% O₂ and 5% CO₂ at room temperature. Perforated patch-clamp was used to avoid alteration of the [Cl—]i. To measure the chloride equilibrium potential (E_{C1}), gramicidin D (80 μg/mL with 0.8% DMSO final concentration, from an 8 mg/mL stock in DMSO) was added to the intrapipette solution, and 6-cyano-7-nitroquinoxaline-2,3-dione (CNQX, 10 μM), D_{L}-2-amino-5-phosphonovaleric acid (APV, 50 μM), and tetrodotoxin (TTX, 0.5 μM) were added to the aCSF solution. The tip of the patch pipette was filled with the normal intrapipette solution while the rest of the pipette contained the gramicidin-containing solution. After forming a seal on the membrane, we waited ˜30 min for the gramicidin to induce sufficient cation-selective pores in the membrane and lowered the series resistance to below 100 MΩ. Membrane potential measurements were corrected for liquid junction potential, which was measured as in (Jiang et al., 2014, Journal of neurophysiology 111:991-1007). GABA (1 mM) was puffed locally and instantaneously, and the puff pipette was aimed toward the recording pipette. To determine the reversal potential of GABA-evoked currents, voltage ramps were applied from +8 to −92 mV over 200 ms at a holding potential of −42 mV. Since the voltage ramp might elicit a basal current, a control voltage ramp was applied, and 1 min later GABA was puffed followed by another voltage ramp (Billups and Attwell, 2002, The Journal of physiology 545:183-198). The reversal potential was analyzed as previously described (Billups and Attwell, 2002, The Journal of physiology 545:183-198).

Signals were acquired using an Axopatch 700B amplifier and analyzed with pCLAMP 10.3 software. Only neurons with resting membrane potential <−50 mV and stable access resistance were included.

Drug Affinity Responsive Target Stability (DARTS) assay

Cultured primary rat cortical neurons were treated with either vehicle DMSO (0.1%) or 20 μM KP for 30 h. Cells were lysed in ice-cold lysis buffer (Tris.Cl pH8 50 mM, NaCl 150 mM, NP40 0.5%, N-dodecyl-b-D-maltoside 0.5%, Phosphatase Inhibitor (Pierce #88667) and Protease Inhibitor (Roche #11836153001)). Protein concentrations were determined by Bio-Rad DC Protein Assay kit using bovine albumin as standard. All steps were performed on ice. Samples were warmed to room temperature and digested with pronase (final concentration 1:500) for 30 min at 30° C. Digestion was halted using 0.5M EDTA. Only proteins not bound to KP were digested. The protein mixture was dialyzed using dialysis cassettes (Thermo Fisher 66203, 2K MWCO) and analyzed by LC-MS/MS method to identify proteins that are bound to KP, the latter step carried out in the Duke Proteomics Core Laboratory.

Kinome Analysis

Cultured rat primary cortical neurons were treated with either vehicle DMSO (0.1%) or 1 μM KP for 1 h/24 h. Cells were lysed in non-detergent-containing buffer (Tris.Cl pH8 50 mM, NaCl 150 mM, 0.5% Phosphatase Inhibitor (Pierce #88667) and Protease Inhibitor (Roche #11836153001)). 500 μL was removed and solid urea was added to a final concentration of 8M. Samples were sonicated for further solubilization. After clearing of insoluble material by centrifugation, protein concentration was measured by Bradford assay. 250 μg of total protein was removed from each sample and solubilization buffer was added to normalize all samples to 0.93 μg/μL protein. Samples were then spiked with bovine alpha-casein to 30 fmol/μg of total protein. Samples were reduced with 10 mM DTT at 32° C. for 45 min and then alkylated with 20 mM iodoacetamide at room temperature for 30 min. Samples were trypsin digested at 1:25 (enzyme-to-protein) overnight at 32° C. Following acidification with TFA to pH 2.5, samples were subjected to a C18 solid-phase extraction cleanup. Eluted peptides were split 80% for phosphopeptide analysis and 20% reserved for unbiased differential expression. The phosphopeptide fraction (200 μg) was then frozen and lyophilized prior to phosphopeptide enrichment.

TiO2 Enrichment. Samples were resuspended in 65 μL of 1M glycolic acid in 80% MeCN/1% TFA and were enriched on TiO2 resin using a 10 μL GL Sciences microliter TiO2 spin tips following an established protocol (www.genome.duke.edu/cores/proteomics/samplepreparation/documents/GL_SpinColumnPr otocol_bmr_ejs_mt_061713.pdf). After elution and acidification, samples were lyophilized to dryness and resuspended in 100 μL of 0.15% TFA in water. After cleanup using a C18 STAGE tip, and resuspension in 2% acetonitrile, 0.1% TFA, 10 mM citric acid samples were quantified.

Quantitative analysis of Phosphopeptide Enriched Samples. Quantitative LC-MS/MS was performed in singlicate (4 uL=33% of the total sample each injection) for phosphopeptide-enriched samples using a nanoAcquity UPLC system (Waters Corp) coupled to a Thermo QExactive Plus high resolution accurate mass tandem mass spectrometer (Thermo) via a nanoelectrospray ionization source. Briefly, the sample was first trapped on a Symmetry C18 300 mm Å˜180 mm trapping column for 6 min at 51/min (99.9/0.1 v/v water/acetonitrile 0.1% formic acid), after which the analytical separation was performed on a 1.7 μm Acquity BEH130 C18 75 mm Å˜250 mm column (Waters Corp). Peptides were held at 3% acetonitrile with 0.1% formic acid for 5 min and then subjected to a linear gradient from 3 to 30% acetonitrile with 0.1% formic acid over 90 min at a flow rate of 400 nL/min at 55° C. Data collection on the QExactivePlus mass-spec was performed in a data-dependent acquisition (DDA) mode following protocol of the manufacturer.

Statistics

All data are expressed as mean±SEM. Differences between groups were evaluated using two-tailed Student's t test (experimental against sham control), or in the case of multiple groups, one-way ANOVA followed by post-hoc Bonferroni test. The criterion for statistical significance is p<0.05.

TABLE 2
PCR Primers

| | | |
|---|---|---|
| Target | Forward primer | Reverse primer |

Site-directed mutagenesis

| | | |
|---|---|---|
| S276A | 5′GCGCCCCAGGGCGGTGCACCCACCA | 5′CGCTGCAGCTTGGTGGGTGCACCG |
| | AGCTGCAGCG3′ | CCCTGGGGCGC3′ |
| | (SEQ ID NO: 01) | (SEQ ID NO: 02) |
| Kaiso1 | 5GGGTTCTAGACTGAAACTAGTGACT | 5′CACAAAGCCAATGAGTCACTAGTT |
| | CATTGGCTTTGTG′3′ | TCAGTCTAGAACCC3′ |
| | (SEQ ID NO: 03) | (SEQ ID NO: 04) |
| Kaiso2 | 5′GCTCAACAACCTGACGACTAGTGAG | 5′CCCCATCGCCGTCCTCACTAGTCGT |
| | GACGGCGATGGGG3′ | CAGGTTGTTGAGC3′ |
| | (SEQ ID NO: 05) | (SEQ ID NO: 06) |
| TCF | 5′CAAATCCCTTAGAAGCAACTAGTCG | 5′GTCTTCTTCGATGGACGACTAGTTG |
| | TCCATCGAAGAAGAC3′ | CTTCTAAGGGATTTG3′ |
| | (SEQ ID NO: 07) | (SEQ ID NO: 08) |

RT-qPCR

| | | |
|---|---|---|
| KCC2 | 5′CTGACGGACTGCGAGGACGG3′ | 5′GGCTGGTGTCCATCTCCTCCTCAA3′ |
| | (SEQ ID NO: 09) | (SEQ ID NO: 10) |
| tubulin | 5′CCTGCCTTTTCGTCTCTAGCCGC3′ | 5GCTGATGACCTCCCAGAACTTGGC′3′ |
| | (SEQ ID NO: 11) | (SEQ ID NO: 12) |

ChIP Assay

| | | |
|---|---|---|
| Kaiso1 | 5′AGCTCATCCCATACTCAAACCCTG3′ | 5′GCATCTTGGAGATCTAAACTGCTA |
| | (SEQ ID NO: 13) | GC3′ |
| | | (SEQ ID NO: 14) |
| Kaiso2 | 5′TGCATACGGGATGAGGTGAGCAGC3′ | 5′CAGAACCGTGGACAGCGCCTAGCG |
| | (SEQ ID NO: 15) | 3′ |
| | | (SEQ ID NO: 16) |
| TCF | 5′CTGAGCTGTATATCACACGGTCTGC3′ | 5′TACGCTACCCAGCTGTCTCTGATTC3′ |
| | (SEQ ID NO: 17) | (SEQ ID NO: 18) |

### Example 1: 1057 Compound Screen in Primary Cortical Neurons for KCC2 Gene Expression Enhancers

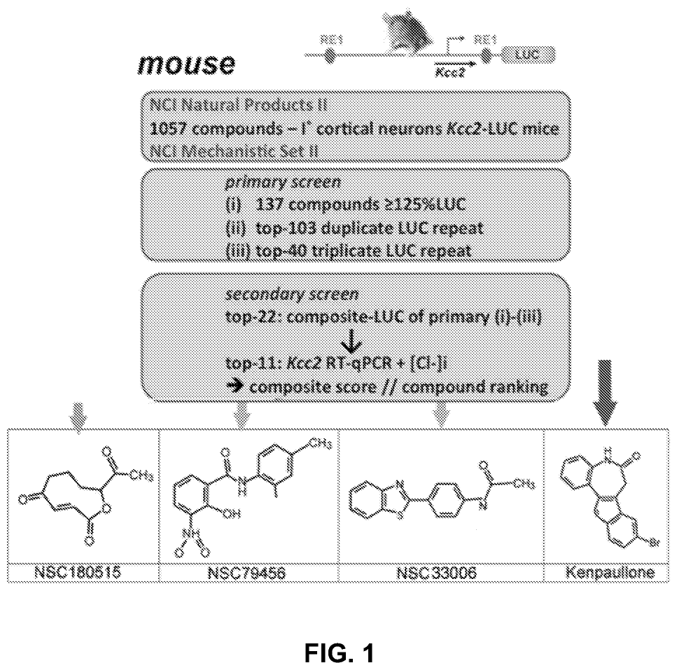

To identify KCC2 gene expression enhancing compounds, cultured primary cortical neurons were cultured from KCC2-luciferase(LUC)-knockin (KCC2-LUCki) mice (Liedtke et al., 2013, Small 9:1066-1075; Yeo et al., 2013, Proc Natl Acad Sci USA 110:4315-4320) and used LUC metrics as readout for activity of the proximal KCC2 promoter (2.5 kB (Yeo et al., 2009, J Neurosci 29:14652-14662)), which drives LUC in this mouse transgenic line. A Z′ factor of 0.94 was obtained. This strategy will not select for long-range enhancers of KCC2 gene expression that act outside the 2.5 kB core KCC2 promoter. 1057 compounds, contained in two NCI libraries (FIG. 1), related to inhibition of growth of malignantly transformed cells were screened. Iterative screening followed by measurements of KCC2 mRNA (RT-qPCR) and intracellular chloride ([Cl—]i), using clomeleon chloride indicator protein (Kuner and Augustine, 2000, Neuron 27:447-459; Yeo et al., 2009, J Neurosci 29:14652-14662), led to decreasing numbers of confirmed and re-confirmed hits, and 4 “winner” compounds were selected (FIG. 1 and Table 3).

TABLE 3
Iterative Screening Compounds

| Luciferase | RT-QPCR | Clomeleon | | | chloride | chloride | |
|---|---|---|---|---|---|---|---|
| Score | Score | Score | Total | Top 2 | [Cl—]i | reduction | Natural |
| (triplicates) | (triplicates) | (duplicates) | Score | hits | mM | (%) | Compounds |
| 1 | 5 | 5 | 11 | — | 75 => 42 | 56 | confertifoline |
| 2 | 2 | 2 | 6 | winner | 68 => 23 | 33.8 | kenpaullone |
| | | | | | | | (9-bromo-7,12- |
| | | | | | | | dihydro-indolo |
| | | | | | | | [3,2-d] |
| | | | | | | | [1]benzazepin- |
| | | | | | | | 6(5H)-one) |
| 3 | 4 | 6 | 13 | — | 75 => 43 | 57.3 | anisomycine |
| 4 | 1 | 1 | 6 | winner | 68 => 22 | 32 | NSC180515 |
| | | | | | | | [2-Acetyl-2,3,4,5- |
| | | | | | | | tetrahydrooxonine- |
| | | | | | | | 6,9-dione] |
| 5 | 3 | 4 | 12 | — | 75 => 39 | 52 | tylocrebrine |
| 6 | 6 | 3 | 15 | — | 68 => 33 | 48.5 | cantharidine |
| 7 | — | — | — | — | — | — | veratridine |
| 8 | — | — | — | — | — | — | |
| 9 | — | — | — | — | — | — | proresidor |
| 9 | — | — | — | — | — | — | ketoconazole |
| 10 | — | — | — | — | — | — | colchicine |
| Luciferase | RT-QPCR | Clomeleon | Aggre- | Top | chloride | chloride | |
| Score | Score | Score | gate | two | [Cl—]i | reduction | Mechanistic |
| (triplicates) | (triplicates) | (duplicates) | Score | hits | mM | (%) | Compounds |
| 1 | 3 | 1 | 5 | winner | 74 => 24 | 32.4 | NSC79456 |
| | | | | | | | Dimethylphenyl)- |
| | | | | | | | 2-hydroxy-3- |
| | | | | | | | nitrobenzamide] |
| 2 | 4 | 5 | 11 | — | 82 => 40 | 48.8 | 9-Deazaadenosine |
| 2 | 1 | 2 | 5 | winner | 74 => 28 | 37.8 | NSC33006 |
| | | | | | | | [N-[4-(1,3- |
| | | | | | | | benzothiazol-2- |
| | | | | | | | yl)phenyl]ace- |
| | | | | | | | tamide] |
| 3 | 2 | 3 | 8 | — | 74 => 32 | 43.2 | 4-Ipomeanol |
| 4 | 5 | 4 | 13 | — | 82 => 38 | 46.3 | 4-(1,3- |
| | | | | | | | Benzothiazol-2- |
| | | | | | | | yl)-2- |
| | | | | | | | methylaniline |
| 5 | — | — | — | — | — | — | Quinolinium, |
| | | | | | | | dichloride |
| 5 | — | — | — | — | — | — | (2E)-N-(2- |
| | | | | | | | methylphenyl)-2- |
| | | | | | | | [(3- |
| | | | | | | | nitrophenyl)methyl- |
| | | | | | | | idene]-3- |
| | | | | | | | oxobutanamide |
| 5 | — | — | — | — | — | — | Akt Inhibitor V |
| 6 | — | — | — | — | — | — | Sauzivamycin |
| 6 | — | — | — | — | — | — | 3,3,4,4- |
| | | | | | | | tetramethyl- |
| | | | | | | | oxolane-2,5-diol |

Of these, kenpaullone (KP) (9-bromo-7,12-dihydro-indolo [3,2-d] [1]benzazepin-6(5H)-one), a GSK3/CDK kinase inhibitor, was identified as one of the promising compound for further study based on its previous record of neuroprotection in translationally relevant preclinical models (Liu et al., 2016, Cell reports 14:115-128; Reinhardt et al., 2019, Stem Cell Reports 12:502-517; Skardelly et al., 2011, Neuroscience 29:543-547; Yang et al., 2013, Cell Stem Cell 12:713-726).

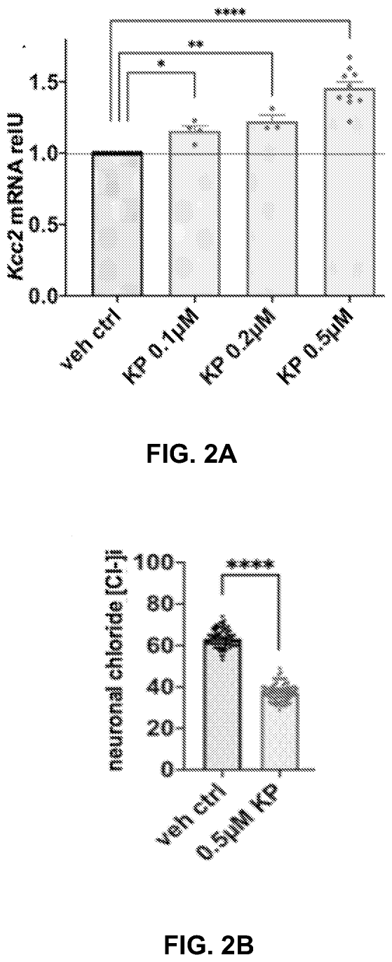

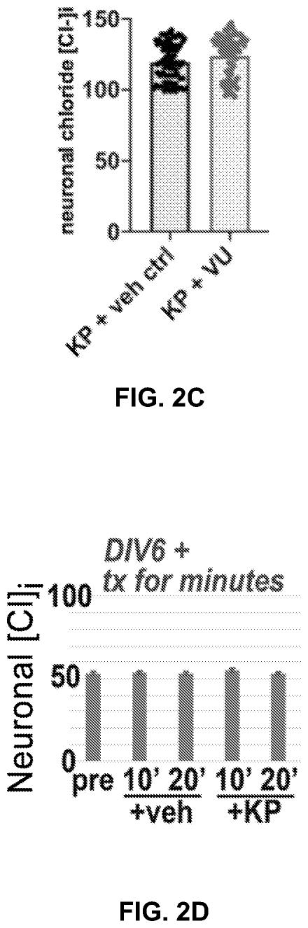

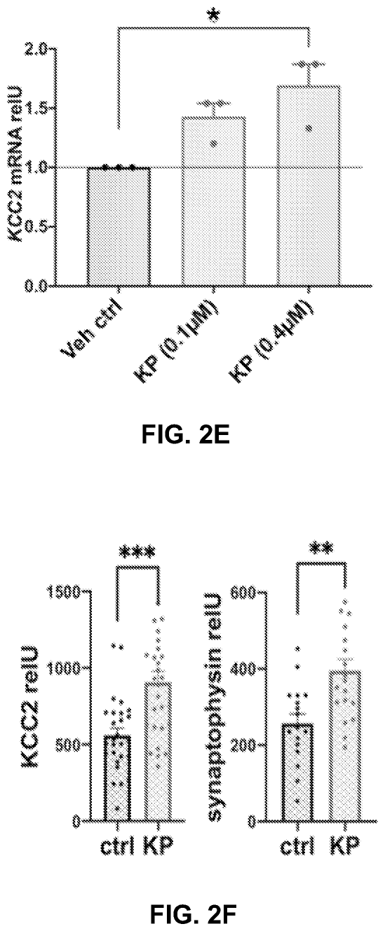

These data establish that (i) KP enhanced KCC2 gene expression in rat and mouse primary cortical neurons (FIG. 2A), (ii) this effect was dose-dependent when tested in rat neurons (FIG. 2A) and also reflected by increased protein expression as shown by KCC2 immuno-label (micrographs data not shown), (iii) in rat neurons, KP lowered [Cl—]i (FIG. 2B), (iv) this effect relied on chloride-extruding function of KCC2 transporter protein (FIG. 2C), (v) KP did not function as an enhancer of KCC2 transporter-mediated chloride efflux (FIG. 2D), (vi) KP enhanced KCC2 gene expression and KCC2 chloride extrusion function with rather rapid kinetics within 3 h (FIG. 2D); (vii) in human primary cortical neurons, KP dose-dependently enhanced KCC2 gene expression (FIG. 2E). The latter finding was accompanied by increased protein expression of KCC2 and synaptophysin (FIG. 2F), both of them co-localizing as determined by confocal imaging at DIV10 immuno-labelled for KCC2 and synaptic maturation-marker, synaptophysin, after vehicle or KP-treatment (images not shown). Synaptophysin was used as a marker of synaptic maturation and generally of a mature neuronal phenotype, which is rooted in increased expression of KCC2. These findings in rodent and human neurons indicate that the rationally designed screen identified a GSK3/CDK kinase inhibitor, KP, which enhanced KCC2/KCC2 gene expression and not KCC2-mediated chloride extrusion in CNS neurons. KP functioned as KCC2/KCC2 gene expression enhancer in mammals including humans. In human primary cortical neurons we found enhanced synaptic maturation and increased KCC2 expression, most likely causally linked.

### Example 2: KP Exhibits Analgesic and Antipruritic Activities In Vivo

In view of this discovery, in vivo analgesic effects of KP were addressed next.

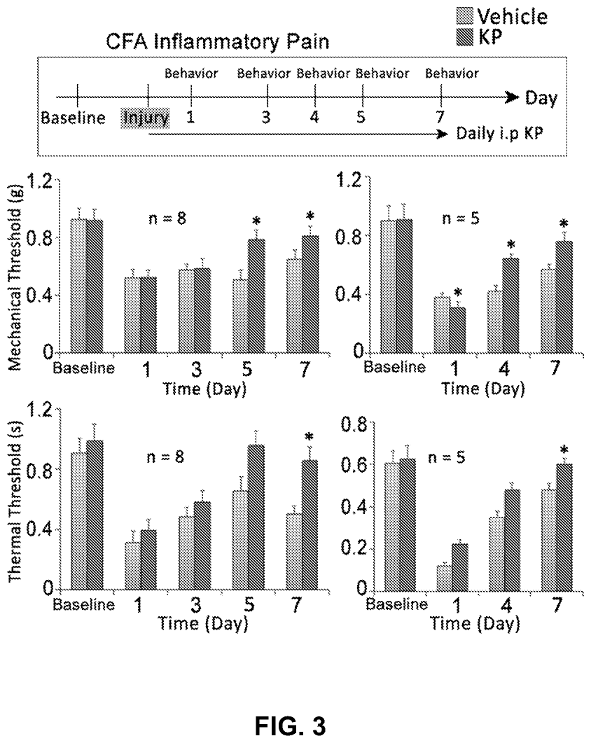

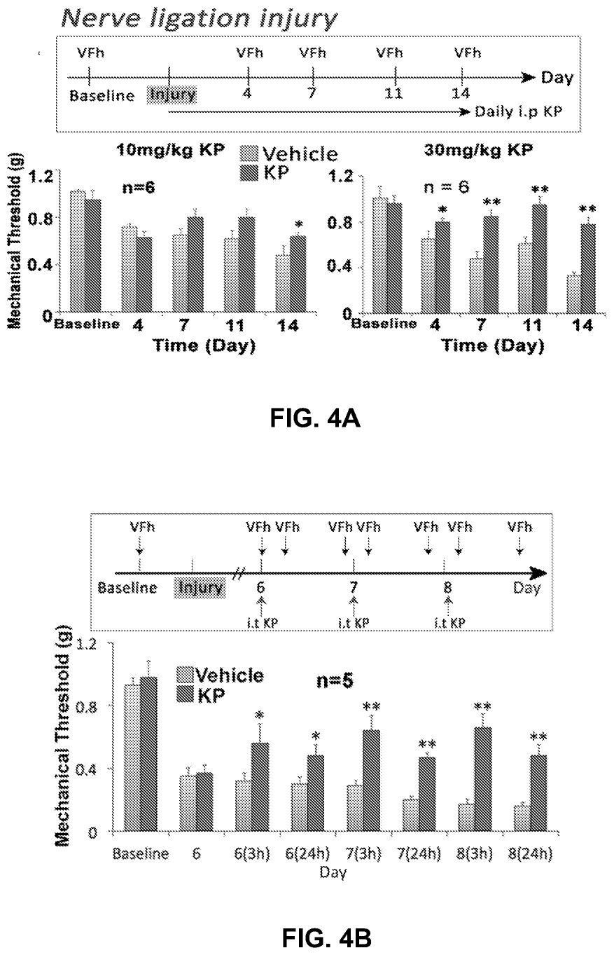

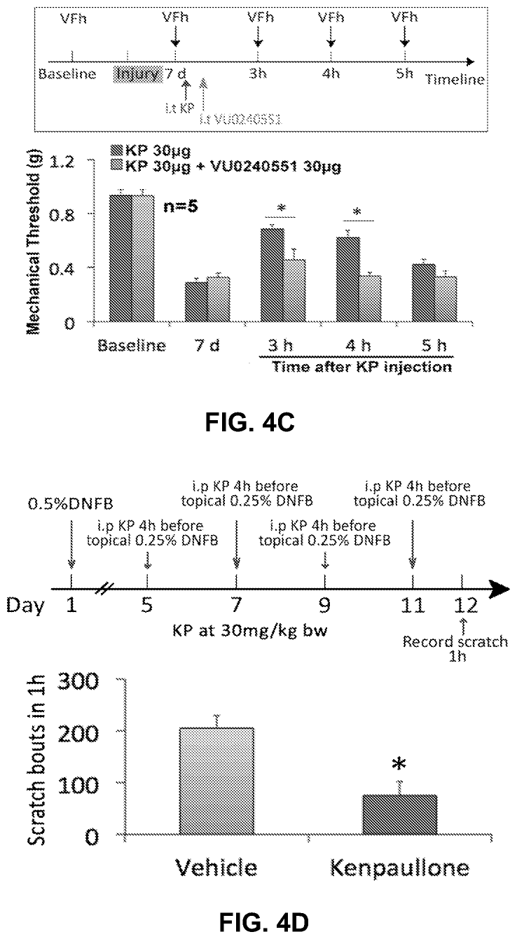

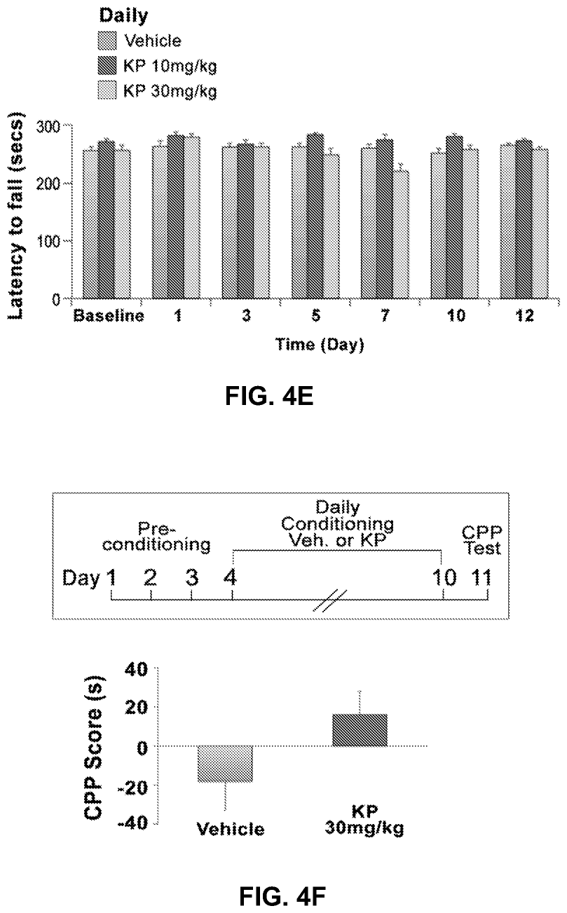

It was found that KP showed analgesic effects in two preclinical mouse models of pathologic pain: nerve injury-induced neuropathic pain, implemented by peripheral nerve constriction, and inflammatory pain, induced by peripheral tissue injection of complete Freund's adjuvans (CFA) (FIG. 3). Nerve injury- and CFA-induced behavioral sensitization (e.g., decrease in withdrawal thresholds in response to mechanical cues) were significantly reduced by KP treatment. For neuropathic pain, these effects of KP were dose-dependent, namely less accentuated at the lower dose of 10 mg/kg daily intraperitoneal (i.p) injections, and more pronounced at 30 mg/kg (FIG. 4A). Importantly, protracted analgesia, e.g. an almost complete elimination of sensitization after nerve constriction on d7-14 was observed. This finding can be interpreted as re-programming of sensitized nociception, and is rather not suggestive of selective analgesic inhibition of a pro-algesic ion channel or receptor. Thus, in vivo, KP functions as an analgesic when administered systemically in injury-related pain models.

Next, whether the analgesic effects of KP were mediated centrally at the spinal cord level was assessed. KP was injected intrathecally (i.t; 30 μg) and reduced mechanical allodynia in mice with nerve constriction injury was observed (FIG. 4B). KP was then co-applied with the KCC2 chloride transport inhibitor, VU0240551, and an elimination of the i.t analgesic effects of KP was observed (FIG. 4C), which suggests that the central analgesic effect of KP depends on KCC2-mediated chloride extrusion.

Based on these findings, KP's anti-pruritic effects were assessed because inhibitory transmission in the SCDH plays an important role in chronic pruritus (Akiyama et al., 2015, Pain 156:1240-1246.; Bourane et al., 2015, Science 350:550-554; Braz et al., 2017, Prog Brain Res 231:87-105; Braz et al., 2014, J Clin Invest 124:3612-3616; Koch et al., 2018, Annual review of physiology 80:189-217; Mishra and Hoon, 2015, Handbook of experimental pharmacology 226:151-162). In a 2,4-dinitrofluorobenzene (DNFB) chronic contact dermatitis model (Zhang et al., 2015, Cell Physiol Biochem 35:1023-1033), daily i.p injections of KP (30 mg/kg) significantly reduced robust scratching behavior of sensitized sites (FIG. 4D).

With these beneficial effects of KP on pathologic pain and itch, whether KP had undesirable effects on the CNS in terms of sedation, impairment of motor stamina, balance and coordination was assessed. Rotarod testing (Heyser et al., 2013, Physiol Behav 118:208-211) of KP-treated mice (10, 30 mg/kg; i.p.) revealed that KP does not induce unwanted side effects (FIG. 4E). Conditioned place preference was used to test whether KP (30 mg/kg; i.p.) can trigger brain reward mechanisms (Jackson et al., 2019, Psychopharmacology, 236(12):3593-3599; Sora et al., 1998, Proc Natl Acad Sci USA 95:7699-7704), and there were no such effects (FIG. 4F). Thus, KP functions as an analgesic and antipruritic in preclinical mouse models and does not cause unwanted side effects including reward mechanisms, sedation, lack of coordination, and reduced stamina. In pathologic pain models, KP acts centrally to mediate analgesic effects, and thus relies on KCC2 chloride extrusion.

### Example 3: KP Renormalizes E_{GABA} in Spinal Cord Dorsal Horn (SCDH) by Increasing KCC2 Expression and Function

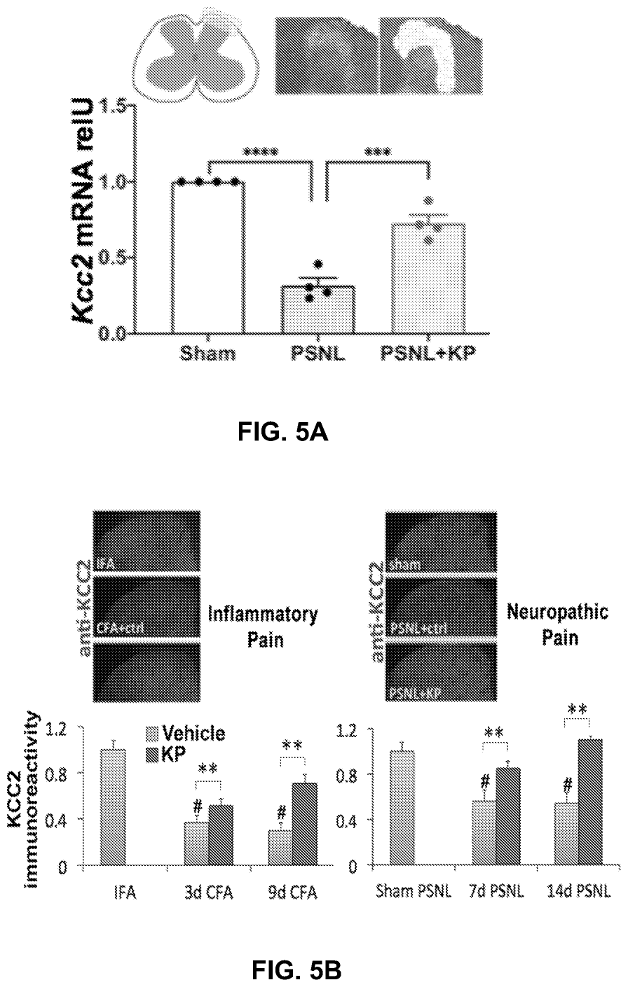

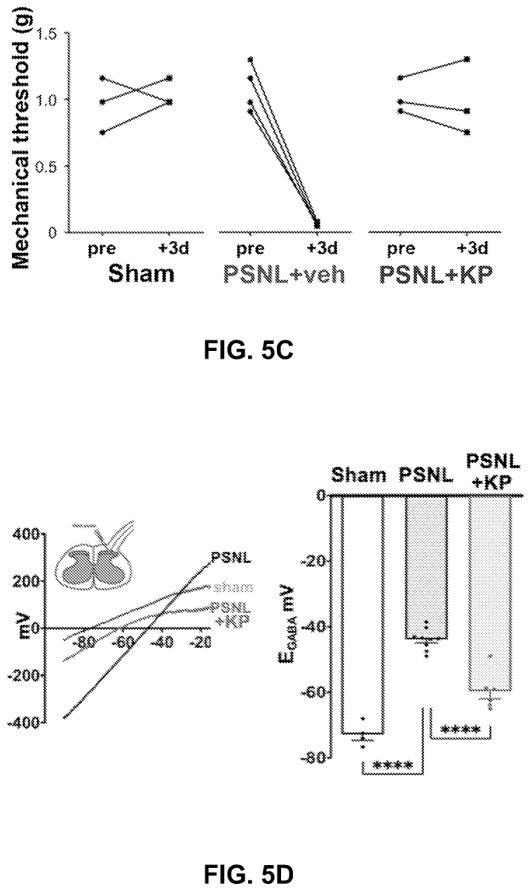

With the aforementioned findings and in view of the SCDH as the likely site of analgesic action of KP (Coull et al., 2003, Nature 424:938-942; Gagnon et al., 2013, Nat Med 19:1524-1528), SCDH KCC2 expression and function in nerve injury and response to KP was assessed. It was found that in the SCDH, KP repaired attenuated KCC2 expression caused by nerve injury, at both the mRNA and protein levels (FIG. 5A). To measure KCC2 mRNA, SCDH Rexed-layers I-II were microdissected with laser capture followed by RT-qPCR. SCDH KCC2 protein expression was measured morphometrically after KCC2 immunolabeling using SCDH laminae I-II as the region-of-interest (FIG. 5B).

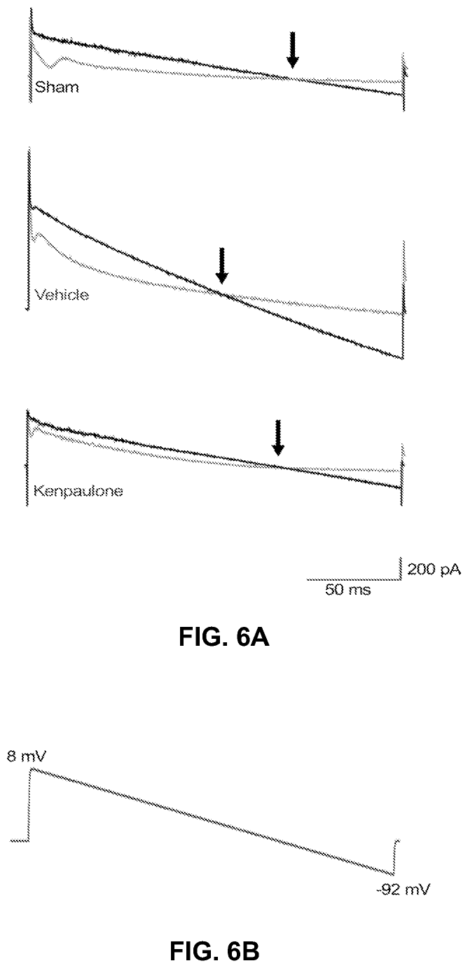

Next, whether repair of attenuated KCC2 expression was associated with re-normalization of chloride reversal potential after application of GABA (EGABA) was assessed. Prior to interrogation of spinal cord slice preparations at 72 h post-injury, robust mechanical allodynia of young mice by peripheral nerve constriction injury and its almost complete behavioral reversal by systemic treatment with KP at 30 mg/kg was shown (FIG. 5C). Next, in spinal cord slice preparations derived from these animals, SCDH lamina-II neurons were investigated by perforated patch-clamp (FIG. 5D, FIG. 6A and FIG. 6B). These data reveal that the greatly more positive, more excitable EGABA reversal potential in nerve-injury mice was renormalized toward negative, more stable values in KP-treated animals (FIG. 5D). This re-normalization of EGABA in SCDH lamina-II neurons is consistent with repair of KCC2 expression in SCDH in KP-treated mice. Thus, in a nerve injury model, the central analgesic action of KP relies on the enhanced gene expression of KCC2 and re-normalization of EGABA in pain-relaying neurons in the superficial spinal cord.

### Example 4: Cellular Mechanism of Action in Neurons: GSK313ß→δ-cat→Kaiso→KCC2

These findings set up a compelling rationale to deconstruct the cellular mechanism of action of KP that accounts for its effects in SCDH neurons and thus its sensory effects. These studies were conducted in primary cortical neurons because 1) these neurons were used for the initial screen, 2) there was rodent-human similarity in terms of the effects of KP on KCC2 KCC2 gene expression, and 3) the effects of KP on KCC2 gene expression and KCC2 chloride transporter function in these neurons were highly similar to our findings in SCDH lamina-II neurons, which cannot be cultured as readily for mechanistic cellular studies.

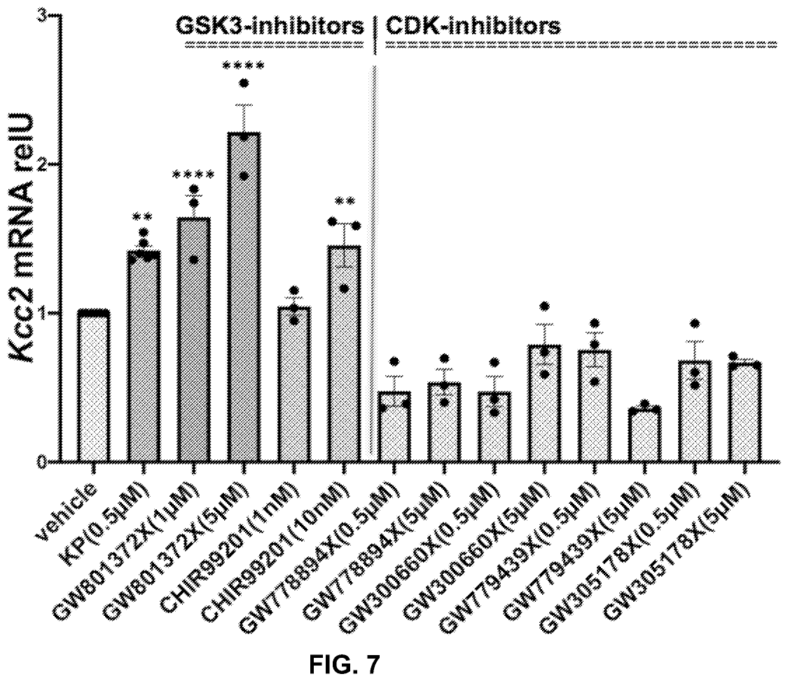

First, the question of whether KP inhibiting GSK3ß or CDKs is responsible for increasing expression of KCC2 was addressed. Using a set of GSK3 inhibitors different from KP, increased expression of KCC2 was shown. In contrast, a suite of CDK-inhibitory compounds did not increase (but decreased) expression of KCC2 (FIG. 7).

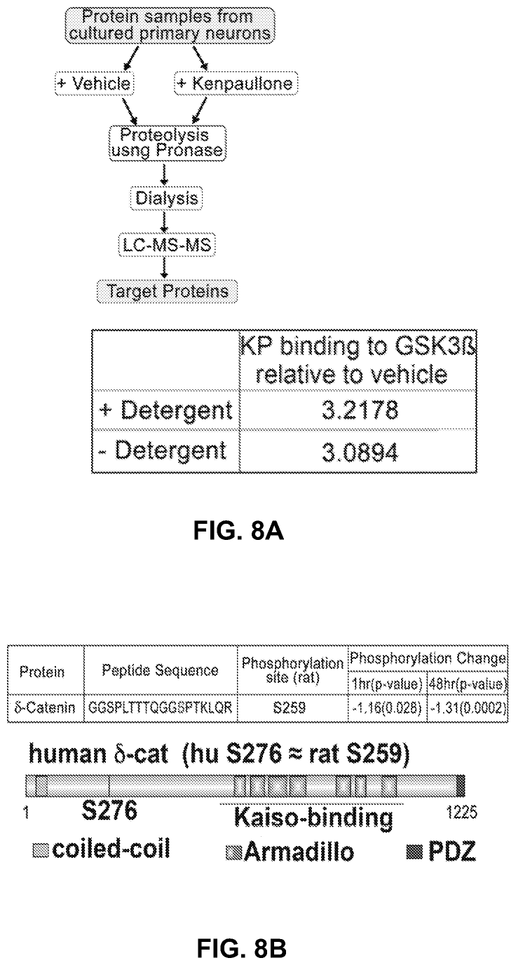

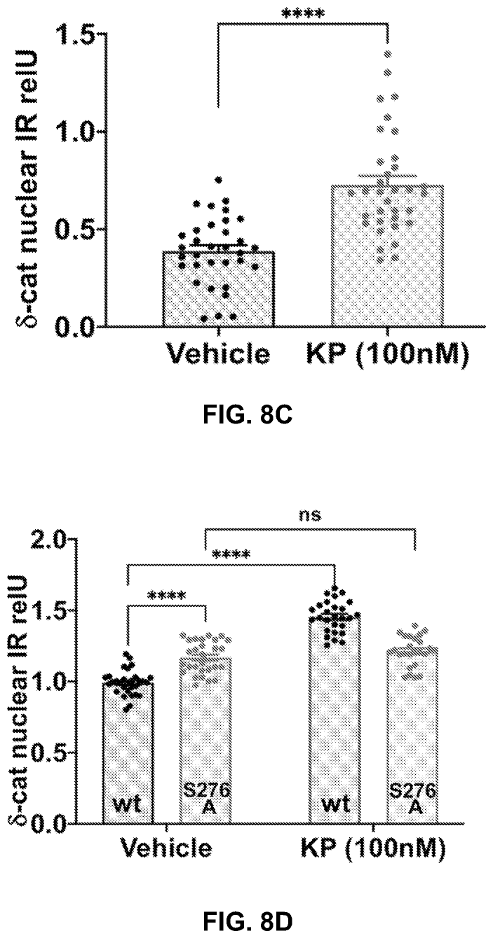

A drug affinity responsive target stability (DARTS) assay (Lomenick, et al., 2009, Proc Natl Acad Sci USA 106:21984-21989) was used to study direct binding of KP to GSK3ß (FIG. 8A) in primary cortical neurons. The findings were consistent with previous reports in cell lines (Knockaert, et al., 2002, The Journal of biological chemistry 277:25493-25501) yet novel in primary neurons. Of note, other known kinase targets of KP such as CDKs were not identified in our unbiased DARTS assay. This confirms the result with inhibitor compounds that it is the GSK3ß-inhibitory function of KP that is responsible for its KCC2 KCC2 gene expression enhancing property in neurons. To identify kinase targets of GSK3ß that are differentially phosphorylated in response to KP, unbiased phosphoproteomics assays in rat primary cortical neurons were used. The neuronal catenin, δ-catenin (CTNND2; δ-cat) was identified (Bareiss, et al., 2010, J Neurosci Res 88:2350-2363; Kosik, et al., 2005, Trends in cell biology, 15:172-178). It was found that the serine at position 259 was one site at which differential phosphorylation occurred short-term (1 h) and persisted long-term (24 h) in response to KP (FIG. 8B). The respective residue in human δ-cat is S276. ß-catenin (ß-cat) could also be a GSK3ß kinase target, yet ß-cat was not significantly differentially phosphorylated. δ-cat(S276) is found in phosphosite.org and has been previously described (Herskowitz, et al., 2010, J Proteome Res 9:6368-6379), but its identification in primary neurons is novel. Catenin phosphorylation is known to facilitate its own intracellular degradation via ubiquitination (Orford, et al., 1997, The Journal of biological chemistry 272:24735-24738; Oh, et al., 2009, The Journal of biological chemistry 284:28579-28589). To examine whether non-phosphorylated δ-cat traffics to the neuronal nucleus, specific δ-cat immunolabeling followed by confocal microscopy and morphometry was conducted. The results indicate that KP treatment of primary neurons enhanced δ-cat nuclear transfer (FIG. 8C and FIG. 8D). Given the known binding of ß-cat/δ-cat, ß-cat was immunolabeled and obtained a similar result (FIG. 9). Neuronalization of N2a cultured cells in response to KP (0.2 μM for 3 days) was conducted and increased process formation and expression of neuronal ii-tubulin was observed on micrograph images (data not shown). DAPI stain was used to define nuclear compartments. kenpaullone enhances neuronal differentiation. These results corroborated the δ-cat nuclear transfer results.

To investigate the relevance of δ-cat residue S276, N2a neural cells were studied because these cells transfect at higher efficiency than primary cortical neurons. In our cultures, N2a cells expressed neuronal ß_{III}-tubulin in elongated processes as determined by immune-labeling as an indicator of their neuronal differentiation. Furthermore, nuclear transfer of δ-cat was significantly enhanced upon KP treatment in N2a cells transfected with human δ-cat(WT) (micrograph images not shown). This increase in nuclear transfer was very similar to the trafficking we recorded in primary cortical neurons, thus validating the cell line. To determine the function of δ-cat residue S276 we then transfected δ-cat(S276A), which resulted in significantly increased nuclear transfer of δ-cat(S276A), using morphometry. Of note, there was no increase of nuclear transfer of δ-cat(S276A) upon KP treatment (FIG. 8C and FIG. 8D).

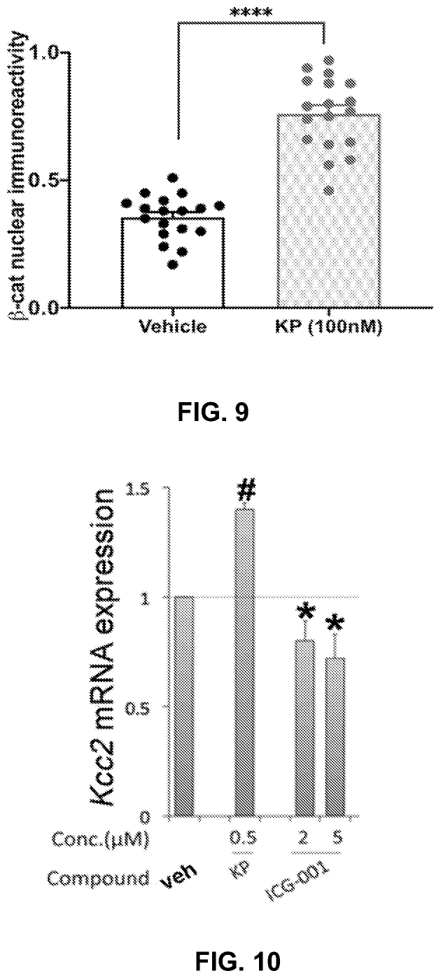

Thus, δ-cat(S259/S276) (rat/human) is very likely a relevant phosphorylation site in δ-cat and a GSK3ß kinase target in neurons. Inhibition of GSK3ß or rendering S276 phosphorylation-resistant enhances nuclear transfer of δ-cat, also of its binding partner ß-cat. Furthermore, a selective catenin-inhibitor attenuated KCC2 gene expression dose-dependently (FIG. 10). Thus, catenins enhance KCC2 gene expression in neurons.

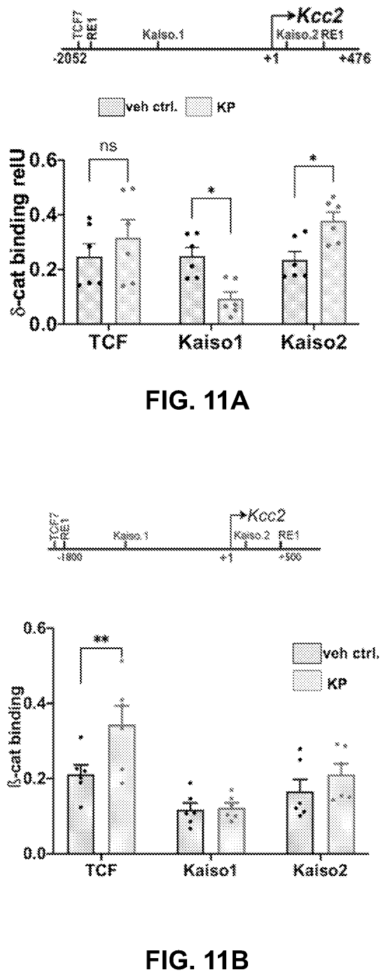

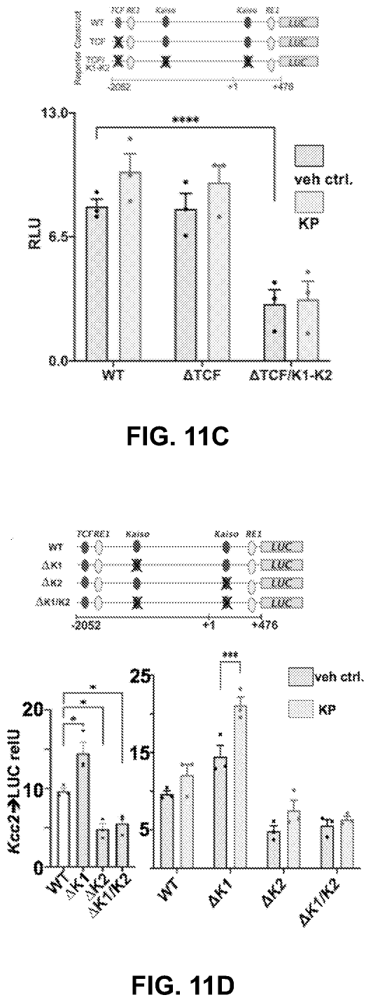

Consequently, catenins effects on the KCC2 promoter was explored. Two Kaiso binding sites (potential sites for delta-cat (Rodova, et al. 2004, Mol Cell Biol 24:7188-7196; Dai, et al. 2011, Cancer science 102:95-103) were identified in the KCC2 proximal promoter using computational methods (Aerts, et al., 2005, Nucleic Acids Res 33:W393-396). Two Kaiso binding sites bracketed the transcriptional start site (TSS) of the KCC2 gene (FIG. 11A). In rat primary cortical neurons, δ-cat was bound to both Kaiso sites in the KCC2 promoter (FIG. 11A). Interestingly, treatment with KP inhibited the binding of delta-cat to the upstream site. Binding of δ-cat to the site 3′ to the TSS was enhanced, suggesting that the upstream site functions as repressor and the 3′ site as enhancer. Beta-cat bound to a TCF (T-cell factor) DNA-binding site (Sakamoto, et al. 2000 The Journal of biological chemistry 275:32871-32878) close to the 5′ RE-1 site within the KCC2 promoter (FIG. 11B and FIG. 11C) (Yeo, et al., 2009, J Neurosci 29:14652-14662), and treatment of cells with KP significantly increased this interaction, indicative of enhancement.

Promoter expression constructs were built with rationally-targeted deletions to interrogate the effects of the Kaiso- and TCF-binding sites on activity of the KCC2 promoter and to determine if this activity was regulated by KP. For ease of transfection, key to this method, N2a neural cells were again used. The 5′ and 3′ delta-cat Kaiso binding sites functioned in repressive and enhancing manners, respectively (FIG. 11D). Presence of the 3′ delta-cat Kaiso binding site and absence of the 5′ site led to significantly enhanced activity of the KCC2 promoter upon treatment with KP. Deletion of both Kaiso sites led to markedly reduced promoter activity and non-responsiveness of the construct to KP. Deletion of the TCF binding-site from the KCC2 promoter did not change KCC2 promoter activity or its response to KP treatment (FIG. 11B and FIG. 11C). However, the triple-deletion of both Kaiso sites and the TCF site rendered the construct minimally active and completely non-responsive to KP.

These data suggest that δ-cat, a kinase target of GSK3ß in CNS neurons, traffics to the nucleus increasingly upon GSK3ß inhibition. In the nucleus, δ-cat interacts with the KCC2 promoter to enhance KCC2 expression via two Kaiso DNA-binding sites. ⊕-cat co-traffics to the nucleus with δ-cat in response to KP. Beta-cat is not a significant neuronal GSK3ß kinase target, and plays an ancillary role in enhancement of KCC2 gene expression.

### Example 5: δ-Cat Spinal Transgenesis is Analgesic in Nerve Constriction Injury

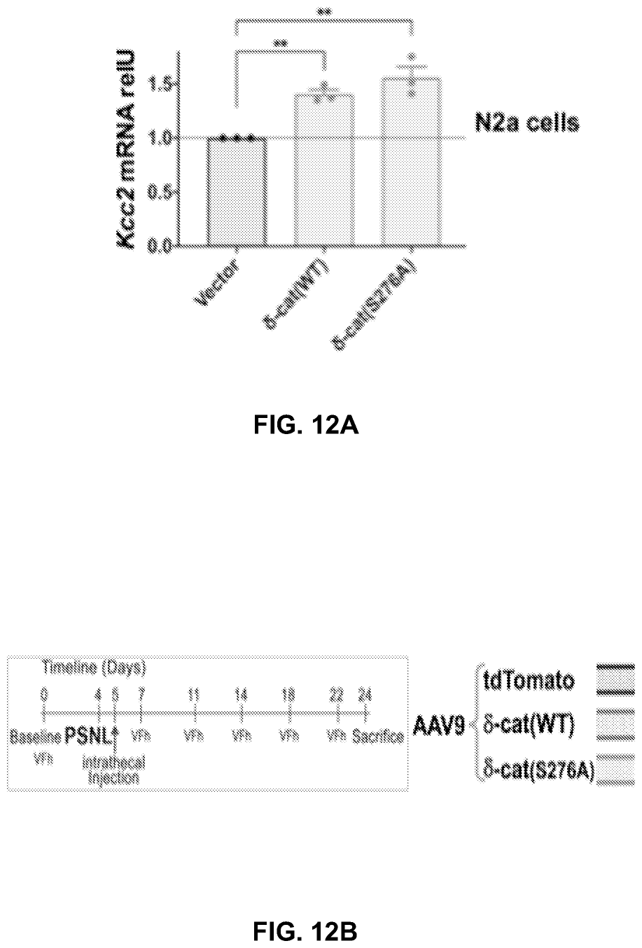

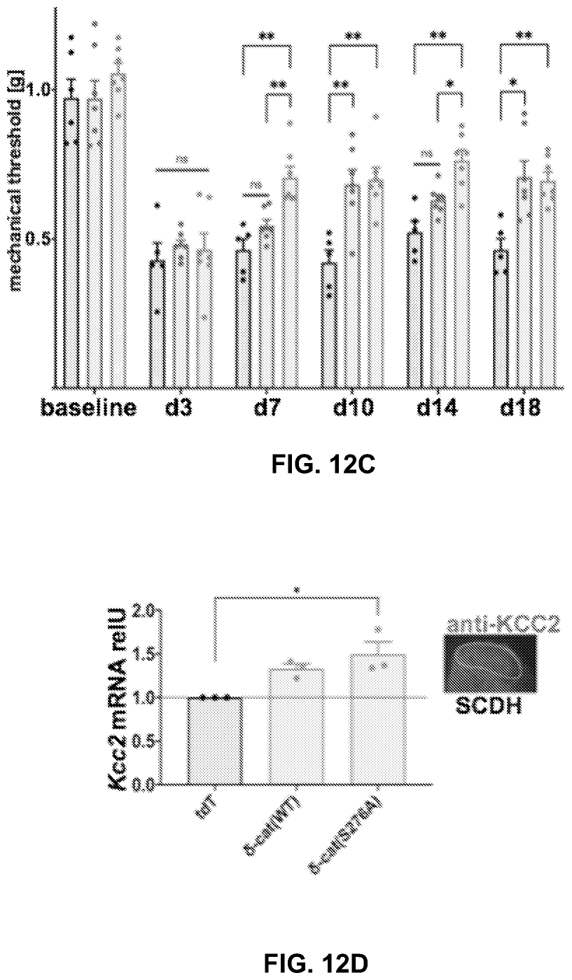

Next, the question of whether δ-cat, when expressed as a spinal transgene in sensory relay neurons, will facilitate analgesia in nerve constriction injury was assessed. A δ-cat transgene increases KCC2 expression in N2a neural cells was observed, and that KCC2 expression levels were slightly elevated when using δ-cat(S276A) (FIG. 12A). Thus, human δ-cat transgenes mimic the effects of KP in a mouse neural cell line.

AAV9 vectors harboring human δ-cat and δ-cat(S276A), driven by the minimal human neuronal synapsin promoter (huSyn (Liu, et al. 2008, BMC Biotechnol 8:49) were constructed. AAV9 and huSyn were used because in a previous in-depth study, AAV9 harboring fluorescent reporter driven by huSyn, upon i.t injection, readily transduced spinal neurons and spared DRG primary afferent neurons (Haenraets, et al., 2017, Journal of neurochemistry 142:721-733). Synapsin-tdTomato was used as control and injected 5×10⁹ viral genomes (5 μL; i.t) of each construct. Assessment of tdTomato fluorescence 3d post injection revealed spinal transgenesis that was evenly manifesting in the SCDH (FIG. 12B), in keeping with the above-mentioned previous study (Haenraets, et al., 2017, Journal of neurochemistry 142:721-733). Mechanical withdrawal was measured thresholds after nerve constriction injury (PSNL) (FIG. 12B and FIG. 12C). Significantly less sensitization for δ-cat (S276A) was found, with a protracted start of effect on d7 post-injection, and a sustained benefit until the end of experiment at d18 (FIG. 12C). δ-cat(S276A) showed increased effect vs. δ-cat(WT) on d7 and d14. δ-cat(WT) was significantly effective on d11, d18. In accordance with behavioral findings, KCC2 mRNA in microdissected SCDH was significantly increased in δ-cat(S276A) (FIG. 12D). For S-cat(WT), KCC2 mRNA abundance was elevated but not to significant levels likely because of viral transduction of only a fraction of sensory relay neurons in the SCDH. Thus, δ-cat(S276A) spinal transgenesis via AAV9 is sufficient to evoke analgesia after nerve constriction injury, to lesser degree also with δ-cat(WT). Importantly, δ-cat(S276A) spinal transgenesis was accompanied by increased expression of KCC2 in the SCDH. KCC2 protein, as measured by immunolabeling and morphometry in SCDH layers-I/II was significantly increased in animals injected with δ-cat viral vectors. These findings suggest that our proposed mechanism of KCC2 gene expression enhancement by KP, via nuclear action of δ-cat at the KCC2 promoter, which we established in primary neurons and neural cells, remained valid in a nerve constriction preclinical pain model in mice. Moreover, our discovery points toward a genetically-encoded approach that can be developed for translation into clinical use as an alternative or complement to KP-related small molecules.

Discussion

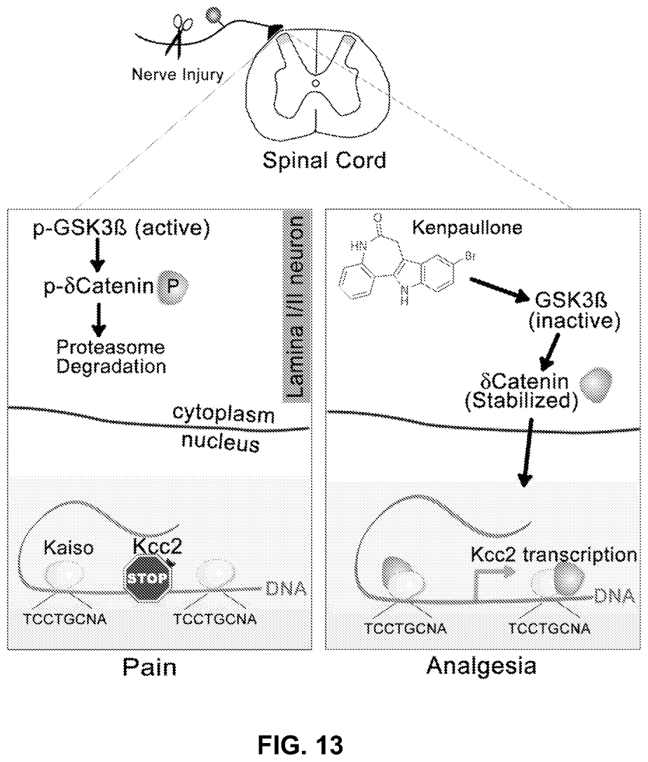

Evidence that reduced expression of the neuronal chloride extruding transporter, KCC2, contributes to a diversity of CNS diseases led us to seek a small molecule which increased its expression. Using measures of KCC2 promoter activity, KCC2 mRNA abundance, and [Cl—]i, an unbiased screen of two NCI libraries containing 1057 compounds that inhibit growth of transformed cells was conducted. This screen identified kenpaullone (KP), a GSK3/CDK kinase inhibitor with neuroprotective properties (Liu et al., 2016, Cell reports 14:115-128; Reinhardt et al., 2019, Stem Cell Reports 12:502-517; Schultz et al., 1999, J Med Chem 42:2909-2919, Skardelly et al., 2011, Neuroscience 29:543-547; Yang et al., 2013, Cell Stem Cell 12:713-726; Zaharevitz et al., 1999, Cancer research 59:2566-2569). These studies of KP revealed at least the following principal findings: 1) KP enhances KCC2 KCC2 gene expression in a concentration-dependent manner and lowers [Cl—]i in cultured mouse, rat, and human neurons; 2) Systemic administration of KP to mice attenuates measures of nerve injury pain and chronic itch in preclinical models; 3) Intrathecal administration of KP to mice attenuates nerve injury pain depending on spinal KCC2 chloride transporter activity; 4) Systemic administration of KP to mice with nerve injury enhances KCC2 gene expression in SCDH neurons and shifts the GABA-induced chloride reversal potential to more negative and electrically stable measures; and 5) The mechanism by which KP enhances KCC2 gene expression is by binding to and inhibiting GSK3ß, inhibiting phosphorylation of δ2-cat at position S259 in rat (S276 in human), which increases nuclear transfer of δ2-cat. In the nucleus, δ2-cat binds to and enhances the KCC2 promoter via two Kaiso-binding regulatory sites. Transferring this mechanism from neural cells to the live animal, spinal transgenesis of δ2-cat(S2276A) attenuates nerve injury pain in mice. Thus, KP and the new GSK3ß [symbol] δ2-cat [symbol] Kaiso [symbol] KCC2 signaling pathway may represent a strategic bridge-head for therapeutics development for treatment of pathologic pain. Beyond pain, this could also apply to other neurologic and mental health conditions in which restoration of KCC2 function is important, such as epilepsy, traumatic spinal cord and brain injury, neurodegeneration and neurodevelopmental disorders. This proposed analgesic mechanism is summarized in FIG. 13.

Compounds from two NCI libraries that can interfere with gene regulation in CNS neurons were identified. A substantial number of the molecules contained in these libraries can regulate growth of rapidly dividing cells by interfering with chromatin and via additional epigenetic mechanisms. Therefore, these compounds can act similarly in non-dividing CNS neurons such as primary developing cortical neurons. These neurons helped identify compounds that can activate the KCC2 promoter, and KP was selected as the “winner” compound for in-depth exploration. Though KP is predicted to have multiple targets in CNS neurons, the data shown herein provide evidence that KP binds to neuronal GSK3ß and not to CDKs. In addition, it is known that KP inhibits GSK3ß with highest potency from amongst known targets (Knockaert et al., 2002, The Journal of biological chemistry 277:25493-25501; Kunick et al., 2004, Bioorg Med Chem Lett 14:413-416; Schultz et al., 1999, J Med Chem 42:2909-2919). The results demonstrated that GSK3ß inhibition by KP directly upregulates KCC2 gene expression via the δ-catenin-Kaiso pathway. This mechanism of a GSK3-inhibitory compound has not been reported previously. Of note, this novel concept held true in primary human neurons in which KP also enhanced synaptic maturation. Although KP can inhibit other kinases, the data suggest that inhibition of GSK3ß and subsequent enhancement of KCC2 gene expression via S-catenin are very important, perhaps dominant mechanisms of action of KP as it attenuates pathologic pain. Additionally, S-cat-Kaiso likely affects multiple neuronal genes, but the data suggest that enhanced KCC2 gene expression and KCC2 function are the major analgesic effector mechanisms of KP. Another argument, mechanistically weaker but translationally relevant, is the absence of unwanted effects of our KCC2 expression-enhancing strategy on choice behavior, motor stamina, and coordination. Effective targeting of multiple pathways would likely impact these behaviors. However, the behavioral profile for KP was similarly benign as other KCC2 expression-enhancing compounds (Gagnon et al., 2013, Nat Med 19:1524-1528).

The screening strategy addressed a fundamental problem of pathological pain. This vastly unmet medical need, rooted in its chronicity, is driven by genetic reprogramming, which results in a maladaptive phenotype (Bai et al., 2015, Translational research:the journal of laboratory and clinical medicine 165:177-199; Doyon et al., 2013, Expert review of neurotherapeutics 13:469-471; Kuner, 2010, Nat Med 16:1258-1266; Liang et al., 2015, Epigenomics 7:235-245; Sommer, 2016, Science 354:588-592). A key event is attenuated expression of KCC2 because of its relevance for inhibitory transmission in pain-relevant neural circuits (Coull et al., 2003, Nature 424:938-942; Gagnon et al., 2013, Nat Med 19:1524-1528; Kahle et al., 2014, JAMA neurology 71:640-645; Li et al., 2016, Cell reports 15:1376-1383; Mapplebeck et al., 2019, Cell reports 28:590-596 e594; Price et al., 2005, Curr Top Med Chem 5:547-555). This has also been postulated in other pathologic conditions of the CNS as a general pathogenic feature (Hwang and Zukin, 2018, Current opinion in neurobiology 48:193-200; Lardenoije et al., 2015, Progress in neurobiology 131:21-64). Regulation of KCC2 gene expression by GSK3ß and its kinase target δ-cat is a novel insight of the study described herein. This concept will permit rational exploration of links between [symbol] GSK3ß [symbol] δ-cat and attenuated KCC2 KCC2 gene expression and the resulting malfunction of inhibitory neurotransmission in several other relevant neurologic and psychiatric conditions such as Alzheimer's Disease and other neurodegenerative diseases, psychoses, traumatic brain/spinal cord injury, Rett Syndrome, Autism Spectrum Disorders, and epilepsy (Boulenguez et al., 2010, Nat Med 16:302-307; Chen et al., 2018, Cell 174:521-535 e513; Ferando et al., 2016, Nature neuroscience 19:1197-1200; Freund and Meskenaite, 1992, Proc Natl Acad Sci USA 89:738-742; Huberfeld et al., 2007, The Journal of neuroscience:the official journal of the Society for Neuroscience 27:9866-9873; Hyde et al., 2011, J Neurosci 31:11088-11095; Kahle et al., 2014, EMBO Rep 15, 766-774; Tang et al., 2016, Proc Natl Acad Sci USA 113:751-756; Tao et al., 2012, J Neurosci 32:5216-5222). KP was selected because of its previously reported neuroprotective properties for spinal motoneurons, brainstem auditory relay neurons, and hypoxia-injured hippocampal neurons (Liu et al., 2016, Cell reports 14:115-128; Reinhardt et al., 2019, Stem Cell Reports 12:502-517; Skardelly et al., 2011, Neuroscience 29:543-547; Teitz et al., 2018, The Journal of experimental medicine 215:1187-1203; Winkelmann et al., 2015, Cell death & disease 6:e1776; Yang et al., 2013, Cell Stem Cell 12:713-726). There was a unifying mechanism of KCC2 expression enhancement by KP in these previous studies. Neuroprotective properties for a novel analgesic are welcome because chronic pain is associated with non-resolving neural injury mediated by neuroinflammation (Ji et al., 2018, Anesthesiology 129:343-366). Repurposing a GSK3ß-inhibitory compound as an analgesic reprogramming compound that upregulates KCC2 expression links chronic pathologic pain to neurodegeneration at both the basic science and translational neuroscience levels.

The work described herein characterized a novel strategy to treat chronic pathologic pain and also increases our basic understanding of pain and sensory transduction. With a focus on the enhanced expression of KCC2 in neurons as an analgesic strategy, a compound was utilized as a genomic reprogramming agent that reverts the expression of a key dysregulated gene. Of practical/translational importance, it was demonstrated that KP is non-sedative and does not affect motor stamina, coordination, or choice behavior. Furthermore, validated KP was targeted in human primary neurons. In aggregate, a repurposed compound, KP, was identified and a genetically-encoded cellular signaling [symbol] pathway, GSK3ß [symbol] δ-cat [symbol] Kaiso [symbol] KCC2, which were not previously known to up-regulate KCC2. Both of these KCC2-enhancing approaches can be developed translationally into clinical neuroscience applications.

The finding that KP acts as an anti-pruritic in a chronic contact dermatitis model is novel and suggests that defective expression of KCC2 in the SCDH plays an important role in chronic inflammatory itch. This finding requires additional research to further elucidate the underlying cellular and neural circuit mechanisms. Validation in additional itch models will also facilitate translation to the clinic.

The approach as described herein for targeting of pain and itch is innovative. Enhanced KCC2 gene expression, based on KP treatment or δ-cat transgenesis as presented here, will complement direct enhancement of KCC2 chloride extrusion in targeting pathologic pain. Complementary use will help overcome recalcitrant lack of expression and function of KCC2 in pain relay neurons, as might be expected in clinical cases of “refractory” chronic pain. Clinical combination use of KCC2 expression enhancers with analgesic compounds that have different mechanisms of action will be advantageous as renormalized inhibitory transmission will cause improved effectiveness of other compounds, such as gabapentinoids.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents form part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise.

The present disclosure shall control in the event there are any disparities between any definitions and/or description found in the cited references.

Additional Sequences:

Homo sapiens catenin delta 2 (CTNND2), transcript variant/isoform 1
(Acc. No.: NM_001332.4).
SEQ ID NO: 20
GCACCCGGGGCCGGTTTCGGCGGCGGGGCGGACGCTCCCTCACTAGCCGGGCGCCCGGGCCGGCCGCC
GAGGTTTAGGTGGAGGCTGAGGCTGCCGCGCGCCGCGGGAGGAGCCTCGCCCTCGGCGGCTGAGGCGG
CGGCGGCACAGGTGGCGCGGGCCGCGCGCGGGGCGCAGCTCGGGAGCGCTCGGCGCCGGGCGCCGCGG
CGGCCCCAGGCTCGCGCCCGCGGCGACAGCCGGCTGAGCGGAGCTGCGGGCGCGGCGGCTGCTCTCTC
AGCCCGGCCGGCTGGCGGCGGCGCCCGGAGCCCCGAGGAGCCCGCAGGAGCCCGTGGAGCCCGCGGAG
CCGGGCAGGGGGCGCTGCGCTCGCCGAGCTCGGAGGGGCCGCCGGGCCGGGCGCTGCGCACTCGCGTC
GGGAGCCGCCTCTCGCCCGCGGCGCTCGCCCCTGCCGCCCGCCAGCATCCCTTGTCCCGCGGCCGCGC
TCAGACAACAAAAGCGGAAGATGCTGCAGTTGGGCAAGGTCAGGACCTTGCCCTGAAGCCGGGCGGCG
CCGCGCACGCCTCTTCCCGGACTGAGGAGCTGTCGCCGGCGGAGGGTGCATGTTTGCGAGGAAGCCGC
CGGGCGCCGCGCCTTTGGGAGCTATGCCTGTTCCAGACCAGCCTTCATCAGCCTCAGAGAAGACGAGT
TCCCTGAGCCCCGGCTTAAACACCTCCAACGGGGATGGCTCTGAAACAGAAACCACCTCTGCCATCCT
CGCCTCAGTCAAAGAACAGGAATTACAGTTTGAAAGGCTGACCCGAGAGCTGGAGGCTGAACGGCAGA
TCGTAGCCAGCCAGCTGGAGCGATGCAAGCTCGGATCCGAGACTGGCAGCATGAGCAGCATGAGTTCA
GCAGAAGAGCAGTTTCAGTGGCAGTCACAAGATGGTCAAAAAGATATCGAAGATGAGCTTACAACAGG
TCTCGAGCTGGTGGACTCCTGTATTAGGTCACTACAGGAATCAGGAATACTTGACCCACAGGATTATT
CTACAGGTGAAAGGCCCAGCCTGCTCTCCCAGAGTGCACTTCAGCTCAATTCCAAACCTGAAGGGTCT
TTCCAGTATCCGGCCAGCTACCATAGCAACCAGACCCTGGCCCTGGGGGAAACCACCCCTTCACAGCT
CCCGGCCCGAGGCACACAAGCCCGAGCTACGGGCCAGAGCTTCAGCCAGGGCACGACCAGCCGCGCCG
GCCACCTGGCGGGGCCCGAGCCCGCGCCGCCGCCGCCGCCGCCGCCGCGGGAGCCGTTCGCGCCCAGC
CTGGGCAGCGCCTTCCACCTGCCCGACGCGCCGCCCGCCGCCGCCGCCGCCGCGCTCTACTACTCCAG
CTCCACGCTGCCCGCGCCGCCGCGCGGGGGCTCCCCGCTGGCCGCGCCCCAGGGCGGTTCGCCCACCA
AGCTGCAGCGCGGCGGCTCGGCCCCCGAGGGCGCCACCTACGCCGCGCCGCGCGGCTCCTCGCCCAAG
CAGTCGCCCAGCCGCCTGGCCAAGTCCTACAGCACCAGCTCGCCCATCAACATCGTCGTGTCCTCGGC
CGGCCTGTCCCCGATCCGCGTGACCTCGCCCCCCACCGTGCAGTCCACCATCTCCTCCTCGCCCATCC
ACCAGCTGAGCTCCACCATCGGCACGTACGCCACCCTGTCGCCCACCAAGCGCCTGGTCCACGCGTCC
GAGCAGTACAGCAAGCACTCGCAGGAGCTGTATGCCACGGCCACCCTCCAGAGGCCGGGCAGCCTGGC
AGCTGGTTCCCGAGCCTCATACAGCAGCCAGCATGGGCACCTGGGCCCAGAGTTGCGGGCCCTGCAGT
CCCCAGAACACCACATAGATCCCATCTATGAAGACCGCGTCTATCAGAAGCCCCCTATGAGGAGTCTC
AGCCAGAGCCAGGGGGACCCTCTGCCGCCAGCACACACCGGCACCTACCGCACGAGCACAGCCCCATC
TTCCCCTGGTGTCGACTCCGTCCCCTTGCAGCGCACAGGCAGCCAGCACGGCCCACAGAATGCCGCCG
CGGCCACCTTCCAGAGGGCCAGCTATGCCGCCGGCCCAGCCTCCAATTACGCGGACCCCTACCGACAG
CTGCAGTATTGTCCCTCTGTTGAGTCTCCATACAGCAAATCCGGCCCTGCTCTCCCGCCTGAAGGCAC
CTTGGCCAGGTCCCCGTCCATTGATAGCATTCAGAAAGATCCCAGAGAATTTGGATGGAGAGACCCGG
AACTGCCGGAAGTGATTCAGATGTTGCAGCACCAGTTTCCCTCGGTCCAGTCTAACGCGGCAGCCTAC
TTGCAACACCTCTGTTTTGGAGACAACAAAATTAAAGCCGAGATAAGGAGACAAGGAGGCATCCAGCT
CCTGGTGGACCTGTTGGATCATCGGATGACCGAAGTCCACCGTAGTGCCTGTGGAGCTCTGAGAAACC
TGGTGTATGGGAAGGCCAACGATGATAACAAAATTGCCCTGAAAAACTGTGGTGGCATCCCAGCACTG
GTGAGGTTACTCCGCAAGACGACTGACCTGGAGATCCGGGAGCTGGTCACAGGAGTCCTTTGGAACCT
CTCCTCATGCGATGCACTCAAAATGCCAATCATCCAGGATGCCCTAGCAGTACTGACCAACGCGGTGA
TTATCCCCCACTCAGGCTGGGAAAATTCGCCTCTTCAGGATGATCGGAAAATACAGCTGCATTCATCA
CAGGTGCTGCGTAACGCCACCGGGTGCCTAAGGAATGTTAGTTCGGCCGGAGAGGAGGCCCGCAGAAG
GATGAGAGAGTGTGATGGGCTTACGGATGCCTTGCTGTACGTGATCCAGTCTGCGCTGGGGAGCAGTG
AGATCGATAGCAAGACCGTTGAAAACTGTGTGTGCATTTTAAGGAACCTCTCGTACCGGCTGGCGGCA
GAAACGTCTCAGGGACAGCACATGGGCACGGACGAGCTGGACGGGCTACTCTGTGGCGAGGCCAATGG
CAAGGATGCTGAGAGCTCTGGGTGCTGGGGCAAGAAGAAGAAGAAAAAGAAATCCCAAGATCAGTGGG
ATGGAGTAGGACCTCTTCCAGACTGTGCTGAACCACCAAAAGGGATCCAGATGCTGTGGCACCCATCA
ATAGTCAAACCCTACCTCACACTGCTCTCTGAGTGCTCAAATCCAGACACGCTGGAAGGGGCGGCAGG
CGCCCTGCAGAACTTGGCTGCAGGGAGCTGGAAGTGGTCAGTATATATCCGAGCCGCTGTCCGAAAAG
AGAAAGGCCTGCCCATCCTCGTGGAGCTGCTCCGAATAGACAATGACCGTGTGGTGTGCGCGGTGGCC
ACTGCGCTGCGGAACATGGCCTTGGACGTCAGAAATAAGGAGCTCATCGGCAAATACGCCATGCGAGA
CCTAGTCCACAGGCTTCCAGGAGGGAACAACAGCAACAACACTGCAAGCAAGGCCATGTCGGATGACA
CAGTGACAGCTGTCTGCTGCACACTGCACGAAGTGATTACCAAGAACATGGAGAACGCCAAGGCCTTA
CGGGATGCCGGTGGCATCGAGAAGTTGGTCGGCATCTCCAAAAGCAAAGGAGATAAACACTCTCCAAA
AGTGGTCAAGGCTGCATCTCAGGTCCTCAACAGCATGTGGCAGTACCGAGATCTGAGGAGTCTCTACA
AAAAGGATGGATGGTCACAATACCACTTTGTAGCCTCGTCTTCAACCATCGAGAGGGACCGGCAAAGG
CCCTACTCCTCCTCCCGCACGCCCTCCATCTCCCCTGTGCGCGTGTCTCCCAACAACCGCTCAGCAAG
TGCCCCAGCTTCACCTCGGGAAATGATCAGCCTCAAAGAAAGGAAAACAGACTACGAGTGCACCGGCA
GCAACGCCACCTACCACGGAGCTAAAGGCGAACACACTTCCAGGAAAGATGCCATGACAGCTCAAAAC
ACTGGAATTTCAACTTTGTATAGGAATTCTTATGGTGCGCCCGCTGAAGACATCAAACACAACCAGGT
TTCAGCACAGCCAGTCCCACAGGAGCCCAGCAGAAAAGATTACGAGACCTACCAGCCATTTCAGAATT
CCACAAGAAATTACGATGAGTCCTTCTTCGAGGACCAGGTCCACCATCGCCCTCCCGCCAGCGAGTAC
ACCATGCACCTGGGTCTCAAGTCCACCGGCAACTACGTTGACTTCTACTCAGCTGCCCGTCCCTACAG
TGAACTGAACTATGAAACGAGCCACTACCCGGCCTCCCCCGACTCCTGGGTGTGAGGAGCAGGGCACA
GGCGCTCCGGGAACAGTGCATGTGCATGCATACCACAAGACATTTCTTTCTGTTTTGTTTTTTTCTCC
TGCAAATTTAGTTTGTTAAAGCCTGTTCCATAGGAAGGCTGTGATAACCAGTAAGGAAATATTAAGAG
CTATTTTAGAAAGCTAAATGAATCGCAAGTTAACTTGGAAATCAGTAGAAAGCTAAAGTGATCCTAAA
TATGACAGTGGGCAGCACCTTTCTAGCGTGAGCTGTAGAGTAACGAGAAGTGCTTTATACTGAACGTG
GTTGATGGGAGGAGAGACGAGGCATTCGGGCCGGTGGGGCGTAAGGGTTATCGTTAAGCACAAGACAC
AGAATAGTTTACACACTGTGTGGGGGACGGCTTCTCACGCTTTGTTTACTCTCTTCATCCGTTGTGAC
TCTAGGCTTCAGGTTGCATTGGGGTTCCTCTGTACAGCAAGATGTTTCTTGCCTTTTGTTAATGCATT
GTTGTAAAGTATTTGATGTACATTACAGATTAAAGAAGAAAAGCGCGTTGTGTATATTACACCAATGC
CGCCGTGTTTCCTCATCTATGGTTCTAAATATTGCTTCAATTTCAAACTTTTGAAAGATGTATGGATT
TCCAGTTTTTCTTTACTTTCTCCCAGTATGTTTTAACAAAAAAAAAAAAAAGCAGGAAAAAAGGAATA
TTTAGCAGTATTGTTCGTTCTGATATGTGAATTTGTTTGTGACAACTAAACAAGGCATTCAGCAGTTT
CTGACAATTAACATACATCATTCCACACTCCTTGTCAACAAAGTGCTTTTTCACTGCCTAAAATTTTA
GATGTAGATATTTGAAATAGATTTTTTCATTTATACCAGTTTTCTTTATGATGATACAGTGTTAAAAG
AAAATAAATTACAATTGATCTGTCATCCATATTTGCTAAGAATGTCTATTTCCAAGAACCTATATCTT
ACAAAATACACATTCTTGCTTGTGTGTCTGTGTGTGTGGGTGTATGTATGTCCACGTACTAGAAAAAT
TACCTGTGTGAGATTTTATTTTGACAGAAGCTATTTCATTTGCTGTTCATCTTGTACAAACTTTGTTT
TTGGTTTTTTAGTATAAATGTACATCAGTTTGTTCCTTTTGTACTGTATCTATTCTTCTGACCATCTA
GTGACTCAGGATATTAGGCCAGTTGAAGTGGAGTTATAGGATTTTCACACATTCACGCATACTTGGAT
ATCAACCCTCTCTACAGGTCCGTTTCCTTACACATTTTATCCCCTCAGAACACGATAAACCATGGCCA
ATTCAGTTTCACTTTGGGGCCAATTACTTGAAGTATCCACAGAAAATGTAAACTGCTCGCCTTATTCC
CCCAAACTTTTTGTTCATGTGGCAGGGAGCAGTACACAACTATGGAATAGATTATTAAAACTGAACTT
GAAGCTAACGTTAGAATAGTGGTTAACAGAGCTAAGAAATAAGGCTAACCATCGAAAAGAACATGGGC
CGCGGGTAAACTAGCTTTGCTCTTTAGATGCAAGTGGCTGAGGCCAATAGGTGCCTAGTAAATGTTAA
CTGTTCTTGGAAAAAATAAAAACATCAGTAACAAAAGAAA
Variant 1 Peptide: (UniProt: Q9UQB3); CTND2_HUMAN Catenin delta-2
OS = Homo sapiens OX = 9606 GN = CTNND2 PE = 1 SV = 3.
SEQ ID NO: 21
MFARKPPGAAPLGAMPVPDQPSSASEKTSSLSPGLNTSNGDGSETETTSAILASVKEQELQFERLTRE
LEAERQIVASQLERCKLGSETGSMSSMSSAEEQFQWQSQDGQKDIEDELTTGLELVDSCIRSLQESGI
LDPQDYSTGERPSLLSQSALQLNSKPEGSFQYPASYHSNQTLALGETTPSQLPARGTQARATGQSFSQ
GTTSRAGHLAGPEPAPPPPPPPREPFAPSLGSAFHLPDAPPAAAAAALYYSSSTLPAPPRGGSPLAAP
QGGSPTKLQRGGSAPEGATYAAPRGSSPKQSPSRLAKSYSTSSPINIVVSSAGLSPIRVTSPPTVQST
ISSSPIHQLSSTIGTYATLSPTKRLVHASEQYSKHSQELYATATLQRPGSLAAGSRASYSSQHGHLGP
ELRALQSPEHHIDPIYEDRVYQKPPMRSLSQSQGDPLPPAHTGTYRTSTAPSSPGVDSVPLQRTGSQH
GPQNAAAATFQRASYAAGPASNYADPYRQLQYCPSVESPYSKSGPALPPEGTLARSPSIDSIQKDPRE
FGWRDPELPEVIQMLQHQFPSVQSNAAAYLQHLCFGDNKIKAEIRRQGGIQLLVDLLDHRMTEVHRSA
CGALRNLVYGKANDDNKIALKNCGGIPALVRLLRKTTDLEIRELVTGVLWNLSSCDALKMPIIQDALA
VLTNAVIIPHSGWENSPLQDDRKIQLHSSQVLRNATGCLRNVSSAGEEARRRMRECDGLTDALLYVIQ
SALGSSEIDSKTVENCVCILRNLSYRLAAETSQGQHMGTDELDGLLCGEANGKDAESSGCWGKKKKKK
KSQDQWDGVGPLPDCAEPPKGIQMLWHPSIVKPYLTLLSECSNPDTLEGAAGALQNLAAGSWKWSVYI
RAAVRKEKGLPILVELLRIDNDRVVCAVATALRNMALDVRNKELIGKYAMRDLVHRLPGGNNSNNTAS
KAMSDDTVTAVCCTLHEVITKNMENAKALRDAGGIEKLVGISKSKGDKHSPKVVKAASQVLNSMWQYR
DLRSLYKKDGWSQYHFVASSSTIERDRQRPYSSSRTPSISPVRVSPNNRSASAPASPREMISLKERKT
DYECTGSNATYHGAKGEHTSRKDAMTAQNTGISTLYRNSYGAPAEDIKHNQVSAQPVPQEPSRKDYET
YQPFQNSTRNYDESFFEDQVHHRPPASEYTMHLGLKSTGNYVDFYSAARPYSELNYETSHYPASPDSW
V
Homo sapiens catenin delta 2 (CTNND2), transcript variant 2 (Acc.
No.: NM_001288715.1).
SEQ ID NO: 22
GTTCCTTTTAGCCGTGGTGCAGGGAGGGAGGAGGGAGGTGGGTAGGATGCCAGGAACCGCCTGCTCCA
CAGCCTGGGGAAGGAGGGAAGAGTAACCCTTCATCTTTGGGACTAGTCGTCTTGTTTGCCTAGACTAC
AGTTTTCAAGTATTTAAGGACCATTTATTTCAAAAGGAATTACAGTTTGAAAGGCTGACCCGAGAGCT
GGAGGCTGAACGGCAGATCGTAGCCAGCCAGCTGGAGCGATGCAAGCTCGGATCCGAGACTGGCAGCA
TGAGCAGCATGAGTTCAGCAGAAGAGCAGTTTCAGTGGCAGTCACAAGATGGTCAAAAAGATATCGAA
GATGAGCTTACAACAGGTCTCGAGCTGGTGGACTCCTGTATTAGGTCACTACAGGAATCAGGAATACT
TGACCCACAGGATTATTCTACAGGTGAAAGGCCCAGCCTGCTCTCCCAGAGTGCACTTCAGCTCAATT
CCAAACCTGAAGGGTCTTTCCAGTATCCGGCCAGCTACCATAGCAACCAGACCCTGGCCCTGGGGGAA
ACCACCCCTTCACAGCTCCCGGCCCGAGGCACACAAGCCCGAGCTACGGGCCAGAGCTTCAGCCAGGG
CACGACCAGCCGCGCCGGCCACCTGGCGGGGCCCGAGCCCGCGCCGCCGCCGCCGCCGCCGCCGCGGG
AGCCGTTCGCGCCCAGCCTGGGCAGCGCCTTCCACCTGCCCGACGCGCCGCCCGCCGCCGCCGCCGCC
GCGCTCTACTACTCCAGCTCCACGCTGCCCGCGCCGCCGCGCGGGGGCTCCCCGCTGGCCGCGCCCCA
GGGCGGTTCGCCCACCAAGCTGCAGCGCGGCGGCTCGGCCCCCGAGGGCGCCACCTACGCCGCGCCGC
GCGGCTCCTCGCCCAAGCAGTCGCCCAGCCGCCTGGCCAAGTCCTACAGCACCAGCTCGCCCATCAAC
ATCGTCGTGTCCTCGGCCGGCCTGTCCCCGATCCGCGTGACCTCGCCCCCCACCGTGCAGTCCACCAT
CTCCTCCTCGCCCATCCACCAGCTGAGCTCCACCATCGGCACGTACGCCACCCTGTCGCCCACCAAGC
GCCTGGTCCACGCGTCCGAGCAGTACAGCAAGCACTCGCAGGAGCTGTATGCCACGGCCACCCTCCAG
AGGCCGGGCAGCCTGGCAGCTGGTTCCCGAGCCTCATACAGCAGCCAGCATGGGCACCTGGGCCCAGA
GTTGCGGGCCCTGCAGTCCCCAGAACACCACATAGATCCCATCTATGAAGACCGCGTCTATCAGAAGC
CCCCTATGAGGAGTCTCAGCCAGAGCCAGGGGGACCCTCTGCCGCCAGCACACACCGGCACCTACCGC
ACGAGCACAGCCCCATCTTCCCCTGGTGTCGACTCCGTCCCCTTGCAGCGCACAGGCAGCCAGCACGG
CCCACAGAATGCCGCCGCGGCCACCTTCCAGAGGGCCAGCTATGCCGCCGGCCCAGCCTCCAATTACG
CGGACCCCTACCGACAGCTGCAGTATTGTCCCTCTGTTGAGTCTCCATACAGCAAATCCGGCCCTGCT
CTCCCGCCTGAAGGCACCTTGGCCAGGTCCCCGTCCATTGATAGCATTCAGAAAGATCCCAGAGAATT
TGGATGGAGAGACCCGGAACTGCCGGAAGTGATTCAGATGTTGCAGCACCAGTTTCCCTCGGTCCAGT
CTAACGCGGCAGCCTACTTGCAACACCTCTGTTTTGGAGACAACAAAATTAAAGCCGAGATAAGGAGA
CAAGGAGGCATCCAGCTCCTGGTGGACCTGTTGGATCATCGGATGACCGAAGTCCACCGTAGTGCCTG
TGGAGCTCTGAGAAACCTGGTGTATGGGAAGGCCAACGATGATAACAAAATTGCCCTGAAAAACTGTG
GTGGCATCCCAGCACTGGTGAGGTTACTCCGCAAGACGACTGACCTGGAGATCCGGGAGCTGGTCACA
GGAGTCCTTTGGAACCTCTCCTCATGCGATGCACTCAAAATGCCAATCATCCAGGATGCCCTAGCAGT
ACTGACCAACGCGGTGATTATCCCCCACTCAGGCTGGGAAAATTCGCCTCTTCAGGATGATCGGAAAA
TACAGCTGCATTCATCACAGGTGCTGCGTAACGCCACCGGGTGCCTAAGGAATGTTAGTTCGGCCGGA
GAGGAGGCCCGCAGAAGGATGAGAGAGTGTGATGGGCTTACGGATGCCTTGCTGTACGTGATCCAGTC
TGCGCTGGGGAGCAGTGAGATCGATAGCAAGACCGTTGAAAACTGTGTGTGCATTTTAAGGAACCTCT
CGTACCGGCTGGCGGCAGAAACGTCTCAGGGACAGCACATGGGCACGGACGAGCTGGACGGGCTACTC
TGTGGCGAGGCCAATGGCAAGGATGCTGAGAGCTCTGGGTGCTGGGGCAAGAAGAAGAAGAAAAAGAA
ATCCCAAGATCAGTGGGATGGAGTAGGACCTCTTCCAGACTGTGCTGAACCACCAAAAGGGATCCAGA
TGCTGTGGCACCCATCAATAGTCAAACCCTACCTCACACTGCTCTCTGAGTGCTCAAATCCAGACACG
CTGGAAGGGGCGGCAGGCGCCCTGCAGAACTTGGCTGCAGGGAGCTGGAAGTGGTCAGTATATATCCG
AGCCGCTGTCCGAAAAGAGAAAGGCCTGCCCATCCTCGTGGAGCTGCTCCGAATAGACAATGACCGTG
TGGTGTGCGCGGTGGCCACTGCGCTGCGGAACATGGCCTTGGACGTCAGAAATAAGGAGCTCATCGGC
AAATACGCCATGCGAGACCTAGTCCACAGGCTTCCAGGAGGGAACAACAGCAACAACACTGCAAGCAA
GGCCATGTCGGATGACACAGTGACAGCTGTCTGCTGCACACTGCACGAAGTGATTACCAAGAACATGG
AGAACGCCAAGGCCTTACGGGATGCCGGTGGCATCGAGAAGTTGGTCGGCATCTCCAAAAGCAAAGGA
GATAAACACTCTCCAAAAGTGGTCAAGGCTGCATCTCAGGTCCTCAACAGCATGTGGCAGTACCGAGA
TCTGAGGAGTCTCTACAAAAAGGATGGATGGTCACAATACCACTTTGTAGCCTCGTCTTCAACCATCG
AGAGGGACCGGCAAAGGCCCTACTCCTCCTCCCGCACGCCCTCCATCTCCCCTGTGCGCGTGTCTCCC
AACAACCGCTCAGCAAGTGCCCCAGCTTCACCTCGGGAAATGATCAGCCTCAAAGAAAGGAAAACAGA
CTACGAGTGCACCGGCAGCAACGCCACCTACCACGGAGCTAAAGGCGAACACACTTCCAGGAAAGATG
CCATGACAGCTCAAAACACTGGAATTTCAACTTTGTATAGGAATTCTTATGGTGCGCCCGCTGAAGAC
ATCAAACACAACCAGGTTTCAGCACAGCCAGTCCCACAGGAGCCCAGCAGAAAAGATTACGAGACCTA
CCAGCCATTTCAGAATTCCACAAGAAATTACGATGAGTCCTTCTTCGAGGACCAGGTCCACCATCGCC
CTCCCGCCAGCGAGTACACCATGCACCTGGGTCTCAAGTCCACCGGCAACTACGTTGACTTCTACTCA
GCTGCCCGTCCCTACAGTGAACTGAACTATGAAACGAGCCACTACCCGGCCTCCCCCGACTCCTGGGT
GTGAGGAGCAGGGCACAGGCGCTCCGGGAACAGTGCATGTGCATGCATACCACAAGACATTTCTTTCT
GTTTTGTTTTTTTCTCCTGCAAATTTAGTTTGTTAAAGCCTGTTCCATAGGAAGGCTGTGATAACCAG
TAAGGAAATATTAAGAGCTATTTTAGAAAGCTAAATGAATCGCAAGTTAACTTGGAAATCAGTAGAAA
GCTAAAGTGATCCTAAATATGACAGTGGGCAGCACCTTTCTAGCGTGAGCTGTAGAGTAACGAGAAGT
GCTTTATACTGAACGTGGTTGATGGGAGGAGAGACGAGGCATTCGGGCCGGTGGGGCGTAAGGGTTAT
CGTTAAGCACAAGACACAGAATAGTTTACACACTGTGTGGGGGACGGCTTCTCACGCTTTGTTTACTC
TCTTCATCCGTTGTGACTCTAGGCTTCAGGTTGCATTGGGGTTCCTCTGTACAGCAAGATGTTTCTTG
CCTTTTGTTAATGCATTGTTGTAAAGTATTTGATGTACATTACAGATTAAAGAAGAAAAGCGCGTTGT
GTATATTACACCAATGCCGCCGTGTTTCCTCATCTATGGTTCTAAATATTGCTTCAATTTCAAACTTT
TGAAAGATGTATGGATTTCCAGTTTTTCTTTACTTTCTCCCAGTATGTTTTAACAAAAAAAAAAAAAA
GCAGGAAAAAAGGAATATTTAGCAGTATTGTTCGTTCTGATATGTGAATTTGTTTGTGACAACTAAAC
AAGGCATTCAGCAGTTTCTGACAATTAACATACATCATTCCACACTCCTTGTCAACAAAGTGCTTTTT
CACTGCCTAAAATTTTAGATGTAGATATTTGAAATAGATTTTTTCATTTATACCAGTTTTCTTTATGA
TGATACAGTGTTAAAAGAAAATAAATTACAATTGATCTGTCATCCATATTTGCTAAGAATGTCTATTT
CCAAGAACCTATATCTTACAAAATACACATTCTTGCTTGTGTGTCTGTGTGTGTGGGTGTATGTATGT
CCACGTACTAGAAAAATTACCTGTGTGAGATTTTATTTTGACAGAAGCTATTTCATTTGCTGTTCATC
TTGTACAAACTTTGTTTTTGGTTTTTTAGTATAAATGTACATCAGTTTGTTCCTTTTGTACTGTATCT
ATTCTTCTGACCATCTAGTGACTCAGGATATTAGGCCAGTTGAAGTGGAGTTATAGGATTTTCACACA
TTCACGCATACTTGGATATCAACCCTCTCTACAGGTCCGTTTCCTTACACATTTTATCCCCTCAGAAC
ACGATAAACCATGGCCAATTCAGTTTCACTTTGGGGCCAATTACTTGAAGTATCCACAGAAAATGTAA
ACTGCTCGCCTTATTCCCCCAAACTTTTTGTTCATGTGGCAGGGAGCAGTACACAACTATGGAATAGA
TTATTAAAACTGAACTTGAAGCTAACGTTAGAATAGTGGTTAACAGAGCTAAGAAATAAGGCTAACCA
TCGAAAAGAACATGGGCCGCGGGTAAACTAGCTTTGCTCTTTAGATGCAAGTGGCTGAGGCCAATAGG
TGCCTAGTAAATGTTAACTGTTCTTGGAAAAAATAAAAACATCAGTAACAAAA
Variant 2 Peptide from NM_001288715.1 Homo sapiens catenin delta 2
(CTNND2), transcript variant 2..
SEQ ID NO: 23
MSSMSSAEEQFQWQSQDGQKDIEDELTTGLELVDSCIRSLQESGILDPQDYSTGERPSLLSQSALQLN
SKPEGSFQYPASYHSNQTLALGETTPSQLPARGTQARATGQSFSQGTTSRAGHLAGPEPAPPPPPPPR
EPFAPSLGSAFHLPDAPPAAAAAALYYSSSTLPAPPRGGSPLAAPQGGSPTKLQRGGSAPEGATYAAP
RGSSPKQSPSRLAKSYSTSSPINIVVSSAGLSPIRVTSPPTVQSTISSSPIHQLSSTIGTYATLSPTK
RLVHASEQYSKHSQELYATATLQRPGSLAAGSRASYSSQHGHLGPELRALQSPEHHIDPIYEDRVYQK
PPMRSLSQSQGDPLPPAHTGTYRTSTAPSSPGVDSVPLQRTGSQHGPQNAAAATFQRASYAAGPASNY
ADPYRQLQYCPSVESPYSKSGPALPPEGTLARSPSIDSIQKDPREFGWRDPELPEVIQMLQHQFPSVQ
SNAAAYLQHLCFGDNKIKAEIRRQGGIQLLVDLLDHRMTEVHRSACGALRNLVYGKANDDNKIALKNC
GGIPALVRLLRKTTDLEIRELVTGVLWNLSSCDALKMPIIQDALAVLTNAVIIPHSGWENSPLQDDRK
IQLHSSQVLRNATGCLRNVSSAGEEARRRMRECDGLTDALLYVIQSALGSSEIDSKTVENCVCILRNL
SYRLAAETSQGQHMGTDELDGLLCGEANGKDAESSGCWGKKKKKKKSQDQWDGVGPLPDCAEPPKGIQ
MLWHPSIVKPYLTLLSECSNPDTLEGAAGALQNLAAGSWKWSVYIRAAVRKEKGLPILVELLRIDNDR
VVCAVATALRNMALDVRNKELIGKYAMRDLVHRLPGGNNSNNTASKAMSDDTVTAVCCTLHEVITKNM
ENAKALRDAGGIEKLVGISKSKGDKHSPKVVKAASQVLNSMWQYRDLRSLYKKDGWSQYHFVASSSTI
ERDRQRPYSSSRTPSISPVRVSPNNRSASAPASPREMISLKERKTDYECTGSNATYHGAKGEHTSRKD
AMTAQNTGISTLYRNSYGAPAEDIKHNQVSAQPVPQEPSRKDYETYQPFQNSTRNYDESFFEDQVHHR
PPASEYTMHLGLKSTGNYVDFYSAARPYSELNYETSHYPASPDSWV
Homo sapiens catenin delta 2 (CTNND2), transcript variant 3 (Acc.
No.: NM_001288716.1); Transcript length: 4551; CDS length: 2667; CDS
start: 272; CDS end: 2938; peptide length: 888.
SEQ ID NO: 24
GTTCCTTTTAGCCGTGGTGCAGGGAGGGAGGAGGGAGGTGGGTAGGATGCCAGGAACCGCCTGCTCCA
CAGCCTGGGGAAGGAGGGAAGAGTAACCCTTCATCTTTGGGACTAGTCGTCTTGTTTGCCTAGACTAC
AGTTTTCAAGTATTTAAGGACCATTTATTTCAAAAGGAATTACAGTTTGAAAGGCTGACCCGAGAGCT
GGAGGCTGAACGGCAGATCGTAGCCAGCCAGCTGGAGCGATGCAAGCTCGGATCCGAGACTGGCAGCA
TGAGCAGCATGAGTTCAGCAGAAGAGCAGTTTCAGTGGCAGTCACAAGATGGTCAAAAAGATATCGAA
GATGAGCTTACAACAGGTCTCGAGCTGGTGGACTCCTGTATTAGGTCACTACAGGAATCAGGAATACT
TGACCCACAGGATTATTCTACAGGTGAAACTGGTTCCCGAGCCTCATACAGCAGCCAGCATGGGCACC
TGGGCCCAGAGTTGCGGGCCCTGCAGTCCCCAGAACACCACATAGATCCCATCTATGAAGACCGCGTC
TATCAGAAGCCCCCTATGAGGAGTCTCAGCCAGAGCCAGGGGGACCCTCTGCCGCCAGCACACACCGG
CACCTACCGCACGAGCACAGCCCCATCTTCCCCTGGTGTCGACTCCGTCCCCTTGCAGCGCACAGGCA
GCCAGCACGGCCCACAGAATGCCGCCGCGGCCACCTTCCAGAGGGCCAGCTATGCCGCCGGCCCAGCC
TCCAATTACGCGGACCCCTACCGACAGCTGCAGTATTGTCCCTCTGTTGAGTCTCCATACAGCAAATC
CGGCCCTGCTCTCCCGCCTGAAGGCACCTTGGCCAGGTCCCCGTCCATTGATAGCATTCAGAAAGATC
CCAGAGAATTTGGATGGAGAGACCCGGAACTGCCGGAAGTGATTCAGATGTTGCAGCACCAGTTTCCC
TCGGTCCAGTCTAACGCGGCAGCCTACTTGCAACACCTCTGTTTTGGAGACAACAAAATTAAAGCCGA
GATAAGGAGACAAGGAGGCATCCAGCTCCTGGTGGACCTGTTGGATCATCGGATGACCGAAGTCCACC
GTAGTGCCTGTGGAGCTCTGAGAAACCTGGTGTATGGGAAGGCCAACGATGATAACAAAATTGCCCTG
AAAAACTGTGGTGGCATCCCAGCACTGGTGAGGTTACTCCGCAAGACGACTGACCTGGAGATCCGGGA
GCTGGTCACAGGAGTCCTTTGGAACCTCTCCTCATGCGATGCACTCAAAATGCCAATCATCCAGGATG
CCCTAGCAGTACTGACCAACGCGGTGATTATCCCCCACTCAGGCTGGGAAAATTCGCCTCTTCAGGAT
GATCGGAAAATACAGCTGCATTCATCACAGGTGCTGCGTAACGCCACCGGGTGCCTAAGGAATGTTAG
TTCGGCCGGAGAGGAGGCCCGCAGAAGGATGAGAGAGTGTGATGGGCTTACGGATGCCTTGCTGTACG
TGATCCAGTCTGCGCTGGGGAGCAGTGAGATCGATAGCAAGACCGTTGAAAACTGTGTGTGCATTTTA
AGGAACCTCTCGTACCGGCTGGCGGCAGAAACGTCTCAGGGACAGCACATGGGCACGGACGAGCTGGA
CGGGCTACTCTGTGGCGAGGCCAATGGCAAGGATGCTGAGAGCTCTGGGTGCTGGGGCAAGAAGAAGA
AGAAAAAGAAATCCCAAGATCAGTGGGATGGAGTAGGACCTCTTCCAGACTGTGCTGAACCACCAAAA
GGGATCCAGATGCTGTGGCACCCATCAATAGTCAAACCCTACCTCACACTGCTCTCTGAGTGCTCAAA
TCCAGACACGCTGGAAGGGGCGGCAGGCGCCCTGCAGAACTTGGCTGCAGGGAGCTGGAAGTGGTCAG
TATATATCCGAGCCGCTGTCCGAAAAGAGAAAGGCCTGCCCATCCTCGTGGAGCTGCTCCGAATAGAC
AATGACCGTGTGGTGTGCGCGGTGGCCACTGCGCTGCGGAACATGGCCTTGGACGTCAGAAATAAGGA
GCTCATCGGCAAATACGCCATGCGAGACCTAGTCCACAGGCTTCCAGGAGGGAACAACAGCAACAACA
CTGCAAGCAAGGCCATGTCGGATGACACAGTGACAGCTGTCTGCTGCACACTGCACGAAGTGATTACC
AAGAACATGGAGAACGCCAAGGCCTTACGGGATGCCGGTGGCATCGAGAAGTTGGTCGGCATCTCCAA
AAGCAAAGGAGATAAACACTCTCCAAAAGTGGTCAAGGCTGCATCTCAGGTCCTCAACAGCATGTGGC
AGTACCGAGATCTGAGGAGTCTCTACAAAAAGGATGGATGGTCACAATACCACTTTGTAGCCTCGTCT
TCAACCATCGAGAGGGACCGGCAAAGGCCCTACTCCTCCTCCCGCACGCCCTCCATCTCCCCTGTGCG
CGTGTCTCCCAACAACCGCTCAGCAAGTGCCCCAGCTTCACCTCGGGAAATGATCAGCCTCAAAGAAA
GGAAAACAGACTACGAGTGCACCGGCAGCAACGCCACCTACCACGGAGCTAAAGGCGAACACACTTCC
AGGAAAGATGCCATGACAGCTCAAAACACTGGAATTTCAACTTTGTATAGGAATTCTTATGGTGCGCC
CGCTGAAGACATCAAACACAACCAGGTTTCAGCACAGCCAGTCCCACAGGAGCCCAGCAGAAAAGATT
ACGAGACCTACCAGCCATTTCAGAATTCCACAAGAAATTACGATGAGTCCTTCTTCGAGGACCAGGTC
CACCATCGCCCTCCCGCCAGCGAGTACACCATGCACCTGGGTCTCAAGTCCACCGGCAACTACGTTGA
CTTCTACTCAGCTGCCCGTCCCTACAGTGAACTGAACTATGAAACGAGCCACTACCCGGCCTCCCCCG
ACTCCTGGGTGTGAGGAGCAGGGCACAGGCGCTCCGGGAACAGTGCATGTGCATGCATACCACAAGAC
ATTTCTTTCTGTTTTGTTTTTTTCTCCTGCAAATTTAGTTTGTTAAAGCCTGTTCCATAGGAAGGCTG
TGATAACCAGTAAGGAAATATTAAGAGCTATTTTAGAAAGCTAAATGAATCGCAAGTTAACTTGGAAA
TCAGTAGAAAGCTAAAGTGATCCTAAATATGACAGTGGGCAGCACCTTTCTAGCGTGAGCTGTAGAGT
AACGAGAAGTGCTTTATACTGAACGTGGTTGATGGGAGGAGAGACGAGGCATTCGGGCCGGTGGGGCG
TAAGGGTTATCGTTAAGCACAAGACACAGAATAGTTTACACACTGTGTGGGGGACGGCTTCTCACGCT
TTGTTTACTCTCTTCATCCGTTGTGACTCTAGGCTTCAGGTTGCATTGGGGTTCCTCTGTACAGCAAG
ATGTTTCTTGCCTTTTGTTAATGCATTGTTGTAAAGTATTTGATGTACATTACAGATTAAAGAAGAAA
AGCGCGTTGTGTATATTACACCAATGCCGCCGTGTTTCCTCATCTATGGTTCTAAATATTGCTTCAAT
TTCAAACTTTTGAAAGATGTATGGATTTCCAGTTTTTCTTTACTTTCTCCCAGTATGTTTTAACAAAA
AAAAAAAAAAGCAGGAAAAAAGGAATATTTAGCAGTATTGTTCGTTCTGATATGTGAATTTGTTTGTG
ACAACTAAACAAGGCATTCAGCAGTTTCTGACAATTAACATACATCATTCCACACTCCTTGTCAACAA
AGTGCTTTTTCACTGCCTAAAATTTTAGATGTAGATATTTGAAATAGATTTTTTCATTTATACCAGTT
TTCTTTATGATGATACAGTGTTAAAAGAAAATAAATTACAATTGATCTGTCATCCATATTTGCTAAGA
ATGTCTATTTCCAAGAACCTATATCTTACAAAATACACATTCTTGCTTGTGTGTCTGTGTGTGTGGGT
GTATGTATGTCCACGTACTAGAAAAATTACCTGTGTGAGATTTTATTTTGACAGAAGCTATTTCATTT
GCTGTTCATCTTGTACAAACTTTGTTTTTGGTTTTTTAGTATAAATGTACATCAGTTTGTTCCTTTTG
TACTGTATCTATTCTTCTGACCATCTAGTGACTCAGGATATTAGGCCAGTTGAAGTGGAGTTATAGGA
TTTTCACACATTCACGCATACTTGGATATCAACCCTCTCTACAGGTCCGTTTCCTTACACATTTTATC
CCCTCAGAACACGATAAACCATGGCCAATTCAGTTTCACTTTGGGGCCAATTACTTGAAGTATCCACA
GAAAATGTAAACTGCTCGCCTTATTCCCCCAAACTTTTTGTTCATGTGGCAGGGAGCAGTACACAACT
ATGGAATAGATTATTAAAACTGAACTTGAAGCTAACGTTAGAATAGTGGTTAACAGAGCTAAGAAATA
AGGCTAACCATCGAAAAGAACATGGGCCGCGGGTAAACTAGCTTTGCTCTTTAGATGCAAGTGGCTGA
GGCCAATAGGTGCCTAGTAAATGTTAACTGTTCTTGGAAAAAATAAAAACATCAGTAACAAAA
Variant 3 Peptide from NM_001288716.1 Homo sapiens catenin delta 2
(CTNND2), transcript variant 3.
SEQ ID NO: 25
MSSMSSAEEQFQWQSQDGQKDIEDELTTGLELVDSCIRSLQESGILDPQDYSTGETGSRASYSSQHGH
LGPELRALQSPEHHIDPIYEDRVYQKPPMRSLSQSQGDPLPPAHTGTYRTSTAPSSPGVDSVPLQRTG
SQHGPQNAAAATFQRASYAAGPASNYADPYRQLQYCPSVESPYSKSGPALPPEGTLARSPSIDSIQKD
PREFGWRDPELPEVIQMLQHQFPSVQSNAAAYLQHLCFGDNKIKAEIRRQGGIQLLVDLLDHRMTEVH
RSACGALRNLVYGKANDDNKIALKNCGGIPALVRLLRKTTDLEIRELVTGVLWNLSSCDALKMPIIQD
ALAVLTNAVIIPHSGWENSPLQDDRKIQLHSSQVLRNATGCLRNVSSAGEEARRRMRECDGLTDALLY
VIQSALGSSEIDSKTVENCVCILRNLSYRLAAETSQGQHMGTDELDGLLCGEANGKDAESSGCWGKKK
KKKKSQDQWDGVGPLPDCAEPPKGIQMLWHPSIVKPYLTLLSECSNPDTLEGAAGALQNLAAGSWKWS
VYIRAAVRKEKGLPILVELLRIDNDRVVCAVATALRNMALDVRNKELIGKYAMRDLVHRLPGGNNSNN
TASKAMSDDTVTAVCCTLHEVITKNMENAKALRDAGGIEKLVGISKSKGDKHSPKVVKAASQVLNSMW
QYRDLRSLYKKDGWSQYHFVASSSTIERDRQRPYSSSRTPSISPVRVSPNNRSASAPASPREMISLKE
RKTDYECTGSNATYHGAKGEHTSRKDAMTAQNTGISTLYRNSYGAPAEDIKHNQVSAQPVPQEPSRKD
YETYQPFQNSTRNYDESFFEDQVHHRPPASEYTMHLGLKSTGNYVDFYSAARPYSELNYETSHYPASP
DSWV
Homo sapiens catenin delta 2 (CTNND2), transcript variant 4 (Acc.
No.: NM_001288717.1); Transcript length: 4916; CDS length: 2379; CDS
start: 925; CDS end: 3303; peptide length: 792.
SEQ ID NO: 26
GCACCCGGGGCCGGTTTCGGCGGCGGGGCGGACGCTCCCTCACTAGCCGGGCGCCCGGGCCGGCCGCC
GAGGTTTAGGTGGAGGCTGAGGCTGCCGCGCGCCGCGGGAGGAGCCTCGCCCTCGGCGGCTGAGGCGG
CGGCGGCACAGGTGGCGCGGGCCGCGCGCGGGGCGCAGCTCGGGAGCGCTCGGCGCCGGGCGCCGCGG
CGGCCCCAGGCTCGCGCCCGCGGCGACAGCCGGCTGAGCGGAGCTGCGGGCGCGGCGGCTGCTCTCTC
AGCCCGGCCGGCTGGCGGCGGCGCCCGGAGCCCCGAGGAGCCCGCAGGAGCCCGTGGAGCCCGCGGAG
CCGGGCAGGGGGCGCTGCGCTCGCCGAGCTCGGAGGGGCCGCCGGGCCGGGCGCTGCGCACTCGCGTC
GGGAGCCGCCTCTCGCCCGCGGCGCTCGCCCCTGCCGCCCGCCAGCATCCCTTGTCCCGCGGCCGCGC
TCAGACAACAAAAGCGGAAGATGCTGCAGTTGGGCAAGGTCAGGACCTTGCCCTGAAGCCGGGCGGCG
CCGCGCACGCCTCTTCCCGGACTGAGGAGCTGTCGCCGGCGGAGGGTGCATGTTTGCGAGGAAGCCGC
CGGGCGCCGCGCCTTTGGGAGCTATGCCTGTTCCAGACCAGCCTTCATCAGCCTCAGAGAAGACGAGT
TCCCTGAGCCCCGGCTTAAACACCTCCAACGGGGATGGCTCTGAAACAGAAACCACCTCTGCCATCCT
CGCCTCAGTCAAAGAACAGGAATTACAGTTTGAAAGGCTGACCCGAGAGCTGGAGGCTGAACGGCAGA
TCGTAGCCAGCCAGCTGGAGCGATGCAAGCTCGGATCCGAGACTGGCAGCATGAGCAGCATGAGTTCA
GCAGAAGAGCAGTTTCAGTGGCAGTCACAAGATGGTCAAAAAGATATCGAAGATGAGCTTACAACAGG
TCTCGAGCTGGTGGACTCCTGTATTAGGTCACTACAGGAATCAGGAATACTTGACCCACAGGATTATT
CTACAGGTGAAAGGCCCAGCCTGCTCTCCCAGAGTGCACTTCAGCTCAATTCCAAACCTGAAGGGTCT
TTCCAGTATCCGGCCAGCTACCATAGCAACCAGACCCTGGCCCTGGGGGAAACCACCCCTTCACAGCT
CCCGGCCCGAGGCACACAAGCCCGAGCTACGGGCCAGAGCTTCAGCCAGCTGGTTCCCGAGCCTCATA
CAGCAGCCAGCATGGGCACCTGGGCCCAGAGTTGCGGGCCCTGCAGTCCCCAGAACACCACATAGATC
CCATCTATGAAGACCGCGTCTATCAGAAGCCCCCTATGAGGAGTCTCAGCCAGAGCCAGGGGGACCCT
CTGCCGCCAGCACACACCGGCACCTACCGCACGAGCACAGCCCCATCTTCCCCTGGTGTCGACTCCGT
CCCCTTGCAGCGCACAGGCAGCCAGCACGGCCCACAGAATGCCGCCGCGGCCACCTTCCAGAGGGCCA
GCTATGCCGCCGGCCCAGCCTCCAATTACGCGGACCCCTACCGACAGCTGCAGTATTGTCCCTCTGTT
GAGTCTCCATACAGCAAATCCGGCCCTGCTCTCCCGCCTGAAGGCACCTTGGCCAGGTCCCCGTCCAT
TGATAGCATTCAGAAAGATCCCAGAGAATTTGGATGGAGAGACCCGGAACTGCCGGAAGTGATTCAGA
TGTTGCAGCACCAGTTTCCCTCGGTCCAGTCTAACGCGGCAGCCTACTTGCAACACCTCTGTTTTGGA
GACAACAAAATTAAAGCCGAGATAAGGAGACAAGGAGGCATCCAGCTCCTGGTGGACCTGTTGGATCA
TCGGATGACCGAAGTCCACCGTAGTGCCTGTGGAGCTCTGAGAAACCTGGTGTATGGGAAGGCCAACG
ATGATAACAAAATTGCCCTGAAAAACTGTGGTGGCATCCCAGCACTGGTGAGGTTACTCCGCAAGACG
ACTGACCTGGAGATCCGGGAGCTGGTCACAGGAGTCCTTTGGAACCTCTCCTCATGCGATGCACTCAA
AATGCCAATCATCCAGGATGCCCTAGCAGTACTGACCAACGCGGTGATTATCCCCCACTCAGGCTGGG
AAAATTCGCCTCTTCAGGATGATCGGAAAATACAGCTGCATTCATCACAGGTGCTGCGTAACGCCACC
GGGTGCCTAAGGAATGTTAGTTCGGCCGGAGAGGAGGCCCGCAGAAGGATGAGAGAGTGTGATGGGCT
TACGGATGCCTTGCTGTACGTGATCCAGTCTGCGCTGGGGAGCAGTGAGATCGATAGCAAGACCGTTG
AAAACTGTGTGTGCATTTTAAGGAACCTCTCGTACCGGCTGGCGGCAGAAACGTCTCAGGGACAGCAC
ATGGGCACGGACGAGCTGGACGGGCTACTCTGTGGCGAGGCCAATGGCAAGGATGCTGAGAGCTCTGG
GTGCTGGGGCAAGAAGAAGAAGAAAAAGAAATCCCAAGATCAGTGGGATGGAGTAGGACCTCTTCCAG
ACTGTGCTGAACCACCAAAAGGGATCCAGATGCTGTGGCACCCATCAATAGTCAAACCCTACCTCACA
CTGCTCTCTGAGTGCTCAAATCCAGACACGCTGGAAGGGGCGGCAGGCGCCCTGCAGAACTTGGCTGC
AGGGAGCTGGAAGTGGTCAGTATATATCCGAGCCGCTGTCCGAAAAGAGAAAGGCCTGCCCATCCTCG
TGGAGCTGCTCCGAATAGACAATGACCGTGTGGTGTGCGCGGTGGCCACTGCGCTGCGGAACATGGCC
TTGGACGTCAGAAATAAGGAGCTCATCGGCAAATACGCCATGCGAGACCTAGTCCACAGGCTTCCAGG
AGGGAACAACAGCAACAACACTGCAAGCAAGGCCATGTCGGATGACACAGTGACAGCTGTCTGCTGCA
CACTGCACGAAGTGATTACCAAGAACATGGAGAACGCCAAGGCCTTACGGGATGCCGGTGGCATCGAG
AAGTTGGTCGGCATCTCCAAAAGCAAAGGAGATAAACACTCTCCAAAAGTGGTCAAGGCTGCATCTCA
GGTCCTCAACAGCATGTGGCAGTACCGAGATCTGAGGAGTCTCTACAAAAAGGATGGATGGTCACAAT
ACCACTTTGTAGCCTCGTCTTCAACCATCGAGAGGGACCGGCAAAGGCCCTACTCCTCCTCCCGCACG
CCCTCCATCTCCCCTGTGCGCGTGTCTCCCAACAACCGCTCAGCAAGTGCCCCAGCTTCACCTCGGGA
AATGATCAGCCTCAAAGAAAGGAAAACAGACTACGAGTGCACCGGCAGCAACGCCACCTACCACGGAG
CTAAAGGCGAACACACTTCCAGGAAAGATGCCATGACAGCTCAAAACACTGGAATTTCAACTTTGTAT
AGGAATTCTTATGGTGCGCCCGCTGAAGACATCAAACACAACCAGGTTTCAGCACAGCCAGTCCCACA
GGAGCCCAGCAGAAAAGATTACGAGACCTACCAGCCATTTCAGAATTCCACAAGAAATTACGATGAGT
CCTTCTTCGAGGACCAGGTCCACCATCGCCCTCCCGCCAGCGAGTACACCATGCACCTGGGTCTCAAG
TCCACCGGCAACTACGTTGACTTCTACTCAGCTGCCCGTCCCTACAGTGAACTGAACTATGAAACGAG
CCACTACCCGGCCTCCCCCGACTCCTGGGTGTGAGGAGCAGGGCACAGGCGCTCCGGGAACAGTGCAT
GTGCATGCATACCACAAGACATTTCTTTCTGTTTTGTTTTTTTCTCCTGCAAATTTAGTTTGTTAAAG
CCTGTTCCATAGGAAGGCTGTGATAACCAGTAAGGAAATATTAAGAGCTATTTTAGAAAGCTAAATGA
ATCGCAAGTTAACTTGGAAATCAGTAGAAAGCTAAAGTGATCCTAAATATGACAGTGGGCAGCACCTT
TCTAGCGTGAGCTGTAGAGTAACGAGAAGTGCTTTATACTGAACGTGGTTGATGGGAGGAGAGACGAG
GCATTCGGGCCGGTGGGGCGTAAGGGTTATCGTTAAGCACAAGACACAGAATAGTTTACACACTGTGT
GGGGGACGGCTTCTCACGCTTTGTTTACTCTCTTCATCCGTTGTGACTCTAGGCTTCAGGTTGCATTG
GGGTTCCTCTGTACAGCAAGATGTTTCTTGCCTTTTGTTAATGCATTGTTGTAAAGTATTTGATGTAC
ATTACAGATTAAAGAAGAAAAGCGCGTTGTGTATATTACACCAATGCCGCCGTGTTTCCTCATCTATG
GTTCTAAATATTGCTTCAATTTCAAACTTTTGAAAGATGTATGGATTTCCAGTTTTTCTTTACTTTCT
CCCAGTATGTTTTAACAAAAAAAAAAAAAAGCAGGAAAAAAGGAATATTTAGCAGTATTGTTCGTTCT
GATATGTGAATTTGTTTGTGACAACTAAACAAGGCATTCAGCAGTTTCTGACAATTAACATACATCAT
TCCACACTCCTTGTCAACAAAGTGCTTTTTCACTGCCTAAAATTTTAGATGTAGATATTTGAAATAGA
TTTTTTCATTTATACCAGTTTTCTTTATGATGATACAGTGTTAAAAGAAAATAAATTACAATTGATCT
GTCATCCATATTTGCTAAGAATGTCTATTTCCAAGAACCTATATCTTACAAAATACACATTCTTGCTT
GTGTGTCTGTGTGTGTGGGTGTATGTATGTCCACGTACTAGAAAAATTACCTGTGTGAGATTTTATTT
TGACAGAAGCTATTTCATTTGCTGTTCATCTTGTACAAACTTTGTTTTTGGTTTTTTAGTATAAATGT
ACATCAGTTTGTTCCTTTTGTACTGTATCTATTCTTCTGACCATCTAGTGACTCAGGATATTAGGCCA
GTTGAAGTGGAGTTATAGGATTTTCACACATTCACGCATACTTGGATATCAACCCTCTCTACAGGTCC
GTTTCCTTACACATTTTATCCCCTCAGAACACGATAAACCATGGCCAATTCAGTTTCACTTTGGGGCC
AATTACTTGAAGTATCCACAGAAAATGTAAACTGCTCGCCTTATTCCCCCAAACTTTTTGTTCATGTG
GCAGGGAGCAGTACACAACTATGGAATAGATTATTAAAACTGAACTTGAAGCTAACGTTAGAATAGTG
GTTAACAGAGCTAAGAAATAAGGCTAACCATCGAAAAGAACATGGGCCGCGGGTAAACTAGCTTTGCT
CTTTAGATGCAAGTGGCTGAGGCCAATAGGTGCCTAGTAAATGTTAACTGTTCTTGGAAAAAATAAAA
ACATCAGTAACAAAAGAAA
Variant 4 Peptide from NM_001288717.2 Homo sapiens catenin delta 2
(CTNND2), transcript variant 4..
SEQ ID NO: 27
MRSLSQSQGDPLPPAHTGTYRTSTAPSSPGVDSVPLQRTGSQHGPQNAAAATFQRASYAAGPASNYAD
PYRQLQYCPSVESPYSKSGPALPPEGTLARSPSIDSIQKDPREFGWRDPELPEVIQMLQHQFPSVQSN
AAAYLQHLCFGDNKIKAEIRRQGGIQLLVDLLDHRMTEVHRSACGALRNLVYGKANDDNKIALKNCGG
IPALVRLLRKTTDLEIRELVTGVLWNLSSCDALKMPIIQDALAVLTNAVIIPHSGWENSPLQDDRKIQ
LHSSQVLRNATGCLRNVSSAGEEARRRMRECDGLTDALLYVIQSALGSSEIDSKTVENCVCILRNLSY
RLAAETSQGQHMGTDELDGLLCGEANGKDAESSGCWGKKKKKKKSQDQWDGVGPLPDCAEPPKGIQML
WHPSIVKPYLTLLSECSNPDTLEGAAGALQNLAAGSWKWSVYIRAAVRKEKGLPILVELLRIDNDRVV
CAVATALRNMALDVRNKELIGKYAMRDLVHRLPGGNNSNNTASKAMSDDTVTAVCCTLHEVITKNMEN
AKALRDAGGIEKLVGISKSKGDKHSPKVVKAASQVLNSMWQYRDLRSLYKKDGWSQYHFVASSSTIER
DRQRPYSSSRTPSISPVRVSPNNRSASAPASPREMISLKERKTDYECTGSNATYHGAKGEHTSRKDAM
TAQNTGISTLYRNSYGAPAEDIKHNQVSAQPVPQEPSRKDYETYQPFQNSTRNYDESFFEDQVHHRPP
ASEYTMHLGLKSTGNYVDFYSAARPYSELNYETSHYPASPDSWV
Homo sapiens catenin delta 2 (CTNND2), transcript variant 6 (Acc.
No.: NM_001364128.1); Transcript length: 4772; CDS length: 2742; CDS
start: 432; CDS end: 3173; peptide length: 913.
SEQ ID NO: 28
ACTGGGCTTGTGGTATCTCTGATATGTCTTGCTTTGCTTTCTTTTAAACTGTTTGCGAGTGAAAGCAG
AAATTACCATAGGATAATTCAGCGTCCTGACTTTTTCGAGGCTGTTCTTCACTCCCTTCTCGGTTTTT
GAGCTGCTCTGTGGTTCCTTTTAGCCGTGGTGCAGGGAGGGAGGAGGGAGGTGGGTAGGATGCCAGGA
ACCGCCTGCTCCACAGCCTGGGGAAGGAGGGAAGAGTAACCCTTCATCTTTGGGACTAGTCGTCTTGT
TTGCCTAGACTACAGTTTTCAAGTATTTAAGGACCATTTATTTCAAAAGGTACAGTAAAGGAATTACA
GTTTGAAAGGCTGACCCGAGAGCTGGAGGCTGAACGGCAGATCGTAGCCAGCCAGCTGGAGCGATGCA
AGCTCGGATCCGAGACTGGCAGCATGAGCAGCATGAGTTCAGCAGAAGAGCAGTTTCAGTGGCAGTCA
CAAGATGGTCAAAAAGATATCGAAGATGAGCTTACAACAGGTCTCGAGCTGGTGGACTCCTGTATTAG
GTCACTACAGGAATCAGGAATACTTGACCCACAGGATTATTCTACAGGTGAAACTGGTTCCCGAGCCT
CATACAGCAGCCAGCATGGGCACCTGGGCCCAGAGTTGCGGGCCCTGCAGTCCCCAGAACACCACATA
GATCCCATCTATGAAGACCGCGTCTATCAGAAGCCCCCTATGAGGAGTCTCAGCCAGAGCCAGGGGGA
CCCTCTGCCGCCAGCACACACCGGCACCTACCGCACGAGCACAGCCCCATCTTCCCCTGGTGTCGACT
CCGTCCCCTTGCAGCGCACAGGCAGCCAGCACGGCCCACAGAATGCCGCCGCGGCCACCTTCCAGAGG
GCCAGCTATGCCGCCGGCCCAGCCTCCAATTACGCGGACCCCTACCGACAGCTGCAGTATTGTCCCTC
TGTTGAGTCTCCATACAGCAAATCCGGCCCTGCTCTCCCGCCTGAAGGCACCTTGGCCAGGTCCCCGT
CCATTGATAGCATTCAGAAAGATCCCAGAGAATTTGGATGGAGAGACCCGGAACTGCCGGAAGTGATT
CAGATGTTGCAGCACCAGTTTCCCTCGGTCCAGTCTAACGCGGCAGCCTACTTGCAACACCTCTGTTT
TGGAGACAACAAAATTAAAGCCGAGATAAGGAGACAAGGAGGCATCCAGCTCCTGGTGGACCTGTTGG
ATCATCGGATGACCGAAGTCCACCGTAGTGCCTGTGGAGCTCTGAGAAACCTGGTGTATGGGAAGGCC
AACGATGATAACAAAATTGCCCTGAAAAACTGTGGTGGCATCCCAGCACTGGTGAGGTTACTCCGCAA
GACGACTGACCTGGAGATCCGGGAGCTGGTCACAGGAGTCCTTTGGAACCTCTCCTCATGCGATGCAC
TCAAAATGCCAATCATCCAGGATGCCCTAGCAGTACTGACCAACGCGGTGATTATCCCCCACTCAGGC
TGGGAAAATTCGCCTCTTCAGGATGATCGGAAAATACAGCTGCATTCATCACAGGTGCTGCGTAACGC
CACCGGGTGCCTAAGGAATGTTAGTTCGGCCGGAGAGGAGGCCCGCAGAAGGATGAGAGAGTGTGATG
GGCTTACGGATGCCTTGCTGTACGTGATCCAGTCTGCGCTGGGGAGCAGTGAGATCGATAGCAAGACC
GTTGAAAACTGTGTGTGCATTTTAAGGAACCTCTCGTACCGGCTGGCGGCAGAAACGTCTCAGGGACA
GCACATGGGCACGGACGAGCTGGACGGGCTACTCTGTGGCGAGGCCAATGGCAAGGATGCTGAGAGCT
CTGGGTGCTGGGGCAAGAAGAAGAAGAAAAAGAAATCCCAAGATCAGTGGGATGGAGTAGGACCTCTT
CCAGACTGTGCTGAACCACCAAAAGGGATCCAGATGCTGTGGCACCCATCAATAGTCAAACCCTACCT
CACACTGCTCTCTGAGTGCTCAAATCCAGACACGCTGGAAGGGGCGGCAGGCGCCCTGCAGAACTTGG
CTGCAGGGAGCTGGAAGGGCTGGGCTGAGGATGTGGCAGGCATGGCATATGCCCTACGTTCACTGCCA
GAGGGGGCTCCCTGCCTGCCACAGTGGTCAGTATATATCCGAGCCGCTGTCCGAAAAGAGAAAGGCCT
GCCCATCCTCGTGGAGCTGCTCCGAATAGACAATGACCGTGTGGTGTGCGCGGTGGCCACTGCGCTGC
GGAACATGGCCTTGGACGTCAGAAATAAGGAGCTCATCGGCAAATACGCCATGCGAGACCTAGTCCAC
AGGCTTCCAGGAGGGAACAACAGCAACAACACTGCAAGCAAGGCCATGTCGGATGACACAGTGACAGC
TGTCTGCTGCACACTGCACGAAGTGATTACCAAGAACATGGAGAACGCCAAGGCCTTACGGGATGCCG
GTGGCATCGAGAAGTTGGTCGGCATCTCCAAAAGCAAAGGAGATAAACACTCTCCAAAAGTGGTCAAG
GCTGCATCTCAGGTCCTCAACAGCATGTGGCAGTACCGAGATCTGAGGAGTCTCTACAAAAAGGATGG
ATGGTCACAATACCACTTTGTAGCCTCGTCTTCAACCATCGAGAGGGACCGGCAAAGGCCCTACTCCT
CCTCCCGCACGCCCTCCATCTCCCCTGTGCGCGTGTCTCCCAACAACCGCTCAGCAAGTGCCCCAGCT
TCACCTCGGGAAATGATCAGCCTCAAAGAAAGGAAAACAGACTACGAGTGCACCGGCAGCAACGCCAC
CTACCACGGAGCTAAAGGCGAACACACTTCCAGGAAAGATGCCATGACAGCTCAAAACACTGGAATTT
CAACTTTGTATAGGAATTCTTATGGTGCGCCCGCTGAAGACATCAAACACAACCAGGTTTCAGCACAG
CCAGTCCCACAGGAGCCCAGCAGAAAAGATTACGAGACCTACCAGCCATTTCAGAATTCCACAAGAAA
TTACGATGAGTCCTTCTTCGAGGACCAGGTCCACCATCGCCCTCCCGCCAGCGAGTACACCATGCACC
TGGGTCTCAAGTCCACCGGCAACTACGTTGACTTCTACTCAGCTGCCCGTCCCTACAGTGAACTGAAC
TATGAAACGAGCCACTACCCGGCCTCCCCCGACTCCTGGGTGTGAGGAGCAGGGCACAGGCGCTCCGG
GAACAGTGCATGTGCATGCATACCACAAGACATTTCTTTCTGTTTTGTTTTTTTCTCCTGCAAATTTA
GTTTGTTAAAGCCTGTTCCATAGGAAGGCTGTGATAACCAGTAAGGAAATATTAAGAGCTATTTTAGA
AAGCTAAATGAATCGCAAGTTAACTTGGAAATCAGTAGAAAGCTAAAGTGATCCTAAATATGACAGTG
GGCAGCACCTTTCTAGCGTGAGCTGTAGAGTAACGAGAAGTGCTTTATACTGAACGTGGTTGATGGGA
GGAGAGACGAGGCATTCGGGCCGGTGGGGCGTAAGGGTTATCGTTAAGCACAAGACACAGAATAGTTT
ACACACTGTGTGGGGGACGGCTTCTCACGCTTTGTTTACTCTCTTCATCCGTTGTGACTCTAGGCTTC
AGGTTGCATTGGGGTTCCTCTGTACAGCAAGATGTTTCTTGCCTTTTGTTAATGCATTGTTGTAAAGT
ATTTGATGTACATTACAGATTAAAGAAGAAAAGCGCGTTGTGTATATTACACCAATGCCGCCGTGTTT
CCTCATCTATGGTTCTAAATATTGCTTCAATTTCAAACTTTTGAAAGATGTATGGATTTCCAGTTTTT
CTTTACTTTCTCCCAGTATGTTTTAACAAAAAAAAAAAAAAGCAGGAAAAAAGGAATATTTAGCAGTA
TTGTTCGTTCTGATATGTGAATTTGTTTGTGACAACTAAACAAGGCATTCAGCAGTTTCTGACAATTA
ACATACATCATTCCACACTCCTTGTCAACAAAGTGCTTTTTCACTGCCTAAAATTTTAGATGTAGATA
TTTGAAATAGATTTTTTCATTTATACCAGTTTTCTTTATGATGATACAGTGTTAAAAGAAAATAAATT
ACAATTGATCTGTCATCCATATTTGCTAAGAATGTCTATTTCCAAGAACCTATATCTTACAAAATACA
CATTCTTGCTTGTGTGTCTGTGTGTGTGGGTGTATGTATGTCCACGTACTAGAAAAATTACCTGTGTG
AGATTTTATTTTGACAGAAGCTATTTCATTTGCTGTTCATCTTGTACAAACTTTGTTTTTGGTTTTTT
AGTATAAATGTACATCAGTTTGTTCCTTTTGTACTGTATCTATTCTTCTGACCATCTAGTGACTCAGG
ATATTAGGCCAGTTGAAGTGGAGTTATAGGATTTTCACACATTCACGCATACTTGGATATCAACCCTC
TCTACAGGTCCGTTTCCTTACACATTTTATCCCCTCAGAACACGATAAACCATGGCCAATTCAGTTTC
ACTTTGGGGCCAATTACTTGAAGTATCCACAGAAAATGTAAACTGCTCGCCTTATTCCCCCAAACTTT
TTGTTCATGTGGCAGGGAGCAGTACACAACTATGGAATAGATTATTAAAACTGAACTTGAAGCTAACG
TTAGAATAGTGGTTAACAGAGCTAAGAAATAAGGCTAACCATCGAAAAGAACATGGGCCGCGGGTAAA
CTAGCTTTGCTCTTTAGATGCAAGTGGCTGAGGCCAATAGGTGCCTAGTAAATGTTAACTGTTCTTGG
AAAAAATAAAAACATCAGTAACAAAAGAAA
Variant 6 Peptide from NM_001364128.2 Homo sapiens catenin delta 2
(CTNND2), transcript variant 6.
SEQ ID NO: 29.
MSSMSSAEEQFQWQSQDGQKDIEDELTTGLELVDSCIRSLQESGILDPQDYSTGETGSRASYSSQHGH
LGPELRALQSPEHHIDPIYEDRVYQKPPMRSLSQSQGDPLPPAHTGTYRTSTAPSSPGVDSVPLQRTG
SQHGPQNAAAATFQRASYAAGPASNYADPYRQLQYCPSVESPYSKSGPALPPEGTLARSPSIDSIQKD
PREFGWRDPELPEVIQMLQHQFPSVQSNAAAYLQHLCFGDNKIKAEIRRQGGIQLLVDLLDHRMTEVH
RSACGALRNLVYGKANDDNKIALKNCGGIPALVRLLRKTTDLEIRELVTGVLWNLSSCDALKMPIIQD
ALAVLTNAVIIPHSGWENSPLQDDRKIQLHSSQVLRNATGCLRNVSSAGEEARRRMRECDGLTDALLY
VIQSALGSSEIDSKTVENCVCILRNLSYRLAAETSQGQHMGTDELDGLLCGEANGKDAESSGCWGKKK
KKKKSQDQWDGVGPLPDCAEPPKGIQMLWHPSIVKPYLTLLSECSNPDTLEGAAGALQNLAAGSWKGW
AEDVAGMAYALRSLPEGAPCLPQWSVYIRAAVRKEKGLPILVELLRIDNDRVVCAVATALRNMALDVR
NKELIGKYAMRDLVHRLPGGNNSNNTASKAMSDDTVTAVCCTLHEVITKNMENAKALRDAGGIEKLVG
ISKSKGDKHSPKVVKAASQVLNSMWQYRDLRSLYKKDGWSQYHFVASSSTIERDRQRPYSSSRTPSIS
PVRVSPNNRSASAPASPREMISLKERKTDYECTGSNATYHGAKGEHTSRKDAMTAQNTGISTLYRNSY
GAPAEDIKHNQVSAQPVPQEPSRKDYETYQPFQNSTRNYDESFFEDQVHHRPPASEYTMHLGLKSTGN
YVDFYSAARPYSELNYETSHYPASPDSWV
Homo sapiens delta-catenin (Acc. No.: U96136.1); Transcript
length: 5305; CDS length: 3678; CDS start: 1; CDS end: 3678; peptide
length: 1225.
SEQ ID NO: 30.
ATGTTTGCGAGGAAGCCGCCGGGCGCCGCGCCTTTGGGAGCTATGCCTGTTCCAGACCAGCCTTCATC
AGCCTCAGAGAAGACGAGTTCCCTGAGCCCCGGCTTAAACACCTCCAACGGGGATGGCTCTGAAACAG
AAACCACCTCTGCCATCCTCGCCTCAGTCAAAGAACAGGAATTACAGTTTGAAAGGCTGACCCGAGAG
CTGGAGGCTGAACGGCAGATCGTAGCCAGCCAGCTGGAGCGATGCAAGCTCGGATCCGAGACTGGCAG
CATGAGCAGCATGAGTTCAGCAGAAGAGCAGTTTCAGTGGCAGTCACAAGATGGTCAAAAAGATATCG
AAGATGAGCTTACAACAGGTCTCGAGCTGGTGGACTCCTGTATTAGGTCACTACAGGAATCAGGAATA
CTTGACCCACAGGATTATTCTACAGGTGAAAGGCCCAGCCTGCTCTCCCAGAGTGCACTTCAGCTCAA
TTCCAAACCTGAAGGGTCTTTCCAGTATCCGGCCAGCTACCATAGCAACCAGACCCTGGCCCTGGGGG
AAACCACCCCTTCACAGCTCCCGGCCCGAGGCACACAAGCCCGAGCTACGGGCCAGAGCTTCAGCCAG
GGCACGACCAGCCGCGCCGGCCACCTGGCGGGGCCCGAGCCCGCGCCGCCGCCGCCGCCGCCGCCGCG
GGAGCCGTTCGCGCCCAGCCTGGGCAGCGCCTTCCACCTGCCCGACGCGCCGCCCGCCGCCGCCGCCG
CCGCGCTCTACTACTCCAGCTCCACGCTGCCCGCGCCGCCGCGCGGGGGCTCCCCGCTGGCCGCGCCC
CAGGGCGGTTCGCCCACCAAGCTGCAGCGCGGCGGCTCGGCCCCCGAGGGCGCCACCTACGCCGCGCC
GCGCGGCTCCTCGCCCAAGCAGTCGCCCAGCCGCCTGGCCAAGTCCTACAGCACCAGCTCGCCCATCA
ACATCGTCGTGTCCTCGGCCGGCCTGTCCCCGATCCGCGTGACCTCGCCCCCCACCGTGCAGTCCACC
ATCTCCTCCTCGCCCATCCACCAGCTGAGCTCCACCATCGGCACGTACGCCACCCTGTCGCCCACCAA
GCGCCTGGTCCACGCGTCCGAGCAGTACAGCAAGCACTCGCAGGAGCTGTATGCCACGGCCACCCTCC
AGAGGCCGGGCAGCCTGGCAGCTGGTTCCCGAGCCTCATACAGCAGCCAGCATGGGCACCTGGGCCCA
GAGTTGCGGGCCCTGCAGTCCCCAGAACACCACATAGATCCCATCTATGAAGTCCGCGTCTATCAGAA
GCCCCCTATGAGGAGTCTCAGCCAGAGCCAGGGGGTCCCTCTGCCGCCAGCACACACCGGCACCTACC
GCACGAGCACAGCCCCATCTTCCCCTGGTGTCGACTCCGTCCCCTTGCAGCGCACAGGCAGCCAGCAC
GGCCCACAGAATGCCGCCGCGGCCACCTTCCAGAGGGCCAGCTATGCCGCCGGCCCAGCCTCCAATTA
CGCGGACCCCTACCGACAGCTGCAGTATTGTCCCTCTGTTGAGTCTCCATACAGCAAATCCGGCCCTG
CTCTCCCGCCTGAAGGCACCTTGGCCAGGTCCCCGTCCATTGATAGCATTCAGAAAGATCCCAGAGAA
TTTGGATGGAGAGACCCGGAACTGCCGGAAGTGATTCAGATGTTGCAGCACCAGTTTCCCTCGGTCCA
GTCTAACGCGGCAGCCTACTTGCAACACCTCTGTTTTGGAGACAACAAAATTAAAGCCGAGATAAGGA
GACAAGGAGGCATCCAGCTCCTGGTGGACCTGTTGGATCATCGGATGACCGAAGTCCACCGTAGTGCC
TGTGGAGCTCTGAGAAACCTGGTGTATGGGAAGGCCAACGATGATAACAAAATTGCCCTGAAAAACTG
TGGTGGCATCCCAGCACTGGTGAGGTTACTCCGCAAGACGACTGACCTGGAGATCCGGGAGCTGGTCA
CAGGAGTCCTTTGGAACCTCTCCTCATGCGATGCACTCAAAATGCCAATCATCCAGGATGCCCTAGCA
GTACTGACCAACGCGGTGATTATCCCCCACTCAGGCTGGGAAAATTCGCCTCTTCAGGATGATCGGAA
AATACAGCTGCATTCATCACAGGTGCTGCGTAACGCCACCGGGTGCTTAAGGAATGTTAGTTCGCCCG
GAGAGGAGGCCCGCAGAAGGATGAGAGAGTGTGATGGGCTTACGGATGCCTTGCTGTACGTGATCCAG
TCTGCGCTGGGGAGCAGTGAGATCGATAGCAAGACCGTTGAAAACTGTGTGTGCATTTTAAGGAACCT
CTCGTACCGGCTGGCGGCAGAAACGTCTCAGGGACAGCACATGGGCACGGACGAGCTGGACGGGCTAC
TCTGTGGCGAGGCCAATGGCAAGGATGCTGAGAGCTCTGGGTGCTGGGGCAAGAAGAAGAAGAAAAAG
AAATCCCAAGATCAGTGGGATGGAGTAGGACCTCTTCCAGACTGTGCTGAACCACCAAAAGGGATCCA
GATGCTGTGGCACCCATCAATAGTCAAACCCTACCTCACACTGCTCTCTGAGTGCTCAAATCCAGACA
CGCTGGAAGGGGCGGCAGGCGCCCTGCAGAACTTGGCTGCAGGGAGCTGGAAGTGGTCAGTATATATC
CGAGCCGCTGTCCGAAAAGAGAAAGGCCGGCCCATCCTCGTGGAGCTGCTCCGAATAGACAATGACCG
TGTGGCGTGCGCGGTGGCCACTGCGCTGCGGAACATGGCCTTGGACGTCAGAAATAAGGAGCTCATCG
GCAAATACGCCATGCGAGACCTAGTCCACAGGCTTCCAGGAGGGAACAACAGCAACAACACTGCAAGC
AAGGCCATGTCGGATGACACAGTGACAGCTGTCTGCTGCACACTGCACGAAGTGATTACCAAGAACAT
GGAGAACGCCAAGGCCTTACGGGATGCCGGTGGCATCGAGAAGTTGGTCGGCATCTCCAAAAGCAAAG
GAGATAAACACTCTCCAAAAGTGGTCAAGGCTGCATCTCAGGTCCTCAACAGCATGTGGCAGTACCGA
GATCTGAGGAGTCTCTACAAAAAGGATGGATGGTCACAATACCACTTTGTAGCCTCGTCTTCAACCAT
CGAGAGGGACCGGCAAAGGCCCTACTCCTCCTCCCGCACGCCCTCCATCTCCCCTGTGCGCGTGTCTC
CCAACAACCGCTCAGCAAGTGCCCCAGCTTCACCTCGGGAAATGATCAGCCTCAAAGAAAGGAAAACA
GACTACGAGTGCACCGGCAGCAACGCCACCTACCACGGAGCTAAAGGCGAACACACTTCCAGGAAAGA
TGCCATGACAGCTCAAAACACTGGAATTTCAACTTTGTATAGGAATTCTTATGGTGCGCCCGCTGAAG
ACATCAAACACAACCAGGTTTCAGCACAGCCAGTCCCACAGGAGCCCAGCAGAAAAGATTACGAGACC
TACCAGCCATTTCAGAATTCCACAAGAAATTACGATGAGTCCTTCTTCGAGGACCAGGTCCACCATCG
CCCTCCCGCCAGCGAGTACACCATGCACCTGGGTCTCAAGTCCACCGGCAACTACGTTGACTTCTACT
CAGCTGCCCGTCCCTACAGTGAACTGAACTATGAAACGAGCCACTACCCGGCCTCCCCCGACTCCTGG
GTGTGAGGAGCAGGGCACAGGCGCTCCGGGAACAGTGCATGTGCATGCATACCACAAGACATTTCTTT
CTGTTTTGTTTTTTTCTCCTGCAAATTTAGTTTGTTAAAGCCTGTTCCATAGGAAGGCTGTGATAACC
AGTAAGGAAATATTAAGAGCTATTTTAGAAAGCTAAATGAATCGCAAGTTTAACTTGGAAATCAGTAG
AAAGCTAAAGTGATCCTAAATATGACAGTGGGCAGCACCTTTCTAGCGTGAGCTGTAAAGTAACGAGA
AGTGCTTTATACTGAACGTGGTTGATGGGAGGAGAGACGAGGCATTCGGGCCGGTGGGGCGTAAGGGT
TATCGTTAAGCACAAGACACAGAATAGTTTACACACTGTGTGGGGGACGGCTTCTCACGCTTTGTTTA
CTCTCTTCATCCGTTGTGACTCTAGGCTTCAGGTTGCATTGGGGTTCCTCTGTACAGCAAGATGTTTC
TTGCCTTTTGTTAATGCATTGTTGTAAAGTATTTGATGTACATTACAGATTAAAGAAGAAAAGCGCGT
TGTGTATATTACACCAATGCCGCCGTGTTTCCTCATCTATGGTTCTAAATATTGCTTCAATTTCAAAC
TTTTGAAAGATGTATGGATTTCCAGTTTTTCTTTACTTTCTCCCAGTATGTTTTAACAAAAAAAAAAA
AAAGCAGGAAAAAAGGAATATTTAGCAGTATTGTTCGTTCTGATATGTGAATTTGTTTGTGACAACTA
AACAAGGCATTCAGCAGTTTCTGACAATTAACATACATCATTCCACACTCCTTGTCAACAAAGTGCTT
TTTCACTGCCTAAAATTTTAGATGTAGATATTTGAAATAGATTTTTTCATTTATACCAGTTTTCTTTA
TGATGATACAGTGTTAAAAGAAAATAAATTACAATTGATCTGTCATCCATATTTGCTAAGAATGTCTA
TTTCCAAGAACCTATATCTTACAAAATACACATTCTTGCTTGTGTGTCTGTGTGTGTGGGTGTATGTA
TGTCCACGTACTAGAAAAATTACCTGTGTGAGATTTTATTTTGACAGAAGCTATTTCATTTGCTGTTC
ATCTTGTACAAACTTTGTTTTTGGTTTTTTAGTATAAATGTACATCAGTTTGTTCCTTTTGTACTGTA
TCTATTCTTCTGACCATCTAGTGACTCAGGATATTAGGCCCAGTTGAAGTGGAGTTATGGGGATTTTC
CACACATTCACGCATACTTGGATATCAACCCTCTCTACAGGTCCGTTTCCTTACACATTTTATCCCCC
TCAGAACACGATAAACCATGGCCAATTCAGTTTCACTTTGGGGCCCAATTACTTGAAGTATCCCACAG
AAAATGTAAACTGCTCGCCTTATTCCCCCAAACTTTTTGTTCATGTGGCAGGGAGCAGTACACAACTA
TGGAATAGATTATTAAAACTGAACTTGAAGCTAACGTTAGAATAGTGGTTAACAGAGCTAAGAAATAA
GGCTAACCATCGAAAAGAACATGGGCCGCGGGTAAACTAGCTTTGCTCTTTAGATGCAAGTGGCTGAG
GCCAATAGGTGCCTAGTAAATGTTAACTGTTCTTGGAAAAAATAAAAACATCAGTAACAAAAAAAAAA
A
Peptide from Homo sapiens delta-catenin (Acc. No.: U96136.1).
SEQ ID NO: 31.
MFARKPPGAAPLGAMPVPDQPSSASEKTSSLSPGLNTSNGDGSETETTSAILASVKEQELQFERLTRE
LEAERQIVASQLERCKLGSETGSMSSMSSAEEQFQWQSQDGQKDIEDELTTGLELVDSCIRSLQESGI
LDPQDYSTGERPSLLSQSALQLNSKPEGSFQYPASYHSNQTLALGETTPSQLPARGTQARATGQSFSQ
GTTSRAGHLAGPEPAPPPPPPPREPFAPSLGSAFHLPDAPPAAAAAALYYSSSTLPAPPRGGSPLAAP
QGGSPTKLQRGGSAPEGATYAAPRGSSPKQSPSRLAKSYSTSSPINIVVSSAGLSPIRVTSPPTVQST
ISSSPIHQLSSTIGTYATLSPTKRLVHASEQYSKHSQELYATATLQRPGSLAAGSRASYSSQHGHLGP
ELRALQSPEHHIDPIYEDRVYQKPPMRSLSQSQGDPLPPAHTGTYRTSTAPSSPGVDSVPLQRTGSQH
GPQNAAAATFQRASYAAGPASNYADPYRQLQYCPSVESPYSKSGPALPPEGTLARSPSIDSIQKDPRE
FGWRDPELPEVIQMLQHQFPSVQSNAAAYLQHLCFGDNKIKAEIRRQGGIQLLVDLLDHRMTEVHRSA
CGALRNLVYGKANDDNKIALKNCGGIPALVRLLRKTTDLEIRELVTGVLWNLSSCDALKMPIIQDALA
VLTNAVIIPHSGWENSPLQDDRKIQLHSSQVLRNATGCLRNVSSAGEEARRRMRECDGLTDALLYVIQ
SALGSSEIDSKTVENCVCILRNLSYRLAAETSQGQHMGTDELDGLLCGEANGKDAESSGCWGKKKKKK
KSQDQWDGVGPLPDCAEPPKGIQMLWHPSIVKPYLTLLSECSNPDTLEGAAGALQNLAAGSWKWSVYI
RAAVRKEKGLPILVELLRIDNDRVVCAVATALRNMALDVRNKELIGKYAMRDLVHRLPGGNNSNNTAS
KAMSDDTVTAVCCTLHEVITKNMENAKALRDAGGIEKLVGISKSKGDKHSPKVVKAASQVLNSMWQYR
DLRSLYKKDGWSQYHFVASSSTIERDRQRPYSSSRTPSISPVRVSPNNRSASAPASPREMISLKERKT
DYECTGSNATYHGAKGEHTSRKDAMTAQNTGISTLYRNSYGAPAEDIKHNQVSAQPVPQEPSRKDYET
YQPFQNSTRNYDESFFEDQVHHRPPASEYTMHLGLKSTGNYVDFYSAARPYSELNYETSHYPASPDSW
V
S276A NUCLEOTIDE SEQUENCE; ORF; TCG to GCA, Serine to Alanine).
SEQ ID NO: 32
ATGTTTGCGAGGAAGCCGCCGGGCGCCGCGCCTTTGGGAGCTATGCCTGTTCCAGACCAGCCTTCATC
AGCCTCAGAGAAGACGAGTTCCCTGAGCCCCGGCTTAAACACCTCCAACGGGGATGGCTCTGAAACAG
AAACCACCTCTGCCATCCTCGCCTCAGTCAAAGAACAGGAATTACAGTTTGAAAGGCTGACCCGAGAG
CTGGAGGCTGAACGGCAGATCGTAGCCAGCCAGCTGGAGCGATGCAAGCTCGGATCCGAGACTGGCAG
CATGAGCAGCATGAGTTCAGCAGAAGAGCAGTTTCAGTGGCAGTCACAAGATGGTCAAAAAGATATCG
AAGATGAGCTTACAACAGGTCTCGAGCTGGTGGACTCCTGTATTAGGTCACTACAGGAATCAGGAATA
CTTGACCCACAGGATTATTCTACAGGTGAAAGGCCCAGCCTGCTCTCCCAGAGTGCACTTCAGCTCAA
TTCCAAACCTGAAGGGTCTTTCCAGTATCCGGCCAGCTACCATAGCAACCAGACCCTGGCCCTGGGGG
AAACCACCCCTTCACAGCTCCCGGCCCGAGGCACACAAGCCCGAGCTACGGGCCAGAGCTTCAGCCAG
GGCACGACCAGCCGCGCCGGCCACCTGGCGGGGCCCGAGCCCGCGCCGCCGCCGCCGCCGCCGCCGCG
GGAGCCGTTCGCGCCCAGCCTGGGCAGCGCCTTCCACCTGCCCGACGCGCCGCCCGCCGCCGCCGCCG
CCGCGCTCTACTACTCCAGCTCCACGCTGCCCGCGCCGCCGCGCGGGGGCTCCCCGCTGGCCGCGCCC
CAGGGCGGTGCACCCACCAAGCTGCAGCGCGGCGGCTCGGCCCCCGAGGGCGCCACCTACGCCGCGCC
GCGCGGCTCCTCGCCCAAGCAGTCGCCCAGCCGCCTGGCCAAGTCCTACAGCACCAGCTCGCCCATCA
ACATCGTCGTGTCCTCGGCCGGCCTGTCCCCGATCCGCGTGACCTCGCCCCCCACCGTGCAGTCCACC
ATCTCCTCCTCGCCCATCCACCAGCTGAGCTCCACCATCGGCACGTACGCCACCCTGTCGCCCACCAA
GCGCCTGGTCCACGCGTCCGAGCAGTACAGCAAGCACTCGCAGGAGCTGTATGCCACGGCCACCCTCC
AGAGGCCGGGCAGCCTGGCAGCTGGTTCCCGAGCCTCATACAGCAGCCAGCATGGGCACCTGGGCCCA
GAGTTGCGGGCCCTGCAGTCCCCAGAACACCACATAGATCCCATCTATGAAGACCGCGTCTATCAGAA
GCCCCCTATGAGGAGTCTCAGCCAGAGCCAGGGGGACCCTCTGCCGCCAGCACACACCGGCACCTACC
GCACGAGCACAGCCCCATCTTCCCCTGGTGTCGACTCCGTCCCCTTGCAGCGCACAGGCAGCCAGCAC
GGCCCACAGAATGCCGCCGCGGCCACCTTCCAGAGGGCCAGCTATGCCGCCGGCCCAGCCTCCAATTA
CGCGGACCCCTACCGACAGCTGCAGTATTGTCCCTCTGTTGAGTCTCCATACAGCAAATCCGGCCCTG
CTCTCCCGCCTGAAGGCACCTTGGCCAGGTCCCCGTCCATTGATAGCATTCAGAAAGATCCCAGAGAA
TTTGGATGGAGAGACCCGGAACTGCCGGAAGTGATTCAGATGTTGCAGCACCAGTTTCCCTCGGTCCA
GTCTAACGCGGCAGCCTACTTGCAACACCTCTGTTTTGGAGACAACAAAATTAAAGCCGAGATAAGGA
GACAAGGAGGCATCCAGCTCCTGGTGGACCTGTTGGATCATCGGATGACCGAAGTCCACCGTAGTGCC
TGTGGAGCTCTGAGAAACCTGGTGTATGGGAAGGCCAACGATGATAACAAAATTGCCCTGAAAAACTG
TGGTGGCATCCCAGCACTGGTGAGGTTACTCCGCAAGACGACTGACCTGGAGATCCGGGAGCTGGTCA
CAGGAGTCCTTTGGAACCTCTCCTCATGCGATGCACTCAAAATGCCAATCATCCAGGATGCCCTAGCA
GTACTGACCAACGCGGTGATTATCCCCCACTCAGGCTGGGAAAATTCGCCTCTTCAGGATGATCGGAA
AATACAGCTGCATTCATCACAGGTGCTGCGTAACGCCACCGGGTGCCTAAGGAATGTTAGTTCGGCCG
GAGAGGAGGCCCGCAGAAGGATGAGAGAGTGTGATGGGCTTACGGATGCCTTGCTGTACGTGATCCAG
TCTGCGCTGGGGAGCAGTGAGATCGATAGCAAGACCGTTGAAAACTGTGTGTGCATTTTAAGGAACCT
CTCGTACCGGCTGGCGGCAGAAACGTCTCAGGGACAGCACATGGGCACGGACGAGCTGGACGGGCTAC
TCTGTGGCGAGGCCAATGGCAAGGATGCTGAGAGCTCTGGGTGCTGGGGCAAGAAGAAGAAGAAAAAG
AAATCCCAAGATCAGTGGGATGGAGTAGGACCTCTTCCAGACTGTGCTGAACCACCAAAAGGGATCCA
GATGCTGTGGCACCCATCAATAGTCAAACCCTACCTCACACTGCTCTCTGAGTGCTCAAATCCAGACA
CGCTGGAAGGGGCGGCAGGCGCCCTGCAGAACTTGGCTGCAGGGAGCTGGAAGTGGTCAGTATATATC
CGAGCCGCTGTCCGAAAAGAGAAAGGCCTGCCCATCCTCGTGGAGCTGCTCCGAATAGACAATGACCG
TGTGGTGTGCGCGGTGGCCACTGCGCTGCGGAACATGGCCTTGGACGTCAGAAATAAGGAGCTCATCG
GCAAATACGCCATGCGAGACCTAGTCCACAGGCTTCCAGGAGGGAACAACAGCAACAACACTGCAAGC
AAGGCCATGTCGGATGACACAGTGACAGCTGTCTGCTGCACACTGCACGAAGTGATTACCAAGAACAT
GGAGAACGCCAAGGCCTTACGGGATGCCGGTGGCATCGAGAAGTTGGTCGGCATCTCCAAAAGCAAAG
GAGATAAACACTCTCCAAAAGTGGTCAAGGCTGCATCTCAGGTCCTCAACAGCATGTGGCAGTACCGA
GATCTGAGGAGTCTCTACAAAAAGGATGGATGGTCACAATACCACTTTGTAGCCTCGTCTTCAACCAT
CGAGAGGGACCGGCAAAGGCCCTACTCCTCCTCCCGCACGCCCTCCATCTCCCCTGTGCGCGTGTCTC
CCAACAACCGCTCAGCAAGTGCCCCAGCTTCACCTCGGGAAATGATCAGCCTCAAAGAAAGGAAAACA
GACTACGAGTGCACCGGCAGCAACGCCACCTACCACGGAGCTAAAGGCGAACACACTTCCAGGAAAGA
TGCCATGACAGCTCAAAACACTGGAATTTCAACTTTGTATAGGAATTCTTATGGTGCGCCCGCTGAAG
ACATCAAACACAACCAGGTTTCAGCACAGCCAGTCCCACAGGAGCCCAGCAGAAAAGATTACGAGACC
TACCAGCCATTTCAGAATTCCACAAGAAATTACGATGAGTCCTTCTTCGAGGACCAGGTCCACCATCG
CCCTCCCGCCAGCGAGTACACCATGCACCTGGGTCTCAAGTCCACCGGCAACTACGTTGACTTCTACT
CAGCTGCCCGTCCCTACAGTGAACTGAACTATGAAACGAGCCACTACCCGGCCTCCCCCGACTCCTGG
GTGTGA
delta-catenin S276A Peptide Sequence.
SEQ ID NO: 33
MFARKPPGAAPLGAMPVPDQPSSASEKTSSLSPGLNTSNGDGSETETTSAILASVKEQELQFERLTRE
LEAERQIVASQLERCKLGSETGSMSSMSSAEEQFQWQSQDGQKDIEDELTTGLELVDSCIRSLQESGI
LDPQDYSTGERPSLLSQSALQLNSKPEGSFQYPASYHSNQTLALGETTPSQLPARGTQARATGQSFSQ
GTTSRAGHLAGPEPAPPPPPPPREPFAPSLGSAFHLPDAPPAAAAAALYYSSSTLPAPPRGGSPLAAP
QGGAPTKLQRGGSAPEGATYAAPRGSSPKQSPSRLAKSYSTSSPINIVVSSAGLSPIRVTSPPTVQST
ISSSPIHQLSSTIGTYATLSPTKRLVHASEQYSKHSQELYATATLQRPGSLAAGSRASYSSQHGHLGP
ELRALQSPEHHIDPIYEDRVYQKPPMRSLSQSQGDPLPPAHTGTYRTSTAPSSPGVDSVPLQRTGSQH
GPQNAAAATFQRASYAAGPASNYADPYRQLQYCPSVESPYSKSGPALPPEGTLARSPSIDSIQKDPRE
FGWRDPELPEVIQMLQHQFPSVQSNAAAYLQHLCFGDNKIKAEIRRQGGIQLLVDLLDHRMTEVHRSA
CGALRNLVYGKANDDNKIALKNCGGIPALVRLLRKTTDLEIRELVTGVLWNLSSCDALKMPIIQDALA
VLTNAVIIPHSGWENSPLQDDRKIQLHSSQVLRNATGCLRNVSSAGEEARRRMRECDGLTDALLYVIQ
SALGSSEIDSKTVENCVCILRNLSYRLAAETSQGQHMGTDELDGLLCGEANGKDAESSGCWGKKKKKK
KSQDQWDGVGPLPDCAEPPKGIQMLWHPSIVKPYLTLLSECSNPDTLEGAAGALQNLAAGSWKWSVYI
RAAVRKEKGLPILVELLRIDNDRVVCAVATALRNMALDVRNKELIGKYAMRDLVHRLPGGNNSNNTAS
KAMSDDTVTAVCCTLHEVITKNMENAKALRDAGGIEKLVGISKSKGDKHSPKVVKAASQVLNSMWQYR
DLRSLYKKDGWSQYHFVASSSTIERDRQRPYSSSRTPSISPVRVSPNNRSASAPASPREMISLKERKT
DYECTGSNATYHGAKGEHTSRKDAMTAQNTGISTLYRNSYGAPAEDIKHNQVSAQPVPQEPSRKDYET
YQPFQNSTRNYDESFFEDQVHHRPPASEYTMHLGLKSTGNYVDFYSAARPYSELNYETSHYPASPDSW
V

1. A recombinant transgene comprising a polynucleotide that encodes human-delta-catenin protein, or a variant thereof.

2. The transgene of claim 1, wherein the polynucleotide is selected from the group consisting of SEQ ID NO:20, SEQ ID NO:22, SEQ ID NO:24, SEQ ID NO:26, SEQ ID NO:28, or SEQ ID NO:30.

3. The transgene according to claim 1, wherein the polynucleotide encodes the human-delta-catenin (S276A) protein, or a variant thereof.

4. The transgene of claim 3, wherein the polynucleotide comprises SEQ ID NO:32.

5. An expression cassette comprising a polynucleotide sequence encoding the human-delta-catenin protein, or a variant thereof.

6. The expression cassette of claim 5, wherein the polynucleotide sequence is selected from the group consisting of SEQ ID NO:20, SEQ ID NO:22, SEQ ID NO:24, SEQ ID NO:26, SEQ ID NO:28, or SEQ ID NO:30.

7. The expression cassette of claim 6, wherein the polynucleotide sequence has at least 95% sequence identity to any one of SEQ ID NO:20, SEQ ID NO:22, SEQ ID NO:24, SEQ ID NO:26, SEQ ID NO:28, or SEQ ID NO:30

8. The expression cassette according to claim 5, wherein the polynucleotide sequence encodes the human-delta-catenin (S276A) protein, or a variant thereof.

9. The expression cassette of claim 8, wherein the polynucleotide sequence comprises SEQ ID NO:32.

10. The expression cassette according to any one of claims 5-9, wherein the expression cassette further comprises a polynucleotide sequence that is codon-optimized to reduce CpG methylation sites and mammalian expression encoding the human-delta-catenin transgene.

11. The expression cassette as in any one of claim 5-10, wherein the cassette comprises the human-delta-catenin transgene sequence operably linked to a promoter and a polyadenylation sequence.

12. The expression cassette according to claim 11, wherein the promoter is selected from the group consisting of synapsin 1, calcium/calmodulin-dependent protein kinase II, tubulin alpha 1, neuron-specific enolase, human KCC2 promoter, or platelet-derived growth factor beta chain promoters.

13. The expression cassette according to claim 12, wherein the promoter comprises a synapsin I promoter.

14. The expression cassette according to claim 13, wherein the promoter comprise a human KCC2 promoter.

15. The expression cassette according to claim 11, wherein the expression cassette comprise a constitutively active promoter.

16. The expression cassette according to claim 11, wherein the expression cassette comprises a neuron-specific promoter.

17. The expression cassette according to either of claims 15 or 16, wherein in which the promoter is selected from the group consisting of human β-actin, human elongation factor-1α, chicken β-actin combined with cytomegalovirus early enhancer, cytomegalovirus (CMV), simian virus 40, and herpes simplex virus thymidine kinase.

18. The expression cassette as in any one of claims 5-17, wherein the nucleic acid expression cassette further comprises a transcriptional termination signal selected from the group consisting of bovine growth hormone polyadenylation signal (BGHpA), Simian virus 40 polyadenylation signal (SV40pA), and a synthetic polyadenylation signal.

19. A recombinant viral vector comprising a polynucleotide sequence encoding the gene for human-delta-catenin, or a fragment, isoform, or homologue thereof.

20. A recombinant viral vector comprising an expression cassette as in any one of claims 5 to 18.

21. The recombinant viral vector of either of claims 19 or 20, the vector further comprising one or more of the following elements: (a) an inverted terminal repeat sequence (ITR); (b) a promoter; (c) an intron; (d) transcription terminator; and (e) a flanking inverted terminal repeat sequence (ITR).

22. The recombinant viral vector according to claim 21, wherein the promoter is selected from the group consisting of synapsin 1, calcium/calmodulin-dependent protein kinase II, tubulin alpha 1, neuron-specific enolase, human KCC2 promoter, or platelet-derived growth factor beta chain promoters.

23. The recombinant viral vector according to claim 22, wherein the promoter comprises a synapsin I promoter.

24. The recombinant viral vector according to claim 22, wherein the promoter comprise a human KCC2 promoter.

25. The recombinant viral vector according to claim 21, wherein the promoter comprises a constitutively active promoter.

26. The recombinant viral vector according to claim 21, wherein the expression cassette comprises a neuron-specific promoter.

27. The recombinant viral vector according to claim 25, wherein the promoter is selected from the group consisting of human β-actin, human elongation factor-1α, chicken β-actin combined with cytomegalovirus early enhancer, cytomegalovirus (CMV), simian virus 40, and herpes simplex virus thymidine kinase.

28. The recombinant viral vector as in any one of claims 18-27, wherein the recombinant viral vector further comprises a transcriptional termination signal selected from the group consisting of bovine growth hormone polyadenylation signal (BGHpA), Simian virus 40 polyadenylation signal (SV40pA), and a synthetic polyadenylation signal.

29. The recombinant viral vector as in any one of claims 18-28, wherein the recombinant viral vector is selected from the group consisting of adenoviruses, Adeno-associated viruses (AAV), Herpes simplex viruses (e.g., Herpes Simplex Virus Type 1), Retroviruses, lentiviruses, alphaviruses, flaviviruses, rhabdoviruses, measles virus, Newcastle disease virus, poxviruses, and picornaviruses.

30. The recombinant viral vector according to claim 29, wherein the recombinant viral vector comprises Adeno-associated virus (AAV).

31. The recombinant AAV vector according to claim 30, wherein the recombinant AAV vector comprises a serotype selected from the group consisting of AAV1, AAV2, AAV4, AAV5, AAV6, AAV7, AAVrh74, AAV8, AAV9, AAV10, AAV11, AAV12, or AAV13.

32. The recombinant AAV vector according to claim 31, wherein the recombinant AAV vector is selected from the group consisting of AAV1, AAV8, and AAV9.

33. The recombinant AAV vector according to claim 32, wherein the recombinant AAV vector comprises AAV9.

34. The recombinant AAV vector according to claim 32, wherein the recombinant AAV vector comprises AAV1.

35. A composition comprising a transgene as in any one of claims 1-4.

36. A composition comprising an expression cassette as in any one of claims 5-18.

37. A composition comprising a recombinant viral vector as in any one of claims 19-34.

38. A pharmaceutical composition comprising a composition as in any one of claims 35-37 and a pharmaceutically acceptable carrier and/or excipient.

39. A method of treating pain in a subject in need thereof, the method comprising administering to the subject a therapeutically effective amount of a composition as in any one of claims 35-38 such that the pain is treated in the subject.

40. A method of treating itch in a subject in need thereof, the method comprising administering to the subject a therapeutically effective amount of a composition as in any one of claims 35-38 such that the itch is treated in the subject.

41. A method of increasing KCC2 mRNA levels in a subject in need thereof, comprising administering to the subject a therapeutically effective amount of a composition as in any one of claims 35-38 such that the KCC2 mRNA levels are increased in the subject.

42. The method of claim 41, wherein KCC2mRNA levels are increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline.

43. The method of claim 42, wherein KCC2mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

44. A method of reducing intracellular chloride ion levels ([Cl⁻]i) in a central nervous system cell in a subject in need thereof, comprising administering to the subject a therapeutically effective amount of a composition as in any one of claims 35-38 such that the [Cl⁻]i in a central nervous system cell is reduced in the subject.

45. The method of claim 44, wherein [Cl⁻]i levels are reduced at least about 10%, or at least about 20%, or at least about 30%, or at least about 40%, or at least about 50%, or at least about 60%, or at least about 70%, or at least about 80%, or at least about 90% compared to pre-treatment baseline.

46. A method of increasing chloride ion efflux in a central nervous system cell in a subject in need thereof, comprising administering to the subject a therapeutically effective amount of a composition as in any one of claims 35-38 such that the chloride ion efflux is increased in the subject.

47. The method of claim 46, wherein chloride ion efflux levels are increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline.

48. A method of increasing synaptophysin expression in a central nervous system cell in a subject in need thereof, the method comprising administering to the subject a therapeutically effective amount of a composition as in any one of claims 35-38 such that the synaptophysin expression is increased in the subject.

49. The method of claim 48, wherein synaptophysin expression is increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline.

50. The method of claim 49, wherein synaptophysin expression is increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

51. A method of increasing KCC2 expression in a subject in need thereof, the method comprising administering to the subject a therapeutically effective amount of a composition as in any one of claims 35-38 such that KCC2 expression is increased in the subject.

52. The method of claim 51, wherein KCC2 expression is increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline.

53. The method of claim 52, wherein KCC2 mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

54. The method as in any one of claims 39-53, the method further comprising administering to the subject a therapeutically effective amount of at least one additional compound.

55. The method according to claim 54, wherein the additional compound is selected from the group consisting of a GSK3β inhibitor, an anti-analgesic, a muscle relaxant, an anti-anxiety drug, an antidepressant, an anticonvulsant, and combinations thereof.

56. The method according to claim 54, wherein the additional compound is selected from the group consisting of a GSK3β inhibitor a corticosteroid, a counterirritant, an antihistamine, and a local anesthetic and combinations thereof.

57. The method as in either of claims 55 or 56, wherein the GSK3β inhibitor is selected from the group consisting of CHIR-99021 (CT99021) HCl, SB-216763, CHIR-98014, TWS119, Tideglusib, SB-415286, BIO (6-bromoindirubin-3′-oxime), kenpaullone, CHIR-99021 (CT99021), AZD2858, AZD1080, AR-A014418, TDZD-8, LY2090314, IM-12, BIO-acetoxime, Indirubin, 5-Bromoindole, 2-D08, Bilinin, 1-Azakenpaullone, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium and any combinations thereof.

58. The method of claim 57, wherein the GSK3β inhibitor is selected from the group consisting of kenpaullone (9-bromo-7,12-dihydro-indolo [3,2-d] [1]benzazepin-6(5H)-one), NSC180515 (2-Acetyl-2,3,4,5-tetrahydrooxonine-6,9-dione), NSC79456 (n-(2,4-Dimethylphenyl)-2-hydroxy-3-nitrobenzamide), or NSC33006 (N-[4-(1,3-benzothiazol-2-yl)phenyl]acetamide).

59. The method of claim 58, wherein the GSK3β inhibitor is kenpaullone (9-bromo-7,12-dihydro-indolo [3,2-d] [1]benzazepin-6(5H)-one).

60. A method of treating pain in a subject in need thereof, the method comprising administering to the subject a therapeutically effective amount of a GSK3β inhibitor, or a pharmaceutical composition comprising a GSK3β inhibitor and a pharmaceutically acceptable carrier and/or excipient, such that the pain is treated in the subject.

61. A method of treating itch in a subject in need thereof, the method comprising, consisting of, or consisting essentially of administering to the subject a therapeutically effective amount of a GSK3β inhibitor, or a pharmaceutical composition comprising a GSK3β inhibitor and a pharmaceutically acceptable carrier and/or excipient, such that the itch is treated in the subject.

62. A method of increasing KCC2 mRNA levels in a subject in need thereof, the method comprising administering to the subject a therapeutically effective amount of a GSK3p inhibitor, or a pharmaceutical composition comprising a GSK3β inhibitor and a pharmaceutically acceptable carrier and/or excipient, such that the KCC2 mRNA levels are increased in the subject.

63. The method of claim 62, wherein KCC2 mRNA levels are increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline.

64. The method of claim 63, wherein KCC2mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

65. A method of reducing intracellular chloride ion levels ([Cl⁻]i) in a central nervous system cell in a subject in need thereof, the method comprising administering to the subject a therapeutically effective amount of a GSK3β inhibitor, or a pharmaceutical composition comprising a GSK3β inhibitor and a pharmaceutically acceptable carrier and/or excipient, such that the [Cl⁻]i in a central nervous system cell is reduced in the subject.

66. The method of claim 65, wherein [Cl⁻]i levels are reduced at least about 10%, or at least about 20%, or at least about 30%, or at least about 40%, or at least about 50%, or at least about 60%, or at least about 70%, or at least about 80%, or at least about 90% compared to pre-treatment baseline.

67. A method of increasing chloride ion efflux in a central nervous system cell in a subject in need thereof, the method comprising administering to the subject a therapeutically effective amount of a GSK3β inhibitor, or a pharmaceutical composition comprising a GSK3β inhibitor and a pharmaceutically acceptable carrier and/or excipient, such that the chloride ion efflux is increased in the subject.

68. The method of claim 67, wherein chloride ion efflux levels are increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline.

69. A method of increasing synaptophysin expression in a central nervous system cell in a subject in need thereof, the method comprising administering to the subject a therapeutically effective amount of a GSK3β inhibitor, or a pharmaceutical composition comprising a GSK3β inhibitor and a pharmaceutically acceptable carrier and/or excipient, such that the synaptophysin expression is increased in the subject.

70. The method of claim 69, wherein synaptophysin expression is increased at least about 10%, or at least about 20%, or at least about 30%, or at least about 40%, or at least about 50%, or at least about 60%, or at least about 70%, or at least about 80%, or at least about 90%, or at least about 100%, or at least about 110%, or at least about 120%, or at least about 130%, or at least about 140%, or at least about 150%, or at least about 160%, or at least about 170%, or at least about 180%, or at least about 190%, about 200%, or more than 200% compared to pre-treatment baseline.

71. The method of claim 70, wherein synaptophysin expression is increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

72. A method of increasing KCC2 expression in a subject in need thereof, the method comprising administering to the subject a therapeutically effective amount of a GSK3p inhibitor, or a pharmaceutical composition comprising a GSK3β inhibitor and a pharmaceutically acceptable carrier and/or excipient, such that KCC2 expression is increased in the subject.

73. The method of claim 70, wherein KCC2 expression is increased at least about 10%, or at least about 20%, or at least about 30%, or at least about 40%, or at least about 50%, or at least about 60%, or at least about 70%, or at least about 80%, or at least about 90%, or at least about 100%, or at least about 125%, or at least about 150%, or at least about 175%, about 200%, or more than 200% compared to pre-treatment baseline.

74. The method of claim 73, wherein KCC2mRNA levels are increased at least about 30%, or at least about 40%, or at least about 50%, or at least about 60%, or at least about 70%, or at least about 80%, or at least about 90%, or at least about 100%, or at least about 125%, or at least about 150% compared to pre-treatment baseline.

75. The method as in any one of claims 60-74, wherein the GSK3β inhibitor is selected from the group consisting of CHIR-99021 (CT99021) HCl, SB-216763, CHIR-98014, TWS119, Tideglusib, SB-415286, BIO (6-bromoindirubin-3′-oxime), kenpaullone, CHIR-99021 (CT99021), AZD2858, AZD1080, AR-A014418, TDZD-8, LY2090314, IM-12, BIO-acetoxime, Indirubin, 5-Bromoindole, 2-D08, Bilinin, 1-Azakenpaullone, lithium chloride, lithium carbonate, lithium citrate, lithium orotate, lithium bromide, lithium fluoride, lithium iodide, lithium acetate, lithium hydroxide, lithium aluminum hydride, lithium perchlorate, lithium nitrate, lithium diisopropylamide, lithium borohydride, lithium oxide, lithium sulfate, lithium hexafluorophosphate, lithium tetroxide, lithium sulfide, lithium hydride, lithium amide, lithium lactate, lithium tetrafluoroborate, lithium dimethylamide, lithium phosphate, lithium peroxide, lithium manganese oxide, lithium methoxide, lithium metaborate, lithium stearate, or another lithium salt that comprises cationic lithium and any combinations thereof.

76. The method of claim 75, wherein the GSK3β inhibitor is selected from the group consisting of kenpaullone (9-bromo-7,12-dihydro-indolo [3,2-d] [1]benzazepin-6(5H)-one), NSC180515 (2-Acetyl-2,3,4,5-tetrahydrooxonine-6,9-dione), NSC79456 (n-(2,4-Dimethylphenyl)-2-hydroxy-3-nitrobenzamide), or NSC33006 (N-[4-(1,3-benzothiazol-2-yl)phenyl]acetamide).

77. The method of claim 76, wherein the GSK3β inhibitor is kenpaullone (9-bromo-7,12-dihydro-indolo [3,2-d] [1]benzazepin-6(5H)-one).

78. The method as in any of claims 60-77, the method further comprising administering to the subject a therapeutically effective amount of at least one additional compound.

79. The method according to claim 78, wherein the additional compound is selected from the group consisting of a composition according to any one of claims 35-38, an anti-analgesic, a muscle relaxant, an anti-anxiety drug, an antidepressant, an anticonvulsant, and combinations thereof.

80. The method according to claim 43, wherein the additional compound is selected from the group consisting of a composition according to any one of claims 33-36, a corticosteroid, a counterirritant, an antihistamine, and a local anesthetic and combinations thereof.

81. The method of any one of claims 44-50 or 65-71, wherein the central nervous system cell is selected from the group consisting of neurons, oligodendrocytes, astrocytes, brain parenchyma cells, and Purkinje cells.

82. The method of claim 81, wherein the central nervous system cell is a neuron.

83. The method of any one of claims 39-59, wherein the composition is administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually.

84. The method of claim 83, wherein the composition is administered intrathecally, intra-cerebroventricularly, intra-cerebrally, perispinally, intra-spinally.

85. The method of claim 84, wherein the composition is administered intrathecally.

86. The method of any one of claims 60-80, wherein the composition is administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually.

87. The method of claim 86, wherein the composition is administered topically, intravascularly, intravenously, orally, enterally, transdermally, buccally, sublingually, parenterally, subcutaneously, intramuscularly, intravenously, intradermally, or transmucosally.

88. A method for delivering a transgene to central nervous system tissue in a subject, the method comprising: administering an effective amount of a rAAV comprising a promoter operably linked with a transgene to central nervous system (CNS) tissue by intrathecal administration, wherein the rAAV infects cells of the CNS of the subject, wherein the transgene encodes a polypeptide any one of SEQ ID NO:21, SEQ ID NO:23, SEQ ID NO:25, SEQ ID NO:27, SEQ ID NO:29, SEQ ID NO:31, or SEQ ID NO:33, wherein the polypeptide reduces pathologic pain or itch.

89. The method of claim 88, wherein the intrathecal administration is in the lumbar region of the subject.

90. The method of claim 88, wherein the intrathecal administration is in the cervical region of the subject.

91. The method of claim 88, wherein the intrathecal administration is in the thoracic region of the subject.

92. The method of claim 88, wherein cells of central nervous system tissue are selected from the group consisting of oligodendrocytes, astrocytes, neurons, brain parenchyma cells, and/or Purkinje cells.

93. The method of claim 92, wherein the cells of central nervous system tissue are neurons.


SEQUENCE LISTING (33 sequences)

SEQ ID NO: 1 - DNA, 35 bp, Artificial Sequence
  Synthetic oligonucleotide
gcgccccagggcggtgcacccaccaagctgcagcg

SEQ ID NO: 2 - DNA, 35 bp, Artificial Sequence
  Synthetic oligonucleotide
cgctgcagcttggtgggtgcaccgccctggggcgc

SEQ ID NO: 3 - DNA, 38 bp, Artificial Sequence
  Synthetic oligonucleotide
gggttctagactgaaactagtgactcattggctttgtg

SEQ ID NO: 4 - DNA, 38 bp, Artificial Sequence
  Synthetic oligonucleotide
cacaaagccaatgagtcactagtttcagtctagaaccc

SEQ ID NO: 5 - DNA, 38 bp, Artificial Sequence
  Synthetic oligonucleotide
gctcaacaacctgacgactagtgaggacggcgatgggg

SEQ ID NO: 6 - DNA, 38 bp, Artificial Sequence
  Synthetic oligonucleotide
ccccatcgccgtcctcactagtcgtcaggttgttgagc

SEQ ID NO: 7 - DNA, 40 bp, Artificial Sequence
  Synthetic oligonucleotide
caaatcccttagaagcaactagtcgtccatcgaagaagac

SEQ ID NO: 8 - DNA, 40 bp, Artificial Sequence
  Synthetic oligonucleotide
gtcttcttcgatggacgactagttgcttctaagggatttg

SEQ ID NO: 9 - DNA, 20 bp, Artificial Sequence
  Synthetic oligonucleotide
ctgacggactgcgaggacgg

SEQ ID NO: 10 - DNA, 24 bp, Artificial Sequence
  Synthetic oligonucleotide
ggctggtgtccatctcctcctcaa

SEQ ID NO: 11 - DNA, 23 bp, Artificial Sequence
  Synthetic oligonucleotide
cctgccttttcgtctctagccgc

SEQ ID NO: 12 - DNA, 24 bp, Artificial Sequence
  Synthetic oligonucleotide
gctgatgacctcccagaacttggc

SEQ ID NO: 13 - DNA, 24 bp, Artificial Sequence
  Synthetic oligonucleotide
agctcatcccatactcaaaccctg

SEQ ID NO: 14 - DNA, 26 bp, Artificial Sequence
  Synthetic oligonucleotide
gcatcttggagatctaaactgctagc

SEQ ID NO: 15 - DNA, 24 bp, Artificial Sequence
  Synthetic oligonucleotide
tgcatacgggatgaggtgagcagc

SEQ ID NO: 16 - DNA, 24 bp, Artificial Sequence
  Synthetic oligonucleotide
cagaaccgtggacagcgcctagcg

SEQ ID NO: 17 - DNA, 25 bp, Artificial Sequence
  Synthetic oligonucleotide
ctgagctgtatatcacacggtctgc

SEQ ID NO: 18 - DNA, 25 bp, Artificial Sequence
  Synthetic oligonucleotide
tacgctacccagctgtctctgattc

SEQ ID NO: 19 - protein, 18 aa, Artificial Sequence
  Synthetic peptide
GGSPLTTTQGGSPTKLQR

SEQ ID NO: 20 - DNA, 5888 bp, Homo sapiens
gcacccggggccggtttcggcggcggggcggacgctccctcactagccgggcgcccgggccggccgccgaggtttaggtggaggctgaggctgccgcgcgccgcgggaggagcctcgccctcggcggctgaggcggcggcggcacaggtggcgcgggccgcgcgcggggcgcagctcgggagcgctcggcgccgggcgccgcggcggccccaggctcgcgcccgcggcgacagccggctgagcggagctgcgggcgcggcggctgctctctcagcccggccggctggcggcggcgcccggagccccgaggagcccgcaggagcccgtggagcccgcggagccgggcagggggcgctgcgctcgccgagctcggaggggccgccgggccgggcgctgcgcactcgcgtcgggagccgcctctcgcccgcggcgctcgcccctgccgcccgccagcatcccttgtcccgcggccgcgctcagacaacaaaagcggaagatgctgcagttgggcaaggtcaggaccttgccctgaagccgggcggcgccgcgcacgcctcttcccggactgaggagctgtcgccggcggagggtgcatgtttgcgaggaagccgccgggcgccgcgcctttgggagctatgcctgttccagaccagccttcatcagcctcagagaagacgagttccctgagccccggcttaaacacctccaacggggatggctctgaaacagaaaccacctctgccatcctcgcctcagtcaaagaacaggaattacagtttgaaaggctgacccgagagctggaggctgaacggcagatcgtagccagccagctggagcgatgcaagctcggatccgagactggcagcatgagcagcatgagttcagcagaagagcagtttcagtggcagtcacaagatggtcaaaaagatatcgaagatgagcttacaacaggtctcgagctggtggactcctgtattaggtcactacaggaatcaggaatacttgacccacaggattattctacaggtgaaaggcccagcctgctctcccagagtgcacttcagctcaattccaaacctgaagggtctttccagtatccggccagctaccatagcaaccagaccctggccctgggggaaaccaccccttcacagctcccggcccgaggcacacaagcccgagctacgggccagagcttcagccagggcacgaccagccgcgccggccacctggcggggcccgagcccgcgccgccgccgccgccgccgccgcgggagccgttcgcgcccagcctgggcagcgccttccacctgcccgacgcgccgcccgccgccgccgccgccgcgctctactactccagctccacgctgcccgcgccgccgcgcgggggctccccgctggccgcgccccagggcggttcgcccaccaagctgcagcgcggcggctcggcccccgagggcgccacctacgccgcgccgcgcggctcctcgcccaagcagtcgcccagccgcctggccaagtcctacagcaccagctcgcccatcaacatcgtcgtgtcctcggccggcctgtccccgatccgcgtgacctcgccccccaccgtgcagtccaccatctcctcctcgcccatccaccagctgagctccaccatcggcacgtacgccaccctgtcgcccaccaagcgcctggtccacgcgtccgagcagtacagcaagcactcgcaggagctgtatgccacggccaccctccagaggccgggcagcctggcagctggttcccgagcctcatacagcagccagcatgggcacctgggcccagagttgcgggccctgcagtccccagaacaccacatagatcccatctatgaagaccgcgtctatcagaagccccctatgaggagtctcagccagagccagggggaccctctgccgccagcacacaccggcacctaccgcacgagcacagccccatcttcccctggtgtcgactccgtccccttgcagcgcacaggcagccagcacggcccacagaatgccgccgcggccaccttccagagggccagctatgccgccggcccagcctccaattacgcggacccctaccgacagctgcagtattgtccctctgttgagtctccatacagcaaatccggccctgctctcccgcctgaaggcaccttggccaggtccccgtccattgatagcattcagaaagatcccagagaatttggatggagagacccggaactgccggaagtgattcagatgttgcagcaccagtttccctcggtccagtctaacgcggcagcctacttgcaacacctctgttttggagacaacaaaattaaagccgagataaggagacaaggaggcatccagctcctggtggacctgttggatcatcggatgaccgaagtccaccgtagtgcctgtggagctctgagaaacctggtgtatgggaaggccaacgatgataacaaaattgccctgaaaaactgtggtggcatcccagcactggtgaggttactccgcaagacgactgacctggagatccgggagctggtcacaggagtcctttggaacctctcctcatgcgatgcactcaaaatgccaatcatccaggatgccctagcagtactgaccaacgcggtgattatcccccactcaggctgggaaaattcgcctcttcaggatgatcggaaaatacagctgcattcatcacaggtgctgcgtaacgccaccgggtgcctaaggaatgttagttcggccggagaggaggcccgcagaaggatgagagagtgtgatgggcttacggatgccttgctgtacgtgatccagtctgcgctggggagcagtgagatcgatagcaagaccgttgaaaactgtgtgtgcattttaaggaacctctcgtaccggctggcggcagaaacgtctcagggacagcacatgggcacggacgagctggacgggctactctgtggcgaggccaatggcaaggatgctgagagctctgggtgctggggcaagaagaagaagaaaaagaaatcccaagatcagtgggatggagtaggacctcttccagactgtgctgaaccaccaaaagggatccagatgctgtggcacccatcaatagtcaaaccctacctcacactgctctctgagtgctcaaatccagacacgctggaaggggcggcaggcgccctgcagaacttggctgcagggagctggaagtggtcagtatatatccgagccgctgtccgaaaagagaaaggcctgcccatcctcgtggagctgctccgaatagacaatgaccgtgtggtgtgcgcggtggccactgcgctgcggaacatggccttggacgtcagaaataaggagctcatcggcaaatacgccatgcgagacctagtccacaggcttccaggagggaacaacagcaacaacactgcaagcaaggccatgtcggatgacacagtgacagctgtctgctgcacactgcacgaagtgattaccaagaacatggagaacgccaaggccttacgggatgccggtggcatcgagaagttggtcggcatctccaaaagcaaaggagataaacactctccaaaagtggtcaaggctgcatctcaggtcctcaacagcatgtggcagtaccgagatctgaggagtctctacaaaaaggatggatggtcacaataccactttgtagcctcgtcttcaaccatcgagagggaccggcaaaggccctactcctcctcccgcacgccctccatctcccctgtgcgcgtgtctcccaacaaccgctcagcaagtgccccagcttcacctcgggaaatgatcagcctcaaagaaaggaaaacagactacgagtgcaccggcagcaacgccacctaccacggagctaaaggcgaacacacttccaggaaagatgccatgacagctcaaaacactggaatttcaactttgtataggaattcttatggtgcgcccgctgaagacatcaaacacaaccaggtttcagcacagccagtcccacaggagcccagcagaaaagattacgagacctaccagccatttcagaattccacaagaaattacgatgagtccttcttcgaggaccaggtccaccatcgccctcccgccagcgagtacaccatgcacctgggtctcaagtccaccggcaactacgttgacttctactcagctgcccgtccctacagtgaactgaactatgaaacgagccactacccggcctcccccgactcctgggtgtgaggagcagggcacaggcgctccgggaacagtgcatgtgcatgcataccacaagacatttctttctgttttgtttttttctcctgcaaatttagtttgttaaagcctgttccataggaaggctgtgataaccagtaaggaaatattaagagctattttagaaagctaaatgaatcgcaagttaacttggaaatcagtagaaagctaaagtgatcctaaatatgacagtgggcagcacctttctagcgtgagctgtagagtaacgagaagtgctttatactgaacgtggttgatgggaggagagacgaggcattcgggccggtggggcgtaagggttatcgttaagcacaagacacagaatagtttacacactgtgtgggggacggcttctcacgctttgtttactctcttcatccgttgtgactctaggcttcaggttgcattggggttcctctgtacagcaagatgtttcttgccttttgttaatgcattgttgtaaagtatttgatgtacattacagattaaagaagaaaagcgcgttgtgtatattacaccaatgccgccgtgtttcctcatctatggttctaaatattgcttcaatttcaaacttttgaaagatgtatggatttccagtttttctttactttctcccagtatgttttaacaaaaaaaaaaaaaagcaggaaaaaaggaatatttagcagtattgttcgttctgatatgtgaatttgtttgtgacaactaaacaaggcattcagcagtttctgacaattaacatacatcattccacactccttgtcaacaaagtgctttttcactgcctaaaattttagatgtagatatttgaaatagattttttcatttataccagttttctttatgatgatacagtgttaaaagaaaataaattacaattgatctgtcatccatatttgctaagaatgtctatttccaagaacctatatcttacaaaatacacattcttgcttgtgtgtctgtgtgtgtgggtgtatgtatgtccacgtactagaaaaattacctgtgtgagattttattttgacagaagctatttcatttgctgttcatcttgtacaaactttgtttttggttttttagtataaatgtacatcagtttgttccttttgtactgtatctattcttctgaccatctagtgactcaggatattaggccagttgaagtggagttataggattttcacacattcacgcatacttggatatcaaccctctctacaggtccgtttccttacacattttatcccctcagaacacgataaaccatggccaattcagtttcactttggggccaattacttgaagtatccacagaaaatgtaaactgctcgccttattcccccaaactttttgttcatgtggcagggagcagtacacaactatggaatagattattaaaactgaacttgaagctaacgttagaatagtggttaacagagctaagaaataaggctaaccatcgaaaagaacatgggccgcgggtaaactagctttgctctttagatgcaagtggctgaggccaataggtgcctagtaaatgttaactgttcttggaaaaaataaaaacatcagtaacaaaagaaa

SEQ ID NO: 21 - protein, 1225 aa, Homo sapiens
MFARKPPGAAPLGAMPVPDQPSSASEKTSSLSPGLNTSNGDGSETETTSAILASVKEQELQFERLTRELEAERQIVASQLERCKLGSETGSMSSMSSAEEQFQWQSQDGQKDIEDELTTGLELVDSCIRSLQESGILDPQDYSTGERPSLLSQSALQLNSKPEGSFQYPASYHSNQTLALGETTPSQLPARGTQARATGQSFSQGTTSRAGHLAGPEPAPPPPPPPREPFAPSLGSAFHLPDAPPAAAAAALYYSSSTLPAPPRGGSPLAAPQGGSPTKLQRGGSAPEGATYAAPRGSSPKQSPSRLAKSYSTSSPINIVVSSAGLSPIRVTSPPTVQSTISSSPIHQLSSTIGTYATLSPTKRLVHASEQYSKHSQELYATATLQRPGSLAAGSRASYSSQHGHLGPELRALQSPEHHIDPIYEDRVYQKPPMRSLSQSQGDPLPPAHTGTYRTSTAPSSPGVDSVPLQRTGSQHGPQNAAAATFQRASYAAGPASNYADPYRQLQYCPSVESPYSKSGPALPPEGTLARSPSIDSIQKDPREFGWRDPELPEVIQMLQHQFPSVQSNAAAYLQHLCFGDNKIKAEIRRQGGIQLLVDLLDHRMTEVHRSACGALRNLVYGKANDDNKIALKNCGGIPALVRLLRKTTDLEIRELVTGVLWNLSSCDALKMPIIQDALAVLTNAVIIPHSGWENSPLQDDRKIQLHSSQVLRNATGCLRNVSSAGEEARRRMRECDGLTDALLYVIQSALGSSEIDSKTVENCVCILRNLSYRLAAETSQGQHMGTDELDGLLCGEANGKDAESSGCWGKKKKKKKSQDQWDGVGPLPDCAEPPKGIQMLWHPSIVKPYLTLLSECSNPDTLEGAAGALQNLAAGSWKWSVYIRAAVRKEKGLPILVELLRIDNDRVVCAVATALRNMALDVRNKELIGKYAMRDLVHRLPGGNNSNNTASKAMSDDTVTAVCCTLHEVITKNMENAKALRDAGGIEKLVGISKSKGDKHSPKVVKAASQVLNSMWQYRDLRSLYKKDGWSQYHFVASSSTIERDRQRPYSSSRTPSISPVRVSPNNRSASAPASPREMISLKERKTDYECTGSNATYHGAKGEHTSRKDAMTAQNTGISTLYRNSYGAPAEDIKHNQVSAQPVPQEPSRKDYETYQPFQNSTRNYDESFFEDQVHHRPPASEYTMHLGLKSTGNYVDFYSAARPYSELNYETSHYPASPDSWV

SEQ ID NO: 22 - DNA, 5289 bp, Homo sapiens
gttccttttagccgtggtgcagggagggaggagggaggtgggtaggatgccaggaaccgcctgctccacagcctggggaaggagggaagagtaacccttcatctttgggactagtcgtcttgtttgcctagactacagttttcaagtatttaaggaccatttatttcaaaaggaattacagtttgaaaggctgacccgagagctggaggctgaacggcagatcgtagccagccagctggagcgatgcaagctcggatccgagactggcagcatgagcagcatgagttcagcagaagagcagtttcagtggcagtcacaagatggtcaaaaagatatcgaagatgagcttacaacaggtctcgagctggtggactcctgtattaggtcactacaggaatcaggaatacttgacccacaggattattctacaggtgaaaggcccagcctgctctcccagagtgcacttcagctcaattccaaacctgaagggtctttccagtatccggccagctaccatagcaaccagaccctggccctgggggaaaccaccccttcacagctcccggcccgaggcacacaagcccgagctacgggccagagcttcagccagggcacgaccagccgcgccggccacctggcggggcccgagcccgcgccgccgccgccgccgccgccgcgggagccgttcgcgcccagcctgggcagcgccttccacctgcccgacgcgccgcccgccgccgccgccgccgcgctctactactccagctccacgctgcccgcgccgccgcgcgggggctccccgctggccgcgccccagggcggttcgcccaccaagctgcagcgcggcggctcggcccccgagggcgccacctacgccgcgccgcgcggctcctcgcccaagcagtcgcccagccgcctggccaagtcctacagcaccagctcgcccatcaacatcgtcgtgtcctcggccggcctgtccccgatccgcgtgacctcgccccccaccgtgcagtccaccatctcctcctcgcccatccaccagctgagctccaccatcggcacgtacgccaccctgtcgcccaccaagcgcctggtccacgcgtccgagcagtacagcaagcactcgcaggagctgtatgccacggccaccctccagaggccgggcagcctggcagctggttcccgagcctcatacagcagccagcatgggcacctgggcccagagttgcgggccctgcagtccccagaacaccacatagatcccatctatgaagaccgcgtctatcagaagccccctatgaggagtctcagccagagccagggggaccctctgccgccagcacacaccggcacctaccgcacgagcacagccccatcttcccctggtgtcgactccgtccccttgcagcgcacaggcagccagcacggcccacagaatgccgccgcggccaccttccagagggccagctatgccgccggcccagcctccaattacgcggacccctaccgacagctgcagtattgtccctctgttgagtctccatacagcaaatccggccctgctctcccgcctgaaggcaccttggccaggtccccgtccattgatagcattcagaaagatcccagagaatttggatggagagacccggaactgccggaagtgattcagatgttgcagcaccagtttccctcggtccagtctaacgcggcagcctacttgcaacacctctgttttggagacaacaaaattaaagccgagataaggagacaaggaggcatccagctcctggtggacctgttggatcatcggatgaccgaagtccaccgtagtgcctgtggagctctgagaaacctggtgtatgggaaggccaacgatgataacaaaattgccctgaaaaactgtggtggcatcccagcactggtgaggttactccgcaagacgactgacctggagatccgggagctggtcacaggagtcctttggaacctctcctcatgcgatgcactcaaaatgccaatcatccaggatgccctagcagtactgaccaacgcggtgattatcccccactcaggctgggaaaattcgcctcttcaggatgatcggaaaatacagctgcattcatcacaggtgctgcgtaacgccaccgggtgcctaaggaatgttagttcggccggagaggaggcccgcagaaggatgagagagtgtgatgggcttacggatgccttgctgtacgtgatccagtctgcgctggggagcagtgagatcgatagcaagaccgttgaaaactgtgtgtgcattttaaggaacctctcgtaccggctggcggcagaaacgtctcagggacagcacatgggcacggacgagctggacgggctactctgtggcgaggccaatggcaaggatgctgagagctctgggtgctggggcaagaagaagaagaaaaagaaatcccaagatcagtgggatggagtaggacctcttccagactgtgctgaaccaccaaaagggatccagatgctgtggcacccatcaatagtcaaaccctacctcacactgctctctgagtgctcaaatccagacacgctggaaggggcggcaggcgccctgcagaacttggctgcagggagctggaagtggtcagtatatatccgagccgctgtccgaaaagagaaaggcctgcccatcctcgtggagctgctccgaatagacaatgaccgtgtggtgtgcgcggtggccactgcgctgcggaacatggccttggacgtcagaaataaggagctcatcggcaaatacgccatgcgagacctagtccacaggcttccaggagggaacaacagcaacaacactgcaagcaaggccatgtcggatgacacagtgacagctgtctgctgcacactgcacgaagtgattaccaagaacatggagaacgccaaggccttacgggatgccggtggcatcgagaagttggtcggcatctccaaaagcaaaggagataaacactctccaaaagtggtcaaggctgcatctcaggtcctcaacagcatgtggcagtaccgagatctgaggagtctctacaaaaaggatggatggtcacaataccactttgtagcctcgtcttcaaccatcgagagggaccggcaaaggccctactcctcctcccgcacgccctccatctcccctgtgcgcgtgtctcccaacaaccgctcagcaagtgccccagcttcacctcgggaaatgatcagcctcaaagaaaggaaaacagactacgagtgcaccggcagcaacgccacctaccacggagctaaaggcgaacacacttccaggaaagatgccatgacagctcaaaacactggaatttcaactttgtataggaattcttatggtgcgcccgctgaagacatcaaacacaaccaggtttcagcacagccagtcccacaggagcccagcagaaaagattacgagacctaccagccatttcagaattccacaagaaattacgatgagtccttcttcgaggaccaggtccaccatcgccctcccgccagcgagtacaccatgcacctgggtctcaagtccaccggcaactacgttgacttctactcagctgcccgtccctacagtgaactgaactatgaaacgagccactacccggcctcccccgactcctgggtgtgaggagcagggcacaggcgctccgggaacagtgcatgtgcatgcataccacaagacatttctttctgttttgtttttttctcctgcaaatttagtttgttaaagcctgttccataggaaggctgtgataaccagtaaggaaatattaagagctattttagaaagctaaatgaatcgcaagttaacttggaaatcagtagaaagctaaagtgatcctaaatatgacagtgggcagcacctttctagcgtgagctgtagagtaacgagaagtgctttatactgaacgtggttgatgggaggagagacgaggcattcgggccggtggggcgtaagggttatcgttaagcacaagacacagaatagtttacacactgtgtgggggacggcttctcacgctttgtttactctcttcatccgttgtgactctaggcttcaggttgcattggggttcctctgtacagcaagatgtttcttgccttttgttaatgcattgttgtaaagtatttgatgtacattacagattaaagaagaaaagcgcgttgtgtatattacaccaatgccgccgtgtttcctcatctatggttctaaatattgcttcaatttcaaacttttgaaagatgtatggatttccagtttttctttactttctcccagtatgttttaacaaaaaaaaaaaaaagcaggaaaaaaggaatatttagcagtattgttcgttctgatatgtgaatttgtttgtgacaactaaacaaggcattcagcagtttctgacaattaacatacatcattccacactccttgtcaacaaagtgctttttcactgcctaaaattttagatgtagatatttgaaatagattttttcatttataccagttttctttatgatgatacagtgttaaaagaaaataaattacaattgatctgtcatccatatttgctaagaatgtctatttccaagaacctatatcttacaaaatacacattcttgcttgtgtgtctgtgtgtgtgggtgtatgtatgtccacgtactagaaaaattacctgtgtgagattttattttgacagaagctatttcatttgctgttcatcttgtacaaactttgtttttggttttttagtataaatgtacatcagtttgttccttttgtactgtatctattcttctgaccatctagtgactcaggatattaggccagttgaagtggagttataggattttcacacattcacgcatacttggatatcaaccctctctacaggtccgtttccttacacattttatcccctcagaacacgataaaccatggccaattcagtttcactttggggccaattacttgaagtatccacagaaaatgtaaactgctcgccttattcccccaaactttttgttcatgtggcagggagcagtacacaactatggaatagattattaaaactgaacttgaagctaacgttagaatagtggttaacagagctaagaaataaggctaaccatcgaaaagaacatgggccgcgggtaaactagctttgctctttagatgcaagtggctgaggccaataggtgcctagtaaatgttaactgttcttggaaaaaataaaaacatcagtaacaaaa

SEQ ID NO: 23 - protein, 1134 aa, Homo sapiens
MSSMSSAEEQFQWQSQDGQKDIEDELTTGLELVDSCIRSLQESGILDPQDYSTGERPSLLSQSALQLNSKPEGSFQYPASYHSNQTLALGETTPSQLPARGTQARATGQSFSQGTTSRAGHLAGPEPAPPPPPPPREPFAPSLGSAFHLPDAPPAAAAAALYYSSSTLPAPPRGGSPLAAPQGGSPTKLQRGGSAPEGATYAAPRGSSPKQSPSRLAKSYSTSSPINIVVSSAGLSPIRVTSPPTVQSTISSSPIHQLSSTIGTYATLSPTKRLVHASEQYSKHSQELYATATLQRPGSLAAGSRASYSSQHGHLGPELRALQSPEHHIDPIYEDRVYQKPPMRSLSQSQGDPLPPAHTGTYRTSTAPSSPGVDSVPLQRTGSQHGPQNAAAATFQRASYAAGPASNYADPYRQLQYCPSVESPYSKSGPALPPEGTLARSPSIDSIQKDPREFGWRDPELPEVIQMLQHQFPSVQSNAAAYLQHLCFGDNKIKAEIRRQGGIQLLVDLLDHRMTEVHRSACGALRNLVYGKANDDNKIALKNCGGIPALVRLLRKTTDLEIRELVTGVLWNLSSCDALKMPIIQDALAVLTNAVIIPHSGWENSPLQDDRKIQLHSSQVLRNATGCLRNVSSAGEEARRRMRECDGLTDALLYVIQSALGSSEIDSKTVENCVCILRNLSYRLAAETSQGQHMGTDELDGLLCGEANGKDAESSGCWGKKKKKKKSQDQWDGVGPLPDCAEPPKGIQMLWHPSIVKPYLTLLSECSNPDTLEGAAGALQNLAAGSWKWSVYIRAAVRKEKGLPILVELLRIDNDRVVCAVATALRNMALDVRNKELIGKYAMRDLVHRLPGGNNSNNTASKAMSDDTVTAVCCTLHEVITKNMENAKALRDAGGIEKLVGISKSKGDKHSPKVVKAASQVLNSMWQYRDLRSLYKKDGWSQYHFVASSSTIERDRQRPYSSSRTPSISPVRVSPNNRSASAPASPREMISLKERKTDYECTGSNATYHGAKGEHTSRKDAMTAQNTGISTLYRNSYGAPAEDIKHNQVSAQPVPQEPSRKDYETYQPFQNSTRNYDESFFEDQVHHRPPASEYTMHLGLKSTGNYVDFYSAARPYSELNYETSHYPASPDSWV

SEQ ID NO: 24 - DNA, 4551 bp, Homo sapiens
gttccttttagccgtggtgcagggagggaggagggaggtgggtaggatgccaggaaccgcctgctccacagcctggggaaggagggaagagtaacccttcatctttgggactagtcgtcttgtttgcctagactacagttttcaagtatttaaggaccatttatttcaaaaggaattacagtttgaaaggctgacccgagagctggaggctgaacggcagatcgtagccagccagctggagcgatgcaagctcggatccgagactggcagcatgagcagcatgagttcagcagaagagcagtttcagtggcagtcacaagatggtcaaaaagatatcgaagatgagcttacaacaggtctcgagctggtggactcctgtattaggtcactacaggaatcaggaatacttgacccacaggattattctacaggtgaaactggttcccgagcctcatacagcagccagcatgggcacctgggcccagagttgcgggccctgcagtccccagaacaccacatagatcccatctatgaagaccgcgtctatcagaagccccctatgaggagtctcagccagagccagggggaccctctgccgccagcacacaccggcacctaccgcacgagcacagccccatcttcccctggtgtcgactccgtccccttgcagcgcacaggcagccagcacggcccacagaatgccgccgcggccaccttccagagggccagctatgccgccggcccagcctccaattacgcggacccctaccgacagctgcagtattgtccctctgttgagtctccatacagcaaatccggccctgctctcccgcctgaaggcaccttggccaggtccccgtccattgatagcattcagaaagatcccagagaatttggatggagagacccggaactgccggaagtgattcagatgttgcagcaccagtttccctcggtccagtctaacgcggcagcctacttgcaacacctctgttttggagacaacaaaattaaagccgagataaggagacaaggaggcatccagctcctggtggacctgttggatcatcggatgaccgaagtccaccgtagtgcctgtggagctctgagaaacctggtgtatgggaaggccaacgatgataacaaaattgccctgaaaaactgtggtggcatcccagcactggtgaggttactccgcaagacgactgacctggagatccgggagctggtcacaggagtcctttggaacctctcctcatgcgatgcactcaaaatgccaatcatccaggatgccctagcagtactgaccaacgcggtgattatcccccactcaggctgggaaaattcgcctcttcaggatgatcggaaaatacagctgcattcatcacaggtgctgcgtaacgccaccgggtgcctaaggaatgttagttcggccggagaggaggcccgcagaaggatgagagagtgtgatgggcttacggatgccttgctgtacgtgatccagtctgcgctggggagcagtgagatcgatagcaagaccgttgaaaactgtgtgtgcattttaaggaacctctcgtaccggctggcggcagaaacgtctcagggacagcacatgggcacggacgagctggacgggctactctgtggcgaggccaatggcaaggatgctgagagctctgggtgctggggcaagaagaagaagaaaaagaaatcccaagatcagtgggatggagtaggacctcttccagactgtgctgaaccaccaaaagggatccagatgctgtggcacccatcaatagtcaaaccctacctcacactgctctctgagtgctcaaatccagacacgctggaaggggcggcaggcgccctgcagaacttggctgcagggagctggaagtggtcagtatatatccgagccgctgtccgaaaagagaaaggcctgcccatcctcgtggagctgctccgaatagacaatgaccgtgtggtgtgcgcggtggccactgcgctgcggaacatggccttggacgtcagaaataaggagctcatcggcaaatacgccatgcgagacctagtccacaggcttccaggagggaacaacagcaacaacactgcaagcaaggccatgtcggatgacacagtgacagctgtctgctgcacactgcacgaagtgattaccaagaacatggagaacgccaaggccttacgggatgccggtggcatcgagaagttggtcggcatctccaaaagcaaaggagataaacactctccaaaagtggtcaaggctgcatctcaggtcctcaacagcatgtggcagtaccgagatctgaggagtctctacaaaaaggatggatggtcacaataccactttgtagcctcgtcttcaaccatcgagagggaccggcaaaggccctactcctcctcccgcacgccctccatctcccctgtgcgcgtgtctcccaacaaccgctcagcaagtgccccagcttcacctcgggaaatgatcagcctcaaagaaaggaaaacagactacgagtgcaccggcagcaacgccacctaccacggagctaaaggcgaacacacttccaggaaagatgccatgacagctcaaaacactggaatttcaactttgtataggaattcttatggtgcgcccgctgaagacatcaaacacaaccaggtttcagcacagccagtcccacaggagcccagcagaaaagattacgagacctaccagccatttcagaattccacaagaaattacgatgagtccttcttcgaggaccaggtccaccatcgccctcccgccagcgagtacaccatgcacctgggtctcaagtccaccggcaactacgttgacttctactcagctgcccgtccctacagtgaactgaactatgaaacgagccactacccggcctcccccgactcctgggtgtgaggagcagggcacaggcgctccgggaacagtgcatgtgcatgcataccacaagacatttctttctgttttgtttttttctcctgcaaatttagtttgttaaagcctgttccataggaaggctgtgataaccagtaaggaaatattaagagctattttagaaagctaaatgaatcgcaagttaacttggaaatcagtagaaagctaaagtgatcctaaatatgacagtgggcagcacctttctagcgtgagctgtagagtaacgagaagtgctttatactgaacgtggttgatgggaggagagacgaggcattcgggccggtggggcgtaagggttatcgttaagcacaagacacagaatagtttacacactgtgtgggggacggcttctcacgctttgtttactctcttcatccgttgtgactctaggcttcaggttgcattggggttcctctgtacagcaagatgtttcttgccttttgttaatgcattgttgtaaagtatttgatgtacattacagattaaagaagaaaagcgcgttgtgtatattacaccaatgccgccgtgtttcctcatctatggttctaaatattgcttcaatttcaaacttttgaaagatgtatggatttccagtttttctttactttctcccagtatgttttaacaaaaaaaaaaaaaagcaggaaaaaaggaatatttagcagtattgttcgttctgatatgtgaatttgtttgtgacaactaaacaaggcattcagcagtttctgacaattaacatacatcattccacactccttgtcaacaaagtgctttttcactgcctaaaattttagatgtagatatttgaaatagattttttcatttataccagttttctttatgatgatacagtgttaaaagaaaataaattacaattgatctgtcatccatatttgctaagaatgtctatttccaagaacctatatcttacaaaatacacattcttgcttgtgtgtctgtgtgtgtgggtgtatgtatgtccacgtactagaaaaattacctgtgtgagattttattttgacagaagctatttcatttgctgttcatcttgtacaaactttgtttttggttttttagtataaatgtacatcagtttgttccttttgtactgtatctattcttctgaccatctagtgactcaggatattaggccagttgaagtggagttataggattttcacacattcacgcatacttggatatcaaccctctctacaggtccgtttccttacacattttatcccctcagaacacgataaaccatggccaattcagtttcactttggggccaattacttgaagtatccacagaaaatgtaaactgctcgccttattcccccaaactttttgttcatgtggcagggagcagtacacaactatggaatagattattaaaactgaacttgaagctaacgttagaatagtggttaacagagctaagaaataaggctaaccatcgaaaagaacatgggccgcgggtaaactagctttgctctttagatgcaagtggctgaggccaataggtgcctagtaaatgttaactgttcttggaaaaaataaaaacatcagtaacaaaa

SEQ ID NO: 25 - protein, 888 aa, Homo sapiens
MSSMSSAEEQFQWQSQDGQKDIEDELTTGLELVDSCIRSLQESGILDPQDYSTGETGSRASYSSQHGHLGPELRALQSPEHHIDPIYEDRVYQKPPMRSLSQSQGDPLPPAHTGTYRTSTAPSSPGVDSVPLQRTGSQHGPQNAAAATFQRASYAAGPASNYADPYRQLQYCPSVESPYSKSGPALPPEGTLARSPSIDSIQKDPREFGWRDPELPEVIQMLQHQFPSVQSNAAAYLQHLCFGDNKIKAEIRRQGGIQLLVDLLDHRMTEVHRSACGALRNLVYGKANDDNKIALKNCGGIPALVRLLRKTTDLEIRELVTGVLWNLSSCDALKMPIIQDALAVLTNAVIIPHSGWENSPLQDDRKIQLHSSQVLRNATGCLRNVSSAGEEARRRMRECDGLTDALLYVIQSALGSSEIDSKTVENCVCILRNLSYRLAAETSQGQHMGTDELDGLLCGEANGKDAESSGCWGKKKKKKKSQDQWDGVGPLPDCAEPPKGIQMLWHPSIVKPYLTLLSECSNPDTLEGAAGALQNLAAGSWKWSVYIRAAVRKEKGLPILVELLRIDNDRVVCAVATALRNMALDVRNKELIGKYAMRDLVHRLPGGNNSNNTASKAMSDDTVTAVCCTLHEVITKNMENAKALRDAGGIEKLVGISKSKGDKHSPKVVKAASQVLNSMWQYRDLRSLYKKDGWSQYHFVASSSTIERDRQRPYSSSRTPSISPVRVSPNNRSASAPASPREMISLKERKTDYECTGSNATYHGAKGEHTSRKDAMTAQNTGISTLYRNSYGAPAEDIKHNQVSAQPVPQEPSRKDYETYQPFQNSTRNYDESFFEDQVHHRPPASEYTMHLGLKSTGNYVDFYSAARPYSELNYETSHYPASPDSWV

SEQ ID NO: 26 - DNA, 5323 bp, Homo sapiens
gcacccggggccggtttcggcggcggggcggacgctccctcactagccgggcgcccgggccggccgccgaggtttaggtggaggctgaggctgccgcgcgccgcgggaggagcctcgccctcggcggctgaggcggcggcggcacaggtggcgcgggccgcgcgcggggcgcagctcgggagcgctcggcgccgggcgccgcggcggccccaggctcgcgcccgcggcgacagccggctgagcggagctgcgggcgcggcggctgctctctcagcccggccggctggcggcggcgcccggagccccgaggagcccgcaggagcccgtggagcccgcggagccgggcagggggcgctgcgctcgccgagctcggaggggccgccgggccgggcgctgcgcactcgcgtcgggagccgcctctcgcccgcggcgctcgcccctgccgcccgccagcatcccttgtcccgcggccgcgctcagacaacaaaagcggaagatgctgcagttgggcaaggtcaggaccttgccctgaagccgggcggcgccgcgcacgcctcttcccggactgaggagctgtcgccggcggagggtgcatgtttgcgaggaagccgccgggcgccgcgcctttgggagctatgcctgttccagaccagccttcatcagcctcagagaagacgagttccctgagccccggcttaaacacctccaacggggatggctctgaaacagaaaccacctctgccatcctcgcctcagtcaaagaacaggaattacagtttgaaaggctgacccgagagctggaggctgaacggcagatcgtagccagccagctggagcgatgcaagctcggatccgagactggcagcatgagcagcatgagttcagcagaagagcagtttcagtggcagtcacaagatggtcaaaaagatatcgaagatgagcttacaacaggtctcgagctggtggactcctgtattaggtcactacaggaatcaggaatacttgacccacaggattattctacaggtgaaaggcccagcctgctctcccagagtgcacttcagctcaattccaaacctgaagggtctttccagtatccggccagctaccatagcaaccagaccctggccctgggggaaaccaccccttcacagctcccggcccgaggcacacaagcccgagctacgggccagagcttcagccagctggttcccgagcctcatacagcagccagcatgggcacctgggcccagagttgcgggccctgcagtccccagaacaccacatagatcccatctatgaagaccgcgtctatcagaagccccctatgaggagtctcagccagagccagggggaccctctgccgccagcacacaccggcacctaccgcacgagcacagccccatcttcccctggtgtcgactccgtccccttgcagcgcacaggcagccagcacggcccacagaatgccgccgcggccaccttccagagggccagctatgccgccggcccagcctccaattacgcggacccctaccgacagctgcagtattgtccctctgttgagtctccatacagcaaatccggccctgctctcccgcctgaaggcaccttggccaggtccccgtccattgatagcattcagaaagatcccagagaatttggatggagagacccggaactgccggaagtgattcagatgttgcagcaccagtttccctcggtccagtctaacgcggcagcctacttgcaacacctctgttttggagacaacaaaattaaagccgagataaggagacaaggaggcatccagctcctggtggacctgttggatcatcggatgaccgaagtccaccgtagtgcctgtggagctctgagaaacctggtgtatgggaaggccaacgatgataacaaaattgccctgaaaaactgtggtggcatcccagcactggtgaggttactccgcaagacgactgacctggagatccgggagctggtcacaggagtcctttggaacctctcctcatgcgatgcactcaaaatgccaatcatccaggatgccctagcagtactgaccaacgcggtgattatcccccactcaggctgggaaaattcgcctcttcaggatgatcggaaaatacagctgcattcatcacaggtgctgcgtaacgccaccgggtgcctaaggaatgttagttcggccggagaggaggcccgcagaaggatgagagagtgtgatgggcttacggatgccttgctgtacgtgatccagtctgcgctggggagcagtgagatcgatagcaagaccgttgaaaactgtgtgtgcattttaaggaacctctcgtaccggctggcggcagaaacgtctcagggacagcacatgggcacggacgagctggacgggctactctgtggcgaggccaatggcaaggatgctgagagctctgggtgctggggcaagaagaagaagaaaaagaaatcccaagatcagtgggatggagtaggacctcttccagactgtgctgaaccaccaaaagggatccagatgctgtggcacccatcaatagtcaaaccctacctcacactgctctctgagtgctcaaatccagacacgctggaaggggcggcaggcgccctgcagaacttggctgcagggagctggaagtggtcagtatatatccgagccgctgtccgaaaagagaaaggcctgcccatcctcgtggagctgctccgaatagacaatgaccgtgtggtgtgcgcggtggccactgcgctgcggaacatggccttggacgtcagaaataaggagctcatcggcaaatacgccatgcgagacctagtccacaggcttccaggagggaacaacagcaacaacactgcaagcaaggccatgtcggatgacacagtgacagctgtctgctgcacactgcacgaagtgattaccaagaacatggagaacgccaaggccttacgggatgccggtggcatcgagaagttggtcggcatctccaaaagcaaaggagataaacactctccaaaagtggtcaaggctgcatctcaggtcctcaacagcatgtggcagtaccgagatctgaggagtctctacaaaaaggatggatggtcacaataccactttgtagcctcgtcttcaaccatcgagagggaccggcaaaggccctactcctcctcccgcacgccctccatctcccctgtgcgcgtgtctcccaacaaccgctcagcaagtgccccagcttcacctcgggaaatgatcagcctcaaagaaaggaaaacagactacgagtgcaccggcagcaacgccacctaccacggagctaaaggcgaacacacttccaggaaagatgccatgacagctcaaaacactggaatttcaactttgtataggaattcttatggtgcgcccgctgaagacatcaaacacaaccaggtttcagcacagccagtcccacaggagcccagcagaaaagattacgagacctaccagccatttcagaattccacaagaaattacgatgagtccttcttcgaggaccaggtccaccatcgccctcccgccagcgagtacaccatgcacctgggtctcaagtccaccggcaactacgttgacttctactcagctgcccgtccctacagtgaactgaactatgaaacgagccactacccggcctcccccgactcctgggtgtgaggagcagggcacaggcgctccgggaacagtgcatgtgcatgcataccacaagacatttctttctgttttgtttttttctcctgcaaatttagtttgttaaagcctgttccataggaaggctgtgataaccagtaaggaaatattaagagctattttagaaagctaaatgaatcgcaagttaacttggaaatcagtagaaagctaaagtgatcctaaatatgacagtgggcagcacctttctagcgtgagctgtagagtaacgagaagtgctttatactgaacgtggttgatgggaggagagacgaggcattcgggccggtggggcgtaagggttatcgttaagcacaagacacagaatagtttacacactgtgtgggggacggcttctcacgctttgtttactctcttcatccgttgtgactctaggcttcaggttgcattggggttcctctgtacagcaagatgtttcttgccttttgttaatgcattgttgtaaagtatttgatgtacattacagattaaagaagaaaagcgcgttgtgtatattacaccaatgccgccgtgtttcctcatctatggttctaaatattgcttcaatttcaaacttttgaaagatgtatggatttccagtttttctttactttctcccagtatgttttaacaaaaaaaaaaaaaagcaggaaaaaaggaatatttagcagtattgttcgttctgatatgtgaatttgtttgtgacaactaaacaaggcattcagcagtttctgacaattaacatacatcattccacactccttgtcaacaaagtgctttttcactgcctaaaattttagatgtagatatttgaaatagattttttcatttataccagttttctttatgatgatacagtgttaaaagaaaataaattacaattgatctgtcatccatatttgctaagaatgtctatttccaagaacctatatcttacaaaatacacattcttgcttgtgtgtctgtgtgtgtgggtgtatgtatgtccacgtactagaaaaattacctgtgtgagattttattttgacagaagctatttcatttgctgttcatcttgtacaaactttgtttttggttttttagtataaatgtacatcagtttgttccttttgtactgtatctattcttctgaccatctagtgactcaggatattaggccagttgaagtggagttataggattttcacacattcacgcatacttggatatcaaccctctctacaggtccgtttccttacacattttatcccctcagaacacgataaaccatggccaattcagtttcactttggggccaattacttgaagtatccacagaaaatgtaaactgctcgccttattcccccaaactttttgttcatgtggcagggagcagtacacaactatggaatagattattaaaactgaacttgaagctaacgttagaatagtggttaacagagctaagaaataaggctaaccatcgaaaagaacatgggccgcgggtaaactagctttgctctttagatgcaagtggctgaggccaataggtgcctagtaaatgttaactgttcttggaaaaaataaaaacatcagtaacaaaagaaa

SEQ ID NO: 27 - protein, 792 aa, Homo sapiens
MRSLSQSQGDPLPPAHTGTYRTSTAPSSPGVDSVPLQRTGSQHGPQNAAAATFQRASYAAGPASNYADPYRQLQYCPSVESPYSKSGPALPPEGTLARSPSIDSIQKDPREFGWRDPELPEVIQMLQHQFPSVQSNAAAYLQHLCFGDNKIKAEIRRQGGIQLLVDLLDHRMTEVHRSACGALRNLVYGKANDDNKIALKNCGGIPALVRLLRKTTDLEIRELVTGVLWNLSSCDALKMPIIQDALAVLTNAVIIPHSGWENSPLQDDRKIQLHSSQVLRNATGCLRNVSSAGEEARRRMRECDGLTDALLYVIQSALGSSEIDSKTVENCVCILRNLSYRLAAETSQGQHMGTDELDGLLCGEANGKDAESSGCWGKKKKKKKSQDQWDGVGPLPDCAEPPKGIQMLWHPSIVKPYLTLLSECSNPDTLEGAAGALQNLAAGSWKWSVYIRAAVRKEKGLPILVELLRIDNDRVVCAVATALRNMALDVRNKELIGKYAMRDLVHRLPGGNNSNNTASKAMSDDTVTAVCCTLHEVITKNMENAKALRDAGGIEKLVGISKSKGDKHSPKVVKAASQVLNSMWQYRDLRSLYKKDGWSQYHFVASSSTIERDRQRPYSSSRTPSISPVRVSPNNRSASAPASPREMISLKERKTDYECTGSNATYHGAKGEHTSRKDAMTAQNTGISTLYRNSYGAPAEDIKHNQVSAQPVPQEPSRKDYETYQPFQNSTRNYDESFFEDQVHHRPPASEYTMHLGLKSTGNYVDFYSAARPYSELNYETSHYPASPDSWV

SEQ ID NO: 28 - DNA, 4790 bp, Homo sapiens
actgggcttgtggtatctctgatatgtcttgctttgctttcttttaaactgtttgcgagtgaaagcagaaattaccataggataattcagcgtcctgactttttcgaggctgttcttcactcccttctcggtttttgagctgctctgtggttccttttagccgtggtgcagggagggaggagggaggtgggtaggatgccaggaaccgcctgctccacagcctggggaaggagggaagagtaacccttcatctttgggactagtcgtcttgtttgcctagactacagttttcaagtatttaaggaccatttatttcaaaaggtacagtaaaggaattacagtttgaaaggctgacccgagagctggaggctgaacggcagatcgtagccagccagctggagcgatgcaagctcggatccgagactggcagcatgagcagcatgagttcagcagaagagcagtttcagtggcagtcacaagatggtcaaaaagatatcgaagatgagcttacaacaggtctcgagctggtggactcctgtattaggtcactacaggaatcaggaatacttgacccacaggattattctacaggtgaaactggttcccgagcctcatacagcagccagcatgggcacctgggcccagagttgcgggccctgcagtccccagaacaccacatagatcccatctatgaagaccgcgtctatcagaagccccctatgaggagtctcagccagagccagggggaccctctgccgccagcacacaccggcacctaccgcacgagcacagccccatcttcccctggtgtcgactccgtccccttgcagcgcacaggcagccagcacggcccacagaatgccgccgcggccaccttccagagggccagctatgccgccggcccagcctccaattacgcggacccctaccgacagctgcagtattgtccctctgttgagtctccatacagcaaatccggccctgctctcccgcctgaaggcaccttggccaggtccccgtccattgatagcattcagaaagatcccagagaatttggatggagagacccggaactgccggaagtgattcagatgttgcagcaccagtttccctcggtccagtctaacgcggcagcctacttgcaacacctctgttttggagacaacaaaattaaagccgagataaggagacaaggaggcatccagctcctggtggacctgttggatcatcggatgaccgaagtccaccgtagtgcctgtggagctctgagaaacctggtgtatgggaaggccaacgatgataacaaaattgccctgaaaaactgtggtggcatcccagcactggtgaggttactccgcaagacgactgacctggagatccgggagctggtcacaggagtcctttggaacctctcctcatgcgatgcactcaaaatgccaatcatccaggatgccctagcagtactgaccaacgcggtgattatcccccactcaggctgggaaaattcgcctcttcaggatgatcggaaaatacagctgcattcatcacaggtgctgcgtaacgccaccgggtgcctaaggaatgttagttcggccggagaggaggcccgcagaaggatgagagagtgtgatgggcttacggatgccttgctgtacgtgatccagtctgcgctggggagcagtgagatcgatagcaagaccgttgaaaactgtgtgtgcattttaaggaacctctcgtaccggctggcggcagaaacgtctcagggacagcacatgggcacggacgagctggacgggctactctgtggcgaggccaatggcaaggatgctgagagctctgggtgctggggcaagaagaagaagaaaaagaaatcccaagatcagtgggatggagtaggacctcttccagactgtgctgaaccaccaaaagggatccagatgctgtggcacccatcaatagtcaaaccctacctcacactgctctctgagtgctcaaatccagacacgctggaaggggcggcaggcgccctgcagaacttggctgcagggagctggaagggctgggctgaggatgtggcaggcatggcatatgccctacgttcactgccagagggggctccctgcctgccacagtggtcagtatatatccgagccgctgtccgaaaagagaaaggcctgcccatcctcgtggagctgctccgaatagacaatgaccgtgtggtgtgcgcggtggccactgcgctgcggaacatggccttggacgtcagaaataaggagctcatcggcaaatacgccatgcgagacctagtccacaggcttccaggagggaacaacagcaacaacactgcaagcaaggccatgtcggatgacacagtgacagctgtctgctgcacactgcacgaagtgattaccaagaacatggagaacgccaaggccttacgggatgccggtggcatcgagaagttggtcggcatctccaaaagcaaaggagataaacactctccaaaagtggtcaaggctgcatctcaggtcctcaacagcatgtggcagtaccgagatctgaggagtctctacaaaaaggatggatggtcacaataccactttgtagcctcgtcttcaaccatcgagagggaccggcaaaggccctactcctcctcccgcacgccctccatctcccctgtgcgcgtgtctcccaacaaccgctcagcaagtgccccagcttcacctcgggaaatgatcagcctcaaagaaaggaaaacagactacgagtgcaccggcagcaacgccacctaccacggagctaaaggcgaacacacttccaggaaagatgccatgacagctcaaaacactggaatttcaactttgtataggaattcttatggtgcgcccgctgaagacatcaaacacaaccaggtttcagcacagccagtcccacaggagcccagcagaaaagattacgagacctaccagccatttcagaattccacaagaaattacgatgagtccttcttcgaggaccaggtccaccatcgccctcccgccagcgagtacaccatgcacctgggtctcaagtccaccggcaactacgttgacttctactcagctgcccgtccctacagtgaactgaactatgaaacgagccactacccggcctcccccgactcctgggtgtgaggagcagggcacaggcgctccgggaacagtgcatgtgcatgcataccacaagacatttctttctgttttgtttttttctcctgcaaatttagtttgttaaagcctgttccataggaaggctgtgataaccagtaaggaaatattaagagctattttagaaagctaaatgaatcgcaagttaacttggaaatcagtagaaagctaaagtgatcctaaatatgacagtgggcagcacctttctagcgtgagctgtagagtaacgagaagtgctttatactgaacgtggttgatgggaggagagacgaggcattcgggccggtggggcgtaagggttatcgttaagcacaagacacagaatagtttacacactgtgtgggggacggcttctcacgctttgtttactctcttcatccgttgtgactctaggcttcaggttgcattggggttcctctgtacagcaagatgtttcttgccttttgttaatgcattgttgtaaagtatttgatgtacattacagattaaagaagaaaagcgcgttgtgtatattacaccaatgccgccgtgtttcctcatctatggttctaaatattgcttcaatttcaaacttttgaaagatgtatggatttccagtttttctttactttctcccagtatgttttaacaaaaaaaaaaaaaagcaggaaaaaaggaatatttagcagtattgttcgttctgatatgtgaatttgtttgtgacaactaaacaaggcattcagcagtttctgacaattaacatacatcattccacactccttgtcaacaaagtgctttttcactgcctaaaattttagatgtagatatttgaaatagattttttcatttataccagttttctttatgatgatacagtgttaaaagaaaataaattacaattgatctgtcatccatatttgctaagaatgtctatttccaagaacctatatcttacaaaatacacattcttgcttgtgtgtctgtgtgtgtgggtgtatgtatgtccacgtactagaaaaattacctgtgtgagattttattttgacagaagctatttcatttgctgttcatcttgtacaaactttgtttttggttttttagtataaatgtacatcagtttgttccttttgtactgtatctattcttctgaccatctagtgactcaggatattaggccagttgaagtggagttataggattttcacacattcacgcatacttggatatcaaccctctctacaggtccgtttccttacacattttatcccctcagaacacgataaaccatggccaattcagtttcactttggggccaattacttgaagtatccacagaaaatgtaaactgctcgccttattcccccaaactttttgttcatgtggcagggagcagtacacaactatggaatagattattaaaactgaacttgaagctaacgttagaatagtggttaacagagctaagaaataaggctaaccatcgaaaagaacatgggccgcgggtaaactagctttgctctttagatgcaagtggctgaggccaataggtgcctagtaaatgttaactgttcttggaaaaaataaaaacatcagtaacaaaagaaa

SEQ ID NO: 29 - protein, 913 aa, Homo sapiens
MSSMSSAEEQFQWQSQDGQKDIEDELTTGLELVDSCIRSLQESGILDPQDYSTGETGSRASYSSQHGHLGPELRALQSPEHHIDPIYEDRVYQKPPMRSLSQSQGDPLPPAHTGTYRTSTAPSSPGVDSVPLQRTGSQHGPQNAAAATFQRASYAAGPASNYADPYRQLQYCPSVESPYSKSGPALPPEGTLARSPSIDSIQKDPREFGWRDPELPEVIQMLQHQFPSVQSNAAAYLQHLCFGDNKIKAEIRRQGGIQLLVDLLDHRMTEVHRSACGALRNLVYGKANDDNKIALKNCGGIPALVRLLRKTTDLEIRELVTGVLWNLSSCDALKMPIIQDALAVLTNAVIIPHSGWENSPLQDDRKIQLHSSQVLRNATGCLRNVSSAGEEARRRMRECDGLTDALLYVIQSALGSSEIDSKTVENCVCILRNLSYRLAAETSQGQHMGTDELDGLLCGEANGKDAESSGCWGKKKKKKKSQDQWDGVGPLPDCAEPPKGIQMLWHPSIVKPYLTLLSECSNPDTLEGAAGALQNLAAGSWKGWAEDVAGMAYALRSLPEGAPCLPQWSVYIRAAVRKEKGLPILVELLRIDNDRVVCAVATALRNMALDVRNKELIGKYAMRDLVHRLPGGNNSNNTASKAMSDDTVTAVCCTLHEVITKNMENAKALRDAGGIEKLVGISKSKGDKHSPKVVKAASQVLNSMWQYRDLRSLYKKDGWSQYHFVASSSTIERDRQRPYSSSRTPSISPVRVSPNNRSASAPASPREMISLKERKTDYECTGSNATYHGAKGEHTSRKDAMTAQNTGISTLYRNSYGAPAEDIKHNQVSAQPVPQEPSRKDYETYQPFQNSTRNYDESFFEDQVHHRPPASEYTMHLGLKSTGNYVDFYSAARPYSELNYETSHYPASPDSWV

SEQ ID NO: 30 - DNA, 5305 bp, Homo sapiens
atgtttgcgaggaagccgccgggcgccgcgcctttgggagctatgcctgttccagaccagccttcatcagcctcagagaagacgagttccctgagccccggcttaaacacctccaacggggatggctctgaaacagaaaccacctctgccatcctcgcctcagtcaaagaacaggaattacagtttgaaaggctgacccgagagctggaggctgaacggcagatcgtagccagccagctggagcgatgcaagctcggatccgagactggcagcatgagcagcatgagttcagcagaagagcagtttcagtggcagtcacaagatggtcaaaaagatatcgaagatgagcttacaacaggtctcgagctggtggactcctgtattaggtcactacaggaatcaggaatacttgacccacaggattattctacaggtgaaaggcccagcctgctctcccagagtgcacttcagctcaattccaaacctgaagggtctttccagtatccggccagctaccatagcaaccagaccctggccctgggggaaaccaccccttcacagctcccggcccgaggcacacaagcccgagctacgggccagagcttcagccagggcacgaccagccgcgccggccacctggcggggcccgagcccgcgccgccgccgccgccgccgccgcgggagccgttcgcgcccagcctgggcagcgccttccacctgcccgacgcgccgcccgccgccgccgccgccgcgctctactactccagctccacgctgcccgcgccgccgcgcgggggctccccgctggccgcgccccagggcggttcgcccaccaagctgcagcgcggcggctcggcccccgagggcgccacctacgccgcgccgcgcggctcctcgcccaagcagtcgcccagccgcctggccaagtcctacagcaccagctcgcccatcaacatcgtcgtgtcctcggccggcctgtccccgatccgcgtgacctcgccccccaccgtgcagtccaccatctcctcctcgcccatccaccagctgagctccaccatcggcacgtacgccaccctgtcgcccaccaagcgcctggtccacgcgtccgagcagtacagcaagcactcgcaggagctgtatgccacggccaccctccagaggccgggcagcctggcagctggttcccgagcctcatacagcagccagcatgggcacctgggcccagagttgcgggccctgcagtccccagaacaccacatagatcccatctatgaagtccgcgtctatcagaagccccctatgaggagtctcagccagagccagggggtccctctgccgccagcacacaccggcacctaccgcacgagcacagccccatcttcccctggtgtcgactccgtccccttgcagcgcacaggcagccagcacggcccacagaatgccgccgcggccaccttccagagggccagctatgccgccggcccagcctccaattacgcggacccctaccgacagctgcagtattgtccctctgttgagtctccatacagcaaatccggccctgctctcccgcctgaaggcaccttggccaggtccccgtccattgatagcattcagaaagatcccagagaatttggatggagagacccggaactgccggaagtgattcagatgttgcagcaccagtttccctcggtccagtctaacgcggcagcctacttgcaacacctctgttttggagacaacaaaattaaagccgagataaggagacaaggaggcatccagctcctggtggacctgttggatcatcggatgaccgaagtccaccgtagtgcctgtggagctctgagaaacctggtgtatgggaaggccaacgatgataacaaaattgccctgaaaaactgtggtggcatcccagcactggtgaggttactccgcaagacgactgacctggagatccgggagctggtcacaggagtcctttggaacctctcctcatgcgatgcactcaaaatgccaatcatccaggatgccctagcagtactgaccaacgcggtgattatcccccactcaggctgggaaaattcgcctcttcaggatgatcggaaaatacagctgcattcatcacaggtgctgcgtaacgccaccgggtgcttaaggaatgttagttcgcccggagaggaggcccgcagaaggatgagagagtgtgatgggcttacggatgccttgctgtacgtgatccagtctgcgctggggagcagtgagatcgatagcaagaccgttgaaaactgtgtgtgcattttaaggaacctctcgtaccggctggcggcagaaacgtctcagggacagcacatgggcacggacgagctggacgggctactctgtggcgaggccaatggcaaggatgctgagagctctgggtgctggggcaagaagaagaagaaaaagaaatcccaagatcagtgggatggagtaggacctcttccagactgtgctgaaccaccaaaagggatccagatgctgtggcacccatcaatagtcaaaccctacctcacactgctctctgagtgctcaaatccagacacgctggaaggggcggcaggcgccctgcagaacttggctgcagggagctggaagtggtcagtatatatccgagccgctgtccgaaaagagaaaggccggcccatcctcgtggagctgctccgaatagacaatgaccgtgtggcgtgcgcggtggccactgcgctgcggaacatggccttggacgtcagaaataaggagctcatcggcaaatacgccatgcgagacctagtccacaggcttccaggagggaacaacagcaacaacactgcaagcaaggccatgtcggatgacacagtgacagctgtctgctgcacactgcacgaagtgattaccaagaacatggagaacgccaaggccttacgggatgccggtggcatcgagaagttggtcggcatctccaaaagcaaaggagataaacactctccaaaagtggtcaaggctgcatctcaggtcctcaacagcatgtggcagtaccgagatctgaggagtctctacaaaaaggatggatggtcacaataccactttgtagcctcgtcttcaaccatcgagagggaccggcaaaggccctactcctcctcccgcacgccctccatctcccctgtgcgcgtgtctcccaacaaccgctcagcaagtgccccagcttcacctcgggaaatgatcagcctcaaagaaaggaaaacagactacgagtgcaccggcagcaacgccacctaccacggagctaaaggcgaacacacttccaggaaagatgccatgacagctcaaaacactggaatttcaactttgtataggaattcttatggtgcgcccgctgaagacatcaaacacaaccaggtttcagcacagccagtcccacaggagcccagcagaaaagattacgagacctaccagccatttcagaattccacaagaaattacgatgagtccttcttcgaggaccaggtccaccatcgccctcccgccagcgagtacaccatgcacctgggtctcaagtccaccggcaactacgttgacttctactcagctgcccgtccctacagtgaactgaactatgaaacgagccactacccggcctcccccgactcctgggtgtgaggagcagggcacaggcgctccgggaacagtgcatgtgcatgcataccacaagacatttctttctgttttgtttttttctcctgcaaatttagtttgttaaagcctgttccataggaaggctgtgataaccagtaaggaaatattaagagctattttagaaagctaaatgaatcgcaagtttaacttggaaatcagtagaaagctaaagtgatcctaaatatgacagtgggcagcacctttctagcgtgagctgtaaagtaacgagaagtgctttatactgaacgtggttgatgggaggagagacgaggcattcgggccggtggggcgtaagggttatcgttaagcacaagacacagaatagtttacacactgtgtgggggacggcttctcacgctttgtttactctcttcatccgttgtgactctaggcttcaggttgcattggggttcctctgtacagcaagatgtttcttgccttttgttaatgcattgttgtaaagtatttgatgtacattacagattaaagaagaaaagcgcgttgtgtatattacaccaatgccgccgtgtttcctcatctatggttctaaatattgcttcaatttcaaacttttgaaagatgtatggatttccagtttttctttactttctcccagtatgttttaacaaaaaaaaaaaaaagcaggaaaaaaggaatatttagcagtattgttcgttctgatatgtgaatttgtttgtgacaactaaacaaggcattcagcagtttctgacaattaacatacatcattccacactccttgtcaacaaagtgctttttcactgcctaaaattttagatgtagatatttgaaatagattttttcatttataccagttttctttatgatgatacagtgttaaaagaaaataaattacaattgatctgtcatccatatttgctaagaatgtctatttccaagaacctatatcttacaaaatacacattcttgcttgtgtgtctgtgtgtgtgggtgtatgtatgtccacgtactagaaaaattacctgtgtgagattttattttgacagaagctatttcatttgctgttcatcttgtacaaactttgtttttggttttttagtataaatgtacatcagtttgttccttttgtactgtatctattcttctgaccatctagtgactcaggatattaggcccagttgaagtggagttatggggattttccacacattcacgcatacttggatatcaaccctctctacaggtccgtttccttacacattttatccccctcagaacacgataaaccatggccaattcagtttcactttggggcccaattacttgaagtatcccacagaaaatgtaaactgctcgccttattcccccaaactttttgttcatgtggcagggagcagtacacaactatggaatagattattaaaactgaacttgaagctaacgttagaatagtggttaacagagctaagaaataaggctaaccatcgaaaagaacatgggccgcgggtaaactagctttgctctttagatgcaagtggctgaggccaataggtgcctagtaaatgttaactgttcttggaaaaaataaaaacatcagtaacaaaaaaaaaaa

SEQ ID NO: 31 - protein, 1225 aa, Homo sapiens; identical to SEQ ID NO: 21

SEQ ID NO: 32 - DNA, 3678 bp, Homo sapiens
atgtttgcgaggaagccgccgggcgccgcgcctttgggagctatgcctgttccagaccagccttcatcagcctcagagaagacgagttccctgagccccggcttaaacacctccaacggggatggctctgaaacagaaaccacctctgccatcctcgcctcagtcaaagaacaggaattacagtttgaaaggctgacccgagagctggaggctgaacggcagatcgtagccagccagctggagcgatgcaagctcggatccgagactggcagcatgagcagcatgagttcagcagaagagcagtttcagtggcagtcacaagatggtcaaaaagatatcgaagatgagcttacaacaggtctcgagctggtggactcctgtattaggtcactacaggaatcaggaatacttgacccacaggattattctacaggtgaaaggcccagcctgctctcccagagtgcacttcagctcaattccaaacctgaagggtctttccagtatccggccagctaccatagcaaccagaccctggccctgggggaaaccaccccttcacagctcccggcccgaggcacacaagcccgagctacgggccagagcttcagccagggcacgaccagccgcgccggccacctggcggggcccgagcccgcgccgccgccgccgccgccgccgcgggagccgttcgcgcccagcctgggcagcgccttccacctgcccgacgcgccgcccgccgccgccgccgccgcgctctactactccagctccacgctgcccgcgccgccgcgcgggggctccccgctggccgcgccccagggcggtgcacccaccaagctgcagcgcggcggctcggcccccgagggcgccacctacgccgcgccgcgcggctcctcgcccaagcagtcgcccagccgcctggccaagtcctacagcaccagctcgcccatcaacatcgtcgtgtcctcggccggcctgtccccgatccgcgtgacctcgccccccaccgtgcagtccaccatctcctcctcgcccatccaccagctgagctccaccatcggcacgtacgccaccctgtcgcccaccaagcgcctggtccacgcgtccgagcagtacagcaagcactcgcaggagctgtatgccacggccaccctccagaggccgggcagcctggcagctggttcccgagcctcatacagcagccagcatgggcacctgggcccagagttgcgggccctgcagtccccagaacaccacatagatcccatctatgaagaccgcgtctatcagaagccccctatgaggagtctcagccagagccagggggaccctctgccgccagcacacaccggcacctaccgcacgagcacagccccatcttcccctggtgtcgactccgtccccttgcagcgcacaggcagccagcacggcccacagaatgccgccgcggccaccttccagagggccagctatgccgccggcccagcctccaattacgcggacccctaccgacagctgcagtattgtccctctgttgagtctccatacagcaaatccggccctgctctcccgcctgaaggcaccttggccaggtccccgtccattgatagcattcagaaagatcccagagaatttggatggagagacccggaactgccggaagtgattcagatgttgcagcaccagtttccctcggtccagtctaacgcggcagcctacttgcaacacctctgttttggagacaacaaaattaaagccgagataaggagacaaggaggcatccagctcctggtggacctgttggatcatcggatgaccgaagtccaccgtagtgcctgtggagctctgagaaacctggtgtatgggaaggccaacgatgataacaaaattgccctgaaaaactgtggtggcatcccagcactggtgaggttactccgcaagacgactgacctggagatccgggagctggtcacaggagtcctttggaacctctcctcatgcgatgcactcaaaatgccaatcatccaggatgccctagcagtactgaccaacgcggtgattatcccccactcaggctgggaaaattcgcctcttcaggatgatcggaaaatacagctgcattcatcacaggtgctgcgtaacgccaccgggtgcctaaggaatgttagttcggccggagaggaggcccgcagaaggatgagagagtgtgatgggcttacggatgccttgctgtacgtgatccagtctgcgctggggagcagtgagatcgatagcaagaccgttgaaaactgtgtgtgcattttaaggaacctctcgtaccggctggcggcagaaacgtctcagggacagcacatgggcacggacgagctggacgggctactctgtggcgaggccaatggcaaggatgctgagagctctgggtgctggggcaagaagaagaagaaaaagaaatcccaagatcagtgggatggagtaggacctcttccagactgtgctgaaccaccaaaagggatccagatgctgtggcacccatcaatagtcaaaccctacctcacactgctctctgagtgctcaaatccagacacgctggaaggggcggcaggcgccctgcagaacttggctgcagggagctggaagtggtcagtatatatccgagccgctgtccgaaaagagaaaggcctgcccatcctcgtggagctgctccgaatagacaatgaccgtgtggtgtgcgcggtggccactgcgctgcggaacatggccttggacgtcagaaataaggagctcatcggcaaatacgccatgcgagacctagtccacaggcttccaggagggaacaacagcaacaacactgcaagcaaggccatgtcggatgacacagtgacagctgtctgctgcacactgcacgaagtgattaccaagaacatggagaacgccaaggccttacgggatgccggtggcatcgagaagttggtcggcatctccaaaagcaaaggagataaacactctccaaaagtggtcaaggctgcatctcaggtcctcaacagcatgtggcagtaccgagatctgaggagtctctacaaaaaggatggatggtcacaataccactttgtagcctcgtcttcaaccatcgagagggaccggcaaaggccctactcctcctcccgcacgccctccatctcccctgtgcgcgtgtctcccaacaaccgctcagcaagtgccccagcttcacctcgggaaatgatcagcctcaaagaaaggaaaacagactacgagtgcaccggcagcaacgccacctaccacggagctaaaggcgaacacacttccaggaaagatgccatgacagctcaaaacactggaatttcaactttgtataggaattcttatggtgcgcccgctgaagacatcaaacacaaccaggtttcagcacagccagtcccacaggagcccagcagaaaagattacgagacctaccagccatttcagaattccacaagaaattacgatgagtccttcttcgaggaccaggtccaccatcgccctcccgccagcgagtacaccatgcacctgggtctcaagtccaccggcaactacgttgacttctactcagctgcccgtccctacagtgaactgaactatgaaacgagccactacccggcctcccccgactcctgggtgtga

SEQ ID NO: 33 - protein, 1225 aa, Homo sapiens
MFARKPPGAAPLGAMPVPDQPSSASEKTSSLSPGLNTSNGDGSETETTSAILASVKEQELQFERLTRELEAERQIVASQLERCKLGSETGSMSSMSSAEEQFQWQSQDGQKDIEDELTTGLELVDSCIRSLQESGILDPQDYSTGERPSLLSQSALQLNSKPEGSFQYPASYHSNQTLALGETTPSQLPARGTQARATGQSFSQGTTSRAGHLAGPEPAPPPPPPPREPFAPSLGSAFHLPDAPPAAAAAALYYSSSTLPAPPRGGSPLAAPQGGAPTKLQRGGSAPEGATYAAPRGSSPKQSPSRLAKSYSTSSPINIVVSSAGLSPIRVTSPPTVQSTISSSPIHQLSSTIGTYATLSPTKRLVHASEQYSKHSQELYATATLQRPGSLAAGSRASYSSQHGHLGPELRALQSPEHHIDPIYEDRVYQKPPMRSLSQSQGDPLPPAHTGTYRTSTAPSSPGVDSVPLQRTGSQHGPQNAAAATFQRASYAAGPASNYADPYRQLQYCPSVESPYSKSGPALPPEGTLARSPSIDSIQKDPREFGWRDPELPEVIQMLQHQFPSVQSNAAAYLQHLCFGDNKIKAEIRRQGGIQLLVDLLDHRMTEVHRSACGALRNLVYGKANDDNKIALKNCGGIPALVRLLRKTTDLEIRELVTGVLWNLSSCDALKMPIIQDALAVLTNAVIIPHSGWENSPLQDDRKIQLHSSQVLRNATGCLRNVSSAGEEARRRMRECDGLTDALLYVIQSALGSSEIDSKTVENCVCILRNLSYRLAAETSQGQHMGTDELDGLLCGEANGKDAESSGCWGKKKKKKKSQDQWDGVGPLPDCAEPPKGIQMLWHPSIVKPYLTLLSECSNPDTLEGAAGALQNLAAGSWKWSVYIRAAVRKEKGLPILVELLRIDNDRVVCAVATALRNMALDVRNKELIGKYAMRDLVHRLPGGNNSNNTASKAMSDDTVTAVCCTLHEVITKNMENAKALRDAGGIEKLVGISKSKGDKHSPKVVKAASQVLNSMWQYRDLRSLYKKDGWSQYHFVASSSTIERDRQRPYSSSRTPSISPVRVSPNNRSASAPASPREMISLKERKTDYECTGSNATYHGAKGEHTSRKDAMTAQNTGISTLYRNSYGAPAEDIKHNQVSAQPVPQEPSRKDYETYQPFQNSTRNYDESFFEDQVHHRPPASEYTMHLGLKSTGNYVDFYSAARPYSELNYETSHYPASPDSWV